SEQUENCE LISTING (1 sequence)

SEQ ID NO: 1 - DNA, 5580 bp, synthetic construct
  misc_feature 1..5580: note = clDNA
  source 1..5580: mol_type = other DNA
gcgtataatggactattgtgtgctgataggtctcgccctgatttaaatcgtcgaccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcccgggatccaccggtcgccaccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccatttaaatcggtctcgagggtatcagcacacaattgcccattatacgcgcgtataatgggcaattgtgtgctgataccctcgagaccgatttaaatggtcgactaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccgcggccgctttacaatttggactttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgccacgcccgcgtcgaagatgttggggtgttgtaacaatatcgattccaattcagcgggggccacctgatatcctttgtatttaattaaagacttcaagcggtcaactatgaagaagtgttcgtcttcgtcccagtaagctatgtctccagaatgtagccatccatccttgtcaatcaaggcgttggtcgcttccggattgtttacataaccggacataatcataggtcctctgacacataattcgcctctctgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgtcgtatccctggaagatggaagcgttttgcaaccgcttccccgacttctttcgaaagaggtgcgcccccagaagcaatttcgtgtaaattagataaatcgtatttgtcaatcagagtgcttttggcgaagaatgaaaatagggttggtactagcaacgcactttgaattttgtaatcctgaagggatcgtaaaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctgacgcaggcagttctatgcggaagggccacacccttaggtaacccagtagatccagaggaattcattatcagtgcaattgttttgtcacgatcaaaggactctggtacaaaatcgtattcattaaaaccgggaggtagatgagatgtgacgaacgtgtacatcgactgaaatccctggtaatccgttttagaatccatgataataattttctggattattggtaattttttttgcacgttcaaaattttttgcaacccctttttggaaacaaacactacggtaggctgcgaaatgttcatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtattccgcgtacgtgatgttcacctcgatatgtgcatctgtaaaagcaattgttccaggaaccagggcgtatctcttcatagccttatgcagttgctctccagcggttccatcctctagaggatagaatggcgccgggcctttctttatgtttttggcgtcttccatggtggcgaccggtggatcccgggccgcgggtacaattccgcagcttttagagcagaagtaacacttccgtacaggcctagaagtaaaggcaacatccactgaggagcagttctttgatttgcaccaccaccggatccgggacctgaaataaaagacaaaaagactaaacttaccagttaactttctggtttttcagttcctcgagtaccggatcctctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacggtcgacgatttaaatcagggcgagacctatcagcacacaatagtccattatacgc

(19) United States
(12) Patent Application Publication
(10) Pub. No.: US 2025/0101175 A1
(43) Pub. Date: Mar. 27, 2025

(54) STAR-SHAPED PASP-OLIGOAMINE DERIVATIVES

(71) Applicant: POLYPEPTIDE THERAPEUTIC SOLUTIONS, S.L., PATERNA (ES)

(72) Inventors: Lidia HERRERA MUÑOZ, PATERNA (ES); Irene DOLZ PÉREZ, PATERNA (ES); Carles FELIP LEON, PATERNA (ES); Vicent Josep NEBOT CARDA, PATERNA (ES); Maria Jesús VICENT DOCÓN, VALENCIA (ES)

(73) Assignee: POLYPEPTIDE THERAPEUTIC SOLUTIONS, S.L., PATERNA (ES)

(21) Appl. No.: 18/291,124

(22) PCT Filed: Jul. 22, 2022
(86) PCT No.: PCT/EP2022/070604

§ 371 Date: Jan. 22, 2024

(30) Foreign Application Priority Data
Jul. 22, 2021 (EP) 21382666.2

(51) Int. Cl. C08G 69/36; A61K 47/64; C12N 15/88

(52) U.S. Cl. CPC C08G 69/36; A61K 47/6455; C12N 15/88

(57) ABSTRACT

The present disclosure relates to 3-arm star-shaped polycationically charged polymers consisting of a 1,3,5-benzenetricarboxamide related central core and 3 polypeptide backbone arms and its use as carriers for the delivery of active agents, such as nucleic acids.

This application claims the benefit of European Patent Application EP21382666.2 filed on Jul. 22, 2021.

### TECHNICAL FIELD

The present disclosure relates to 3-arm star-shaped polypeptide derivatives which are able to deliver active agents and/or imaging agents to target cells or tissues, and relates more specifically, to 3-arm star-shaped polycationically charged polymers and its use as carriers for the delivery of active agents, such as nucleic acids, and/or imaging agents.

### BACKGROUND ART

There has been a considerable effort to the development of new polymeric structures with specific properties to be used as targeted drugs, including large molecules such as polypeptides and nucleic acids, delivery systems.

There have been described some polycationically charged polymers which spontaneously form spherical micelles or nanoobjects with an anionic macromolecule, due to the electrostatic interaction acting between the two in aqueous/buffered media.

EP3331937 discloses a family of 3-arm star shaped polypeptide derivatives consisting of a 1,3,5-benzenetricarboxamide related central core employed as the initiator for the ring opening polymerization of N-carboxyanhydride monomers and 3 polypeptide backbone arms. The polypeptidic backbone of the compounds described therein is different from those herein disclosed. According to EP3331937, the 3-arm star-shaped polypeptide derivatives undergo a self-assembly process yielding bigger nanometric globular structures, with hard sphere shapes bearing branching points outside directed. In the 3-arm star-shaped polypeptide derivatives according to formula (II) therein described, only between 0.01% to 50% of the glutamic acid units of St-PGAs are modified.

It is well known that gene therapy requires an appropriate technology for delivery thereof to target cells because of low in vivo stability of a nucleic acid molecule. For example, as the delivery technology for a nucleic acid, hitherto, there has been known a technology utilizing a block copolymer having a hydrophilic polymer segment and a cationic polymer segment to form a complex with a nucleic acid via an electrostatic interaction (polyion complex).

Examples of known cationic polyamino acids include linear poly(N—[N-(2-aminoethyl]aspartamide) (PAsp(DET)), which has a ethylenediamine structure in a side chain thereof and a block copolymer containing the PAsp(DET) as one block component thereof. It is known that PAsp(DET) form polyplexes with nucleic acids and may facilitate introducing the plasmid DNA into cells with high efficiency, to thereby express a gene encoded in the nucleic acid. Other linear PAsp(DET) derivatives are also known to form polyplexes.

From what it is known in the field, there is still a need to find new carriers for specific and controlled delivering of active agents and/or imaging agents to target cell or tissues.

### SUMMARY

The present disclosure has been made in order to solve the problem of the related techniques, and a primary object of the present disclosure is to provide new carriers for biomedical applications. In particular, 3-arm star-shaped polypeptide derivatives which are able to deliver active agents and/or an imaging agents to target cells or tissues.

The 3-arm star-shaped polypeptide derivatives of the present disclosure are 3-arm star-shaped polycationically charged polymers consisting of a 1,3,5-benzenetricarboxamide related central core and 3 polypeptide backbone arms.

In the context of the present invention, the terms “cationically charged”, “polycationically charged” or equivalents, refer to polymers that have an amino-protonable (i.e. cationic) group in the side-chain, i.e. those which has already been rendered cationic with a coordinated hydrogen ion, but it also comprise an amino group that will be cationic once it gains a hydrogen ion. The polypeptides having a cationic group in the side-chain comprises polypeptides obtained through peptide bond of known amino acids having basic side-chains (e.g. lysine, arginine, histidine, ornithine, proline, etc) as well as polypeptides obtained through peptide bond of any amino acid and subsequent substitution in the side-chain to have a cationically charged group.

The three-dimensional structuring and subsequent supramolecular organization of non-viral vectors has been shown to be a key feature for increased efficiency in the transfection process. Vectors based on liposomes, polymersomes, comb-like structures or dendrimers have been shown to have transfection efficiencies superior to simple cationic polymeric systems. This difference is mainly based on the high density of the surface of the vectors, as well as on the greater ability to condense the DNA, promoting anchorage and increasing its transfection capacity.

Among the vectors that adopt a three-dimensional structure, cationically charged star-shaped polymers have recently been explored and have shown to be highly promising non-viral vectors. Polyplexes based on star systems present well-defined architectures with predictable structures and conformations (generally spherical, facilitating endocytosis and maximizing the transfection process), high homogeneity, high multivalence, multifunctionality and response to stimuli, as well as greater capacity to encapsulation, better solubility and tunable physical properties in terms of rheology, mechanical and thermal properties. In addition, these structures allow more precise control over supramolecular morphologies that can result in improved biodistribution, pharmacokinetics and improved penetration of biological barriers.

A first aspect of the present disclosure relates to a compound of formula I, a pharmaceutically acceptable salt thereof, or any stereoisomer or mixtures of stereoisomers, either of the compound of formula (I) or of any of its pharmaceutically acceptable salts, comprising homo-polypeptides or random or block or graft co-polypeptides:

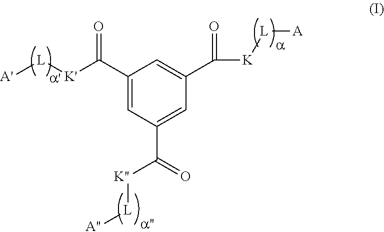

wherein A, A′ and A″ are each independently selected from a radical of formula II; and each of A, A′ and A″ sub-units may be the same or different;

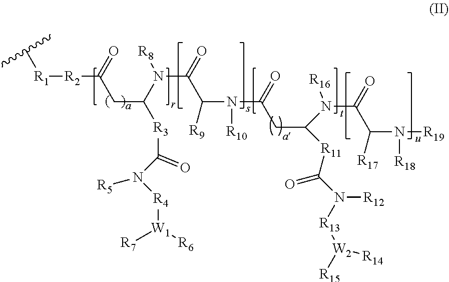

wherein the wavy line denotes the attaching point;
and though the repeating units defined by square brackets with their numerical value, r, s, t and u respectively in the formula II are shown in a particular order for convenience of description, the repeating units may be present in any order and the repeating units may be block or randomly present; and wherein each of the repeating units, may comprise blocks of monomer units which may be the same or different from each other;
wherein K, K′ and K″ are each independently selected from —O— and —NH—;
L is selected from

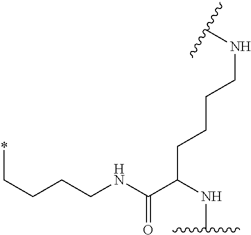 [SMILES: *CCCCNC(=O)C(CCCCN*)N*]

wherein α, α′ and α″ are an integer from 0 to 1; each wavy lines denote the attaching points to A, A′ or A″; and “*” denotes the attaching point to K, K′ or K″;
wherein R2 is selected from —O— and —NH—
wherein R1 is a biradical selected from the group consisting of (III) and (IV)

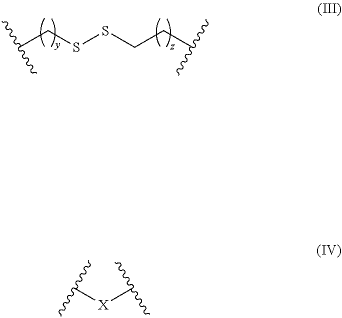

wherein the wavy lines denote the attaching points;
wherein y and z are integers independently ranging from 1 to 20;
X is a biradical selected from the group consisting of —NH—, —NH(C₁-C₆)alkyl-, —O—, —(C₁-C₆)alkyl-COO—, a straight or branched —(C₁-C₃₀)alkylene-, and a biradical selected from the group consisting of (V), (VI), (VII), (VIII), (IX), (X), (XI) and (XII)

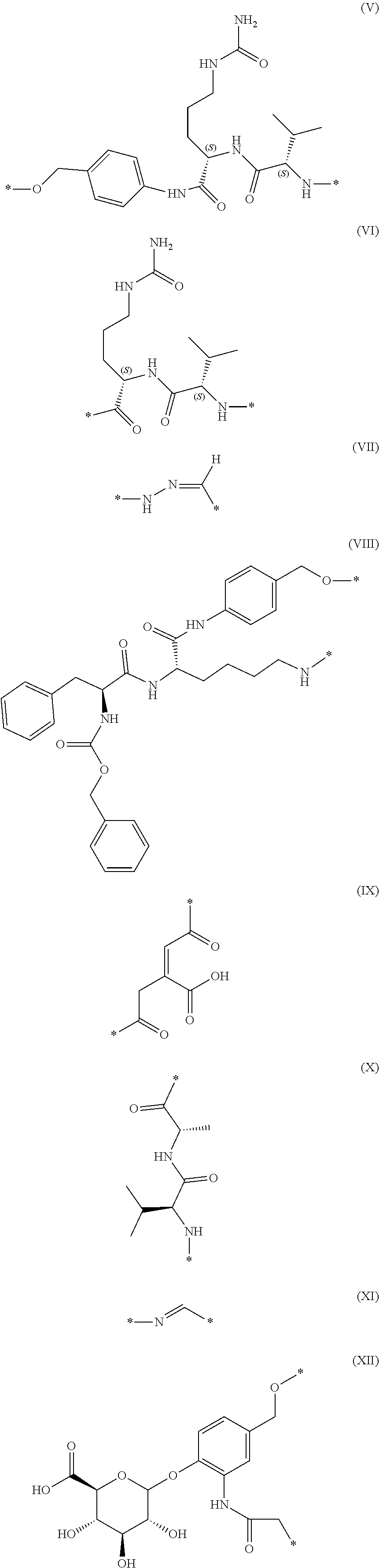

wherein the “*” denotes the attaching points;
wherein the —(C₁-C₃₀)alkylene biradical of X is optionally substituted with one or more radicals selected from the group consisting of —OH, —NRₐR_{b}, —SH, —NHNH₂, —COOR_{c}, —CF₃, —OCF₃, and halogen;
Rₐ, R_{b} and R_{c} are independently selected from the group consisting of H, -phenyl, —(C₁-C₃₀)alkyl, —(C₂-C₃₀)alkenyl, —(C₁-C₃₀)alkylphenyl, and -phenyl(C₁-C₃₀)alkyl;
wherein a and a′ are integers independently ranging from 0 to 1;
r, s, t and u are integers independently ranging from 0 to 500, wherein at least one of r or t are >1;
where in the radical of formula (II), the repetitive unit defined by square brackets with the numerical value r is denoted as PAA1; the repetitive unit defined by square brackets with the numerical value s is denoted as PAA2; the repetitive unit defined by square brackets with the numerical value t is denoted as PAA3; and the repetitive unit defined by square brackets with the numerical value u is denoted as PAA4;
wherein the molar ratio of the PAA1 monomer to the PAA2 is from 100/0 to 60/40;
wherein the molar ratio of the PAA1 monomer to the PAA4 is from 100/0 to 60/40;
wherein the molar ratio of the PAA3 monomer to the PAA4 is from 100/0 to 60/40;
wherein the molar ratio of the PAA3 monomer to the PAA2 is from 100/0 to 60/40;
and wherein the molar ratio of the sum of PAA1+PAA3 monomers to the sum of the PAA2+PAA4 is from 100/0 to 60/40;
R₉ and R₁₇ are a radical independently selected from the group consisting of H, —(C₁-C₃₀)alkyl, —(C₂-C₃₀)alkenyl, —(C₁-C₃₀)alkyl-Rⁱ¹, —(C₁-C₃₀)alkyl-COORⁱⁱ¹, —(C₁-C₃₀)alkyl-O—Rⁱⁱ¹, —(C₁-C₃₀)alkyl-NR^{iv1}R^{v1}, —C(O)—R^{v1}, —(C₁-C₁₂)alkyl-CO—NH₂, and a radical selected from the group consisting of (XIII), (XIV), (XV), (XVI), (XVII), (XVIII), (XIX), (XX), and (XXI)

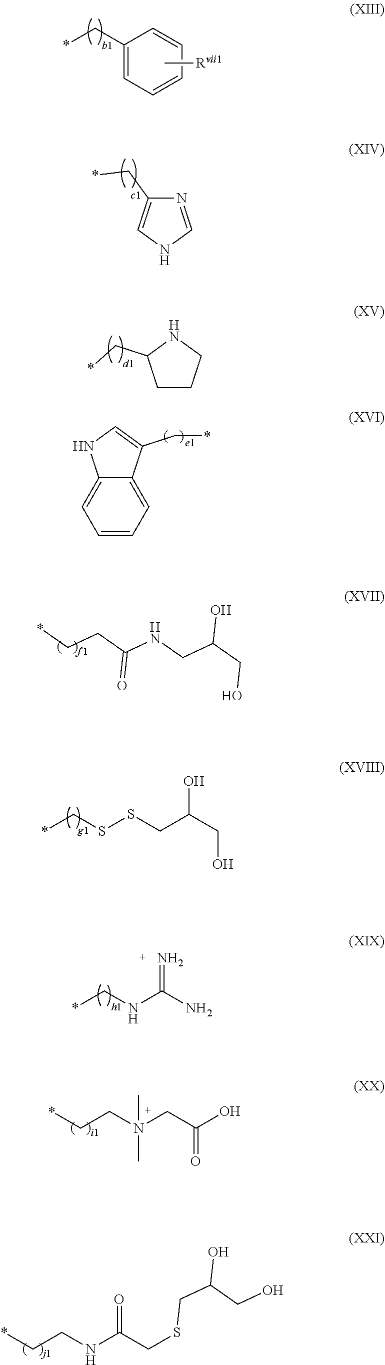

wherein “*” denotes the attaching point;
Rⁱ¹ is selected from the group consisting of H, —(C₁-C₃₀)alkyl, —(C₂-C₃₀)alkenyl, —(C₂-C₃₀)alkynyl, halogen, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NH₂, —N((C₁-C₃₀)alkyl)₂, —NH(C₁-C₃₀)alkyl, —NHC(O)—(C₁-C₃₀)alkyl, —NHC(O)O(C₁-C₃₀)alkyl, —NHC(O)NH₂, —NHC(O)N(CH₃)₂, —NHS(O)₂(C₁-C₃₀)alkyl, —NHSO₂NH₂, —C(O)(C₁-C₃₀)alkyl, —CON((C₁-C₃₀)alkyl)₂; —NO₂, —CN, —OC(O)—(C₁-C₃₀)alkyl, —OC(O)O(C₁-C₃₀)alkyl, —OC(O)NH₂, —OC(O)N((C₁-C₃₀)alkyl)₂, —SeH, —SH, —S(C₁-C₃₀)alkyl, —S(O)H, —S(O)(C₁-C₃₀)alkyl, and —SO₂(C₁-C₃₀)alkyl;
R^{vi1} is selected from the group consisting of H, —(C₁-C₃₀)alkyl, —(C₂-C₃₀)alkenyl, —(C₂-C₃₀)alkynyl, —OAlkyl(C₁-C₁₂), F, Cl, Br, I, —CF₃, —OCF₃, —NO₂, —CN, —NH₂, —(C₁-C₃₀)alkylNH₂, —N((C₁-C₃₀)alkyl)₂, and —NH(C₁-C₃₀)alkyl,
Rⁱ¹, Rⁱⁱ¹, R^{iv1} and R^{v1} are independently selected from the group consisting of H, —OH, —(C₁-C₃₀)alkyl, —(C₁-C₃₀)alkylNH₂, —(C₁-C₃₀)alkyl-N((C₁-C₃₀)alkyl)₂, and —(C₁-C₃₀)alkyl-NH(C₁-C₃₀)alkyl;
R^{vi1} is selected from the group consisting of H, —OH, —(C₁-C₃₀)alkyl, —(C₂-C₃₀)alkenyl, —(C₁-C₃₀)alkylNH₂, —NH₂, —(C₁-C₃₀)alkyl-N((C₁-C₃₀)alkyl)₂, —NH(C₂-C₃₀)alkenyl, —(C₁-C₃₀)alkyl-NH(C₁-C₃₀)alkyl, —NH-oleic, —NH-noneic, and —NH-lipoic.
wherein Rⁱ¹, Rⁱ¹, Rⁱⁱ¹, R^{iv1}, R^{v1}, R^{vi1}, and R^{vi1} are optionally substituted with one or more substituents selected from the group consisting of —OH, halogen, —O(C₁-C₃₀)alkyl, —CF₃, —OCF₃, —NH₂, —(C₁-C₃₀)alkyl, —SH, —NHNH₂, —NHCH₃, —N(CH₃)₂, —NCH(CH₃)₂ and —(C₁-C₃₀)alkyl-OH;
b1, c1, d1, e1, f1, g1, h1, i1, and j1 are integers independently ranging from 1 to 20;
wherein W1 and W2 are each independently selected from CH and N;
R₆, R₇, R₁₄, R₁₅ and R₁₉ are independently selected from the group consisting of H, —(C₁-C₃₀)alkyl, —(C₂-C₃₀)alkenyl, —(C₂-C₃₀)alkynyl, —(C₁-C₃₀)alkyl-Rⁱ², —(C₁-C₃₀)alkyl-O—Rⁱⁱ², —(C₁-C₃₀)alkyl-NR^{iv2}R^{v2}, —C(O)—R^{vi2}, and a radical selected from the group consisting of (XXII), (XXIII), (XXIV), (XXV), (XXVI), (XXVII), (XXVIII), (XXIX), (XXX), and (XXXI)

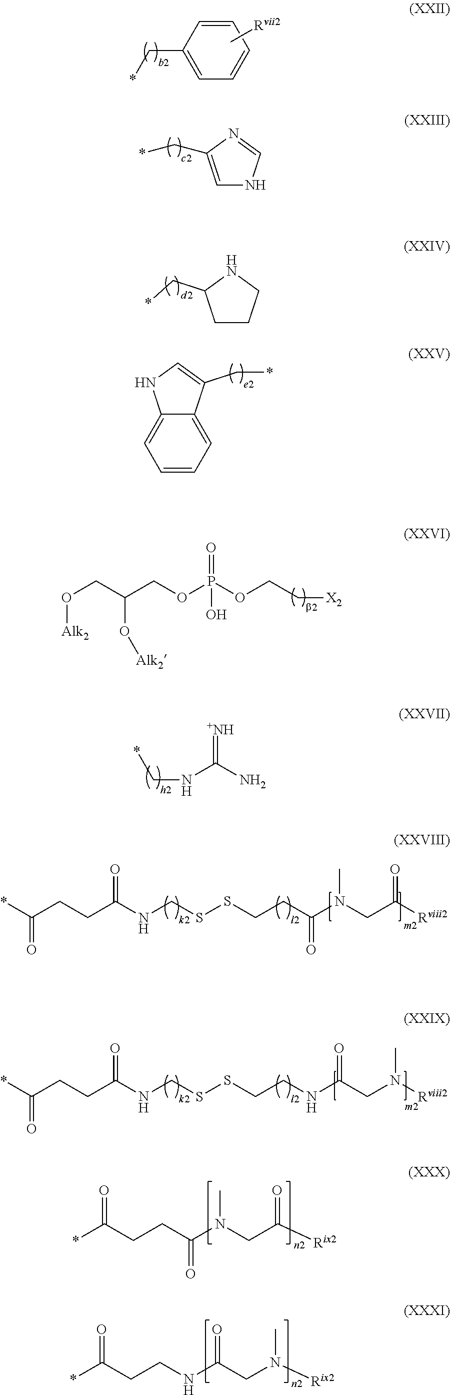

wherein “*” denotes the attaching point;
Rⁱ² is selected from the group consisting of H, —(C₁-C₃₀)alkyl, —(C₂-C₃₀)alkenyl, —(C₂-C₃₀)alkynyl, —OAlkyl(C₁-C₆), halogen, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NO₂, —CN, —OC(O)—(C₁-C₃₀)alkyl, —OC(O)O(C₁-C₃₀)alkyl, —OC(O)NH₂, —OC(O)N((C₁-C₃₀)alkyl)₂, —SH, —S(C₁-C₃₀)alkyl, —S(O)H, —S(O)(C₁-C₃₀)alkyl, and —SO₂(C₁-C₃₀)alkyl;
Rⁱⁱⁱ², R^{iv2}, and R^{v2} are independently selected from the group consisting of H, —(C₁-C₃₀)alkyl, —(C₂-C₃₀)alkenyl, —(C₁-C₃₀)alkylNH₂, —(C₁-C₃₀)alkyl-N((C₁-C₃₀)alkyl)₂, —(C₁-C₃₀)alkyl-NH(C₁-C₃₀)alkyl, and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI)

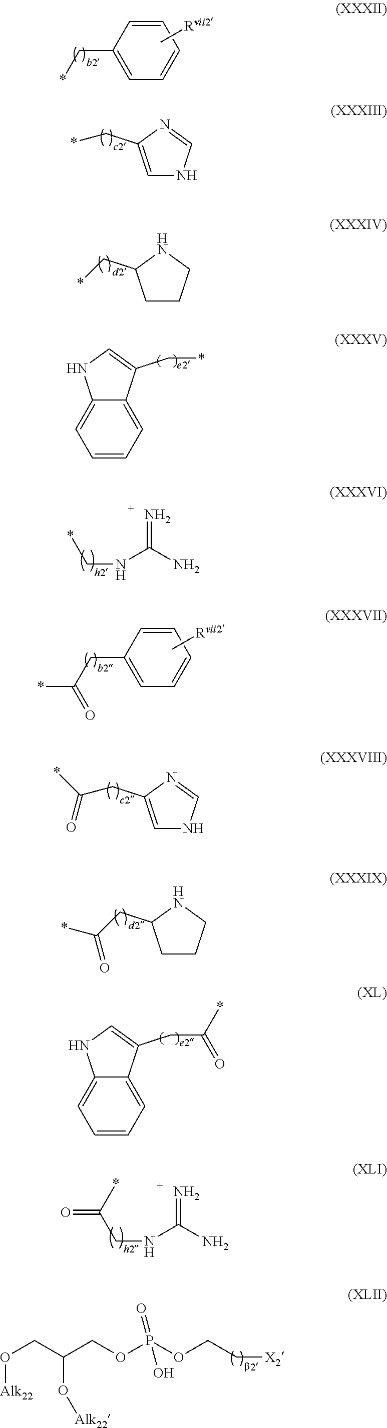

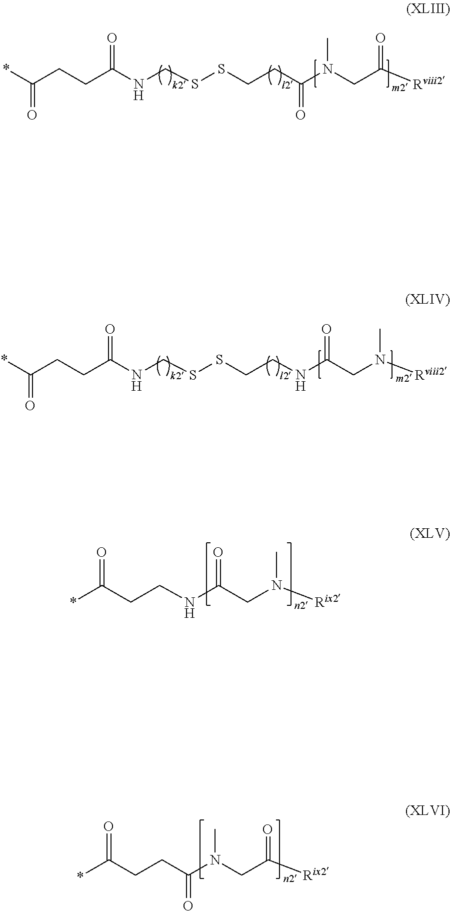

wherein R^{vii2}, R^{ix2}, R^{viii2′}, and R^{ix2″} are independently selected from the group consisting of H, —(C₁-C₁₂)alkyl, —(C₁-C₁₂)alkylNH₂, —(C₁-C₁₂)alkyl-N((C₁-C₁₂)alkyl)₂, —(C₁-C₁₂)alkyl-NH(C₁-C₁₂)alkyl, —O(C₁-C₁₂)alkyl, —COH, —CO(C₁-C₁₂)alkyl, and —O(C₂-C₃₀)alkenyl,
R^{vii2} and R^{vii2′} are independently selected from the group consisting of H, —(C₁-C₃₀)alkyl, —(C₂-C₃₀)alkenyl, —(C₂-C₃₀)alkynyl, —OAlkyl(C₁-C₁₂), F, Cl, Br, I, —CF₃, —OCF₃, —NO₂, —CN, —NH₂, —(C₁-C₃₀)alkylNH₂, —N((C₁-C₃₀)alkyl)₂, and —NH(C₁-C₃₀)alkyl,
R^{vi2} is selected from the group consisting of H, —OH, —(C₁-C₃₀)alkyl, —(C₂-C₃₀)alkenyl, —(C₁-C₃₀)alkyl-COOH, —(C₂-C₃₀)alkenyl-COOH, —(C₁-C₃₀)alkylNH₂, —NH₂, —(C₁-C₃₀)alkyl-N((C₁-C₃₀)alkyl)₂, —O—(C₁-C₃₀)alkyl, —NH(C₂-C₃₀)alkenyl, —(C₁-C₃₀)alkyl-NH(C₁-C₃₀)alkyl, —NH-oleic, —NH-noneic, —NH-lipoic, and —CH═CH(COOH)—CH₂—COOH
wherein Alk₂, Alk₂₂, Alk₂′ and Alk₂₂′ are each independently selected from the group consisting of linear or branched —(C₁-C₃₀)alkyl and linear or branched —(C₂-C₃₀)alkenyl; β₂ and β₂′ are each independently an integer from 0 to 6, and X₂ and X₂′ are each independently selected from —NH—, —COO—, and —O—;
wherein Rⁱ², Rⁱⁱⁱ², R^{vi2}, R^{v2}, R^{vi2}, R^{vii2}, R^{viii2}, R^{ix2}, R^{viii2}, and R^{ix2′} are optionally substituted by one or more substituents selected from the group consisting of H, OH, halogen, —O(C₁-C₃₀)alkyl, —CF₃, —OCF₃, —NH₂, —(C₁-C₃₀)alkyl, —(C₂-C₃₀)alkenyl, —(C₂-C₃₀)alkynyl, —SH, —NHNH₂, —NHCH₃, —N(CH₃)₂, —NCH(CH₃)₂ and —(C₁-C₅₀)alkyl-OH;
b2, c2, d2, e2, h2, k2, l2, k2′, l2′, b2″, c2″, d2″, e2″, and h2″ are integers independently ranging from 1 to 20;
b2′, c2′, d2′, e2′, and h2′ are integers independently ranging from 0 to 20;
m2, n2, m2′, and n2′ are integers independently ranging from 1 to 200;
wherein R₃, R₄, R₁₁ and R₁₃ are a biradical independently selected from the group consisting of —(C₁-C₆)alkyl-, —(C₁-C₆)alkyl-S—S—(C₁-C₆)alkyl-, —(C₁-C₆)alkyl-O—(C₁-C₆)alkyl-, and —(C₁-C₆)alkyl-NH—(C₁-C₆)alkyl-;
R₃, R₄, R₁₁ and R₁₃ are optionally substituted by one ore more substituents selected from the group consisting of —NH₂ and —(C₁-C₆)alkyl-NH₂;
with the proviso that R₃ is absent when a=1, and R₁₁ is absent when a′=1;
R₅, R₈, R₁₀, R₁₂, R₁₆ and R₁₈ are a radical independently selected from the group consisting of H and —(C₁-C₆)alkyl;
wherein R9 and R10 are optionally combined together to form a proline ring moiety;
and wherein R17 and R18 are optionally combined together to form a proline ring moiety.

As stated above, each of the repeating units defined by square brackets with their numerical value r, s, t and u respectively, i.e. PAA1, PAA2, PAA3 and PAA4, may comprise blocks of monomer units which may be the same or different from each other. Thus, the formula I as defined above, encompass compounds which may comprise repeating units defined by square brackets wherein each of the monomer units may comprise the same or different substituents. When the monomer units present in the same repeating unit PAAn are the same, the repeating unit is a “homopolymer”, whereas when the monomer units present in the same repeating unit PAAn comprise different substituents, the repeating unit is a “copolymer”, which may be a “random copolymer” or a “block copolymer”.

For the purposes of the invention, the term “homopolymer” refers to a polymer derived from a single monomer. The term “copolymer” as used herein refers to a polymer derived from more than one monomer. The copolymer may be a random or a block copolymer. The term “random copolymer” as used herein refers to a copolymer in which the monomer units are located randomly in the polymer molecule. The term “block copolymer” as used herein refers to a copolymer that comprises at least two different monomer units that upon polymerization form at least two chemically distinct regions, segments or blocks that are chemically distinguishable from one another. The term block copolymer includes linear block copolymers, multiblock copolymers and star shaped block copolymers.

A second aspect of the present disclosure relates to a conjugate comprising a compound of formula (I) as defined above, which is covalently attached to at least one labeling or imaging agent, or to at least one cell-targeting agent.

A third aspect of the present disclosure relates to a polymer complex comprising the compound of formula (I) as defined above or the conjugate according to the second aspect, and one or more active agents selected from the group consisting of pharmaceutically active agents, veterinary active agents, cosmetically active agents, nucleic acids, peptides, proteins, antibodies, aptamers, and mixtures thereof.

That at least one active agent(s) may be covalently bound directly or by one or more linkers, or alternatively that at least one active agent(s) may be non-covalently bound to the compound.

An additional aspect relates to a particle comprising the compound of formula (I) as defined herein, and optionally one or more active agents selected from the group consisting of pharmaceutically active agents, cell-targeting agents, labelling agents, imaging agents, penetration enhancing agents, cosmetically active agents, diagnostically active agents, nucleic acids, peptides, proteins, antibodies, aptamers, and mixtures thereof.

The term “non-covalent bond”, as used herein, refers to a bond that does not involve the sharing of electrons, but rather involves more dispersed variations of electromagnetic interactions between molecules. The non-covalent bond can be classified into various categories, such as electrostatic interaction, r-interaction, van der Waals forces, hydrogen bonding and hydrophobic effect.

In a preferred embodiment, at least one active agent is covalently linked to the polypeptidic backbone through an amino acid side residue via amide, ester, anhydride bonding or through a linker that includes one or more functional groups, including without limitation, alkynes, azides, reactive disulfides, maleimides, hydrazide, hydrazones, Schiff bases, acetal, aldehydes, carbamates, and reactive esters. In an alternative embodiment the covalent link is a bioresponsive one.

In another preferred embodiment, the active agent is (are) linked to the polypeptidic backbone through electrostatic interaction. Thus, anionic compounds having more negative charges than positive charges may form a polymer complex with the compounds of formula (I) when mixed in aqueous medium, through electrostatic interaction. Examples of anionic compounds include proteins, polysaccharides, lipids and nucleic acids.

The conditions for preparation, such as the aqueous medium, pH, temperature and ionic strength may be appropriately adjusted by those skilled in the art.

In accordance with a particular embodiment, the polymer complex is obtained when mixed in aqueous medium at a pH ranging from 4-9, preferably in a pH ranging from 4.5-8.5, more preferably from 5-7.5, being particularly preferred from 6.5-7.4. The pH can be easily adjusted using a buffering solution as the solvent.

In accordance with a particular embodiment, the ionic strength of the solution to be mixed can be appropriately adjusted in a range that does not destroy the structure of the nanoparticles or inhibit encapsulation of the substance to be encapsulated in the nanoparticles, and it is preferably within the range from 0-1000 mM, preferably from 0-300 mM, more preferably from 0-150 mM, being particularly preferred from 0-50 mM.

In accordance with a particular embodiment, the average molecular weight (Mw) of the compounds according to the present invention ranges from 400 Da to 500 kDa, preferably from 1 kDa to 150 kDa, more preferably from 5 kDa to 100 kDa or from 1 kDa to 50 kDa, as measured by Gel Permeation Chromatography-Refractive index-Multi Angle Light Scattering-Visible Ultraviolet (GPC-RI-MALS-UV).

In accordance with a particular embodiment, at least one active agent is selected from the group consisting of low molecular weight drugs, peptides, antibodies, hormones, enzymes, nucleic acids, proteins, and combinations thereof.

In accordance with a particular embodiment, the polymer complex (also named herein as polyplex) comprise the compound of formula (I) according to the first aspect of the disclosure, and at least a nucleic acid. In a particular embodiment, the polymer complex comprises the compound of formula (I) according to the first aspect of the disclosure, and one nucleic acid. In another particular embodiment, the polymer complex comprises the compound of formula (I) according to the first aspect of the disclosure, and a combination of two or more nucleic acids.

The compounds of formula (I) have positive charge derived from cationic groups, and hence may form a complex with the nucleic acid having negative charge through electrostatic interaction.

As used herein, the term “nucleic acid” refers to DNA or RNA. In a particular embodiment, the nucleic acid is an DNA/RNA hybrid, a short interfering RNA (siRNA), a microRNA (miRNA), a single guide RNA (sgRNA), a donorDNA, a self-amplyfing/replicating RNA, a circularRNA (oRNA), a plasmid DNA (pDNA), a closed-linear DNA (clDNA), a short hairpin RNA (shRNA), messenger RNA (mRNA), and antisense RNA (aRNA), a messenger RNA (mRNA), a CRISPR guide RNA, an antisense nucleic acid, a decoy nucleic acid, an aptamer, and a ribozyme to name a few, and encompasses both the nucleotide sequence and any structural embodiments thereof, such as double stranded, single stranded, helical, hairpin, etc, and may contain modified or unmodified bases.

When distinct nucleic acids are provided, they may be all DNA molecules or all RNA molecules or may be mixtures of DNA and RNA molecules or molecules comprising an association of DNA and RNA strands.

The nucleic acid may be a poly- or oligonucleotide, such as oligo- or poly-double stranded RNA, oligo- or poly-double stranded DNA, oligo- or poly-single stranded RNA, oligo- or poly-single stranded DNA. Each of the nucleotides contained in the nucleic acid may be a naturally occurring nucleotide or a chemically-modified, non-naturally occurring nucleotide.

The strand length of the nucleic acid is not particularly limited and the nucleic acid may have a short strand ranging from 10 to 200 bases, preferably from 20 to 180 bases, preferably from 25 to 100 bases, preferably from 30 to 50 bases; or the nucleic acid may have a relatively long strand of from 200 to 20000 bases, more preferably of from 250 to about 15000 bases.

In accordance with a particular embodiment, the nucleic acid is closed-linear DNA (clDNA), i.e. molecules wherein the double stranded region is flanked and protected by two single stranded loops thereby generating dumbbell-shaped molecules.

In a more particular embodiment, the clDNA consists of a stem region comprising a double stranded DNA sequence of interest covalently closed at both ends by hairpin loops, the clDNA comprising at least two modified nucleotides.

As used herein, the term “closed linear DNA” or “clDNA” refers to a single stranded covalently closed DNA molecule that forms a “dumbbell” or “doggy-bone” shaped structure under conditions allowing nucleotide hybridization. Therefore, although the clDNA is formed by a single stranded DNA molecule, the formation of the “dumbbell” structure by the hybridization of two complementary sequences within the same molecule generates a structure consisting on a double-stranded middle segment flanked by two single-stranded loops. The skilled in the art know how to generate clDNA from open or closed double stranded DNA using routine molecular biology techniques. For instance, those skilled in the art knows that a clDNA can be generated by attaching hairpin DNA adaptors—for instance, by the action of a ligase—to both ends of an open double stranded DNA. “Hairpin DNA adaptor” refers to a single stranded DNA that forms a stem-loop structure by the hybridization of two complementary sequences, wherein the stem region formed is closed at one end by a single stranded loop and is open at the other end.

A “modified nucleotide” is any nucleotide (e.g., adenosine, guanosine, cytidine, uracil, and thymidine) that has been chemically modified—by modification of the base, the sugar or the phosphate group—or that incorporates a non-natural moiety in its structure. Thus, the modified nucleotide may be naturally or non-naturally occurring depending on the modification.

The polymer complexes of the present disclosure constitute a useful tool for therapeutic or diagnostic indications, wherein the compounds of formula (I) as defined herein act as non-viral vectors for the delivery of the active agent resulting in an improvement of certain properties such as transfection efficiency to the desired cells, safety or toxicological profile or the release profile in physiological conditions.

The polymer complex may have a particle diameter ranging from 10 nm to 2000 nm, preferably from 20 nm to 800 nm, more preferably from 25 nm to 350 nm, from 30 nm to 300 nm, from 30 nm to 200 nm.

The polymer complex may be prepared by mixing the compound of formula (I) or the conjugate according to the second aspect of the present disclosure, and the active ingredient, in an aqueous solution buffered as required.

In accordance with some embodiments, the polymer complex is a nanoparticle, a micelle, a cylindric micelle, a reverse micelle, a vesicle or a liposome.

The compounds of the invention may be formulated in a variety of compositions, including pharmaceutical, veterinary, cosmetic and diagnostic compositions, with excipients and carriers. Thus, an additional aspect of the present disclosure relates to a composition comprising at least one conjugate or polymer complex as defined herein together with one or more pharmaceutical, veterinary, cosmetically or diagnostically acceptable excipients or carriers.

The conjugates, and polymer of the invention may be used in medicinal, cosmetic and diagnostic applications. Thus, a further aspect of the disclosure relates to the conjugates, the polymer complex or the composition of the disclosure for use as a medicament.

This aspect may also be formulated as a therapeutic product which is:
a) a conjugate as defined herein; particularly a conjugate comprising a radical derived from a compound of formula (I) as defined above, which is covalently attached to a cell-targeting agent; or alternatively,
b) a polymer complex as defined herein; or alternatively,
c) a composition as defined herein;
   for use in medicine.

This aspect of the disclosure can be reformulated as the use of the conjugate, polymer complex or the pharmaceutical composition of the disclosure for the manufacture of a medicament.

This aspect could also be formulated as a method for the treatment, diagnostics, prophylaxis and/or theranostics of a disease which comprises administering a therapeutically, diagnostically, prophylactic and/or theranostically effective amount of the polymer complex of the third aspect of the disclosure, or the pharmaceutical composition of the fifth aspect of the disclosure, together with one or more appropriate pharmaceutically, veterinary or cosmetically acceptable excipients and/or carriers, to a subject in need of it, including a human.

Another aspect of the invention relates to a diagnostic product which is a) the conjugate comprising a radical derived from a compound of formula (I) as defined above, which is covalently attached to at least one labeling or imaging agent; or alternatively, b) the diagnostic or theranostic composition as defined above, for use in diagnostics.

Another aspect of the invention relates to the use in cosmetics of a cosmetic produce which is a) the polymer complex as defined above wherein the active agent is a cosmetically active agent; or alternatively, b) the cosmetical composition as defined above.

Another aspect of the invention relates to the use of the compounds of formula (I) as defined herein, as a carrier.

In the present invention, the “subject” may be a mammal inclusive of human. The subject may be a healthy subject or a subject affected with some disease.

In the invention, “treatment” refers to curing, preventing or inducing remission of a disease or a disorder or decreasing a progressing speed of a disease or a disorder. The treatment can be attained by administering a therapeutically effective amount of a pharmaceutical composition.

When the method refers to the diagnostics, this aspect could also be formulated as a method for the diagnosis of a disease in an isolated sample of a subject, the method comprises administering to said subject an effective amount of the any of the polymer complex, or pharmaceutical composition having one or more imaging agents as defined above to the isolated sample of the subject. The detection of these imaging agents can be carried out by well-known techniques such as imaging diagnostic techniques. Examples of imaging diagnostic techniques suitable for the present disclosure include, but not limited to, are ultrasound imaging, magnetic resonance imaging (MRI), fluoroscopy, X-ray, positron emission tomography (PET), single-photon emission computed tomography (SPECT), fluorescence microscopy, and in vivo fluorescence.

Thus, the disclosure also refers to the use of the compound according to the first aspect of the disclosure, the polymer complex or the pharmaceutical composition of the disclosure as a bioimaging tool; particularly to track internalization and delivery of active agents or imaging agents.

As “bioimaging tool” is to be understood according to this description a reagent used in an imaging technique used in biology to trace some compartments of cells or particular tissues. Examples of bioimaging tools include chemiluminescent compounds, fluorescent and phosphorescent compounds, X-ray or alpha, beta, or gamma-ray emitting compounds, etc.

An additional aspect of the present disclosure relates to the use of the polymer complexes as defined herein, as non-viral vectors of general use for biomedical applications, such as vaccines or gene therapy, being effective for transfection of hosts eukaryotic cells in culture, in vivo or ex vivo, monocellular parasites and bacteria, including gene editing using the CRISP/Cas9 methodology.

In a particular embodiment, the present invention refers to the use of the polymer complexes as defined herein, as transfection reagents for delivering active agents (preferably nucleic acids regardless of size and structure, circular and linear nucleic acids) to target cells, in in vivo, in vitro or ex vivo. In a particular embodiment, the active agent is selected from the group consisting of low molecular weight drugs, peptides, proteins, antibodies, nucleic acids, aptamers, and combinations thereof.

Said transfection reagents are also useful for co-transfection of two or more active agents, e.g. two or more nucleic acids, simultaneously.

Transfection compositions (such as kits), as well as methods of using the transfection reagents to deliver nucleic acid to target cells are also within the scope of the present invention. Further embodiments will be apparent upon review of the disclosure.

The present invention also relates to a method for in vitro, ex vivo and in vivo transferring active agents comprising using a polymer complex as disclosed herein.

The present invention also relates to a method of transfecting a cell comprising contacting the cell with the polymer complex as disclosed herein.

The present invention also relates to the polymer complex or the pharmaceutical composition as defined herein, for use in a method of delivering a nucleic acid into a target cell, which comprises contacting a solution that contains the polymer complex or the pharmaceutical composition as defined herein to an animal, including human, with the target cell, so that the complex can be introduced into the target cell; transferring the complex from the endosome to the cytoplasm; dissociating the complex in the cell; and releasing the nucleic acid into the cytoplasm.

The present invention also provides compositions for use as pharmaceutical compositions for inducing a regulating effect on the expression of one or more target proteins responsible or involved in genetic hereditary diseases or complex genetic diseases, immune diseases, cancers, viral infections in various tissues/organs or tumors.

The present invention also relates to the in vitro or ex vivo use of compositions according to the invention in the production of biologics, in particular biologics encoding a recombinant protein, a peptide or an antibody; or in the production of recombinant virus, such as adeno-associated virus (AAV), lentivirus (LV), adenovirus, oncolytic virus, or baculovirus, or viral or virus-like particles, said compositions comprising a polymer complex as defined herein, comprising at least one nucleic acid molecule for transfection. As used herein, the term “biologics” refers to proteins or nucleic acids or combinations thereof, living entities such as cells or viruses, cell compartments, organoids, and tissues.

The present invention also relates to an in vitro or ex vivo use of the polymer complexes according to the invention for genome engineering, for cell reprogramming, for differentiating cells or for gene-editing.

The compositions for transfecting cells comprise a polymer complex as defined herein and an acceptable excipient, buffering agent, cell culture medium, or transfection medium.

The present invention is also directed to the compositions as defined herein for use as a therapeutic or prophylactic vaccine against viral infections, or a therapeutic vaccine against cancers. Generally, in this aspect, the vaccine is delivered through direct administration such as systemic, intramuscular, intradermal, intraperitoneal, intratumoral, oral, topical, or sub-cutaneous administration, and, in said vaccine, the composition is in association with a pharmaceutically acceptable vehicle. In other words, the vaccine can be injected directly into the body, in particular in a human individual, for inducing a cellular and/or a humoral response.

The cell targeting is achieved through different mechanisms and depends on the nature and properties of the transfection reagent, method or protocol composition or formulation and the route of administration.

In a more particular embodiment, the present invention refers to the polymer complex for use in the prevention and/or treatment of different diseases such as neurodegenerative disorders, neurological diseases, cancer, infectious diseases, disorders related to aging, neuro-inflammation, demyelinating disorder, multiple sclerosis, ischemic disorders, immune disorder, inflammatory disorders, rare diseases, among others depending on the active agent it carries.

The compounds described in the present disclosure, their pharmaceutically acceptable salts and solvates, and the pharmaceutical compositions containing them may be used jointly with other, additional drugs, to provide combined therapy. Said additional drugs may be a part of the same pharmaceutical composition or, alternatively, may be provided in the form of a separate composition for simultaneous or non-simultaneous administration with the pharmaceutical composition comprising a compound with the formula (I), a pharmaceutically acceptable salt, stereoisomer or solvate thereof.

A further aspect of the disclosure relates to the use of a compound of formula (I) as defined herein as a carrier.

An additional aspect of the disclosure relates to a device, e.g. for delivering an active agent into a cell, preferably a nucleic acid, which comprises the polymer complex of the disclosure.

This aspect may also be formulated as a device for use in a method of delivering a nucleic acid into a cell, wherein the device comprises the polymer complex as defined herein.

As will be recognized by those of ordinary skill in the art, the appropriate device for delivering an active agent into a cell will depend on the formulation of the composition or pharmaceutical composition that is selected and/or the desired administration site. For example, if the formulation of the composition is appropriate for injection in a subject, the device could be a syringe. For another example, if the desired administration site is cell culture media, the device could be a sterile pipette. For yet another example, if the desired administration site is a vein or artery, the device could be a graft. For yet another example, if the desired administration site is a subcutaneous or organ specific depot, the device could be a surgical implant.

The delivery device of the present invention may be used for a treatment (gene therapy) in which an intended nucleic acid is introduced into a cell responsible for any of various diseases.

Another aspect of the disclosure relates to a method for delivering an active agent into a target cell, preferably a nucleic acid, which comprises: administering a solution that contains the polymer complex as defined herein to an animal so that the polymer complex can be introduced into the target cell; transferring the polymer complex from the endosome to the cytoplasm; dissociating the polymer complex in the cell; and releasing the active agent into the cytoplasm.

This aspect can be reformulated as the use of the polymer complex or the pharmaceutical composition as disclosed herein, in a delivery method of a nucleic acid into a target cell, which comprises contacting a solution that contains the polymer complex or the pharmaceutical composition to an animal, including human, with the target cell, so that the complex can be introduced into the target cell; transferring the complex from the endosome to the cytoplasm; dissociating the complex in the cell; and releasing the nucleic acid into the cytoplasm.

In another aspect, the present disclosure relates to a process for the synthesis of the compound of formula (I) of the first aspect of the disclosure or any embodiment thereto, the process generally comprising polymerizing N-carboxy anhydrides (NCA) of protected or non-protected amino acids known per se, to produce a poly(amino acid) ester, and then performing aminolysis using appropriate amines. The different radicals present in the repeating units may be introduced at desired ratios by changing the ratios of the respective amines to be used at the time of the aminolysis.

In accordance with this aspect, related to a process for the synthesis of the compound of formula (I) of the first aspect of the disclosure or any embodiment thereto, the process comprising:
i) reacting an amine or tetrafluoroborate or trifluoroacetate ammonium salt form of initiator of formula (II) below
i.1) with an appropriate N-carboxyanhydride (NCA); alternatively, reacting the amine or tetrafluoroborate or trifluoroacetate ammonium salt form of initiator of step i) with an appropriate N-carboxyanhydrides in a sequential manner to obtain a block co-polymer;
i.2) alternatively, reacting the amine or tetrafluoroborate or trifluoroacetate ammonium salt form of initiator of step i) with an appropriate NCA mixture in a statistical manner to obtain random co-polymers;
ii) optionally, reacting the amine group at the N-terminal position with an amine reactive group to introduce R₁₉;
iii) optionally, orthogonally removing amino acid side chain protecting groups;
iv) optionally, reacting the amine group at side chain terminal position with an amine reactive group to introduce architectural extension, conjugation, labelling or shielding in R₇, R₈, R₁₅ or R₁₄
v) purifying the product obtained in step i), ii) or iii), optionally by fractionation, precipitation, ultrafiltration, dialysis, size-exclusion chromatography, affinity chromatography or tangential flow filtration.

Step i) above may include: a) ring opening polymerization of amino acids N-carboxyanydride (NCA) monomer by reacting the amine or tetrafluoroborate or trifluoroacetate ammonium salt form of initiator of formula (IV) above with the selected NCA, wherein the ratio monomer/initiator allows the control of the degree of polymerization (DP); b) a sequential polymerization, wherein block co-polypeptides are prepared following the polymerization reaction a) in a sequential manner, allowing the first NCA monomer to be consumed and the resulting product may be purified or not before adding the next monomer to build the following polypeptidic block; or c) a statistical polymerization a) wherein random copolypeptides are prepared following the polymerization reaction a) in a statistical manner, mixing all the NCA monomers before starting the polymerization by the addition of an amine or tetrafluoroborate or trifluoroacetate ammonium salt form of initiator.

Step ii) above corresponds to the end-capping, wherein the amine group at the N-terminal position is reacted with an amine reactive group to introduce R₁₉.

Step iii) above corresponds to the deprotection, wherein amino acid side chains are removed orthogonally depending on the protecting group.

Step iv) corresponds to the conjugation, reacting the amine group at side chain terminal position of a shielding polymer, an active small molecule, a targeting agent or an imaging agent with an amine reactive group.

Suitable amino protective groups known in the art may be used without limitation. Non-limiting examples of amino protective groups include acyl-based groups, carbamate-based groups, imide-based groups, sulfonamide-based groups, and the like. In a particular embodiment, the amino protective group is selected from the group consisting of acetyl, methyloxycarbonyl, benzyloxycarbonyl (Cbz), p-methoxybenzyloxycarbonyl, t-butyloxycarbonyl (Boc), 9-fluorenylmethyloxycarbonyl (Fmoc), allyloxycarbonyl (Alloc), 2,2,2-trichloroethoxycarbonyl group (Troc), benzoyl (Bz), benzyl (Bn), p-methoxybenzyl (PMB), 3,4-dimethoxybenzyl (DMPM), p-methoxyphenyl (PMP), tosyl (Ts), trimethylsilylethoxycarbonyl (Teoc), benzhydryl, triphenylmethyl (Trityl), (4-methoxyphenyl)diphenylmethyl (MMT), dimethoxytrityl (DMT), and diphenylphosphino, and even more particularly the amino protective group is acetyl.

Introduction and removal of amino protective groups can be carried out by standard methods such as the ones described in T. W. Green and P. G. M. Wuts, Protective Groups in Organic Chemistry, Wiley, 3rd ed. 1999, Chapter 7 (pp. 495-653).

Suitable carboxy protective groups known in the art may be used without limitation. Representative carboxy protective groups include alkyl, aryl or benzyl esters, silyl esters, amides or hydrazides. In a particular embodiment, the carboxy protective group is selected from the group consisting of —(C1-C6)alkyl, benzyl, p-methoxyphenyl, trimethylsilyl and [2-(trimethylsilyl)ethoxy]methyl (SEM).

Introduction and removal of these protective groups can be carried out by standard methods such as the ones described in T. W. Green and P. G. M. Wuts, Protective Groups in Organic Chemistry, Wiley, 3rd ed. 1999, Chapter 5 (pp. 369-451).

The term “initiator”, as used herein, refers to a chemical molecule employed for the initiation of the ring-opening polymerization (ROP) reaction of α-amino acid N-carboxyanhydrides through Normal Amine Mechanism, wherein the initiator is incorporated within the backbone of the resulting polyamino acid. The initiator may contain one or more nucleophilic groups that can initiate the ROP reaction, accordingly, the initiator may be mono- or multifunctional, respectively, resulting in one or several terminal X groups in the polymer of the invention, respectively.

The compounds of the present disclosure as defined in any of the previous embodiments or aspects of the disclosure may include isomers, depending on the presence of multiple bonds (for example, Z, E), including optical isomers or enantiomers, depending on the presence of chiral centers. In the particular case of amino acids, they may acquire L-configuration or D-configuration. Also derived of the synthetic procedure and mechanism of aminolysis, poly-Aspartic acid may also acquire α or β form isomerization of aspartamide. The individual isomers, enantiomers or diastereoisomers, and the mixtures thereof, fall within the scope of the present disclosure. The individual enantiomers or diastereoisomers, and the mixtures thereof, may be separated by means of any conventional technique well known to the person skilled in the art.

The compounds of the present disclosure may be in crystalline form as free ones or as solvates, and both forms are intended to be included within the scope of the present disclosure. In this regard, the term “solvate”, as used herein, includes both pharmaceutically acceptable solvates, i.e. solvates of the compound with the formula (I) that may be used in the preparation of a medicament, and pharmaceutically unacceptable solvates, which may be useful in the preparation of pharmaceutically acceptable solvates or salts. The nature of the pharmaceutically acceptable solvate is not critical, provided that it is pharmaceutically acceptable. In a particular embodiment, the solvate is a hydrate. The solvates may be obtained by conventional solvation methods that are well-known to persons skilled in the art. Except as otherwise specified, the compounds of the present disclosure also include compounds that differ only in the presence of one or more isotope-enriched atoms. Examples of isotope-enriched atoms, without limitation, are deuterium, tritium, 13C or 14C, or a nitrogen atom enriched in 15N.

In accordance with another aspect of the present invention, it is provided a process for preparing a compound which is structurally different from a compound of formula I as defined herein, comprising following steps:
i. using a compound of formula I as defined herein as starting compound;
ii. making structural changes to the compound of step (i) to obtain a compound, which is structurally different from a compound of formula I.

In a further aspect of the present invention, it is provided the use of a compound of formula I as defined herein, to make a compound, which is structurally different from a compound of formula I.

### BRIEF DESCRIPTION OF THE DRAWINGS

Non-limiting examples of the present disclosure will be described in the following, with reference to the appended drawings, in which:

FIG. 1 shows the result of the polyplexes PX5 (top left), PX13 (top right), PX21 (bottom left) and PX31 (bottom right) analyzed by agarose gel electrophoresis technique. This technique shows, in a qualitative manner, the ability of complexation of the polyplexes towards DNA. Also, it shows the ability to release the DNA in the presence of low and high concentration of a polyanionic competing agent (heparin) at low and high concentrations. In lane 1 the free DNA is seeded, as can be seen the free DNA is shining under the UV transiluminator. In the lanes 2,3 and 4 the polyplex is seeded at different N/P ratios (5,10 and 30) it can be seen that when the polycation is present, and the polyplex is formed, the DNA is entrapped and no signal can be observed. In lane 5, a polyplex at N/P=30 is seeded in presence of low concentration of heparin competitor showing no release in those conditions. In lane 6 the polyplex at N/P=30 is seeded together with high concentration of anionic heparin competitor, in this case, the release of the DNA is observed. This behaviour is the ideal one because the polyplexes need to be stable at low concentrations of competing molecules extracellularly but should be labile enough to release the cargo when an intracellular stimulus is applied.

### DETAILED DESCRIPTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

The term “halogen” or “halo” as used herein means fluoro, chloro, bromo, and iodo, preferably fluoro, chloro and bromo, more preferably fluoro and chloro.

The term “alkenyl”, refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. In certain instances the alkenyl group will be conjugated, in other cases an alkenyl group will not be conjugated, and yet other cases the alkenyl group may have stretches of conjugation and stretches of nonconjugation. Additionally, if there are more than 2 carbons, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted.

The term “alkyl”, refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Examples of alkyl groups having 1 to 12 carbon atoms may include methyl, ethyl, n-propyl, isopropyl, n-butyl, sec-butyl, tert-butyl, pentyl, n-hexyl, decyl and undecyl group.

Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted.

The term “alkynyl”, refers to an organic group that is comprised of carbon and hydrogen atoms that contains a triple covalent bond between two carbons. Typically, an “alkynyl” as used in this disclosure, refers to an organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C2-alkynyl can form a triple bond to a carbon of a parent chain, an alkynyl group of three or more carbons can contain more than one triple bond. Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted.

The term generally represented by the notation “Cx-Cy” (where x and y are whole integers and y>x) prior to a functional group, e.g., “C1-C12 alkyl” refers to a number range of carbon atoms. For the purposes of this disclosure any range specified by “Cx-Cy” (where x and y are whole integers and y>x) is not exclusive to the expressed range, but is inclusive of all possible ranges that include and fall within the range specified by “Cx-Cy” (where x and y are whole integers and y>x). For example, the term “C1-C4” provides express support for a range of 1 to 4 carbon atoms, but further provides implicit support for ranges encompassed by 1 to 4 carbon atoms, such as 1 to 2 carbon atoms, 1 to 3 carbon atoms, 2 to 3 carbon atoms, 2 to 4 carbon atoms, and 3 to 4 carbon atoms.

The term “moiety” refers to a specific segment or functional group of a molecule or compound.

As used herein, the term “subject” refers to any mammal, including both human and other mammals.

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. It is possible that groups in the conjugates according to the invention are substituted with one, two, three, four or five identical or different substituents, particularly with one, two or three substituents.

In the embodiments of the invention where the substitution or unsubstitution of a certain group is not specified, i.e., a certain substitution for that group is not indicated, nor is it indicated that the group is unsubstituted, it has to be understood that the possible substitution of this group is the broadest one as defined herein.

As used herein, the term “protective group” refers to a grouping of atoms that when attached to a reactive group in a molecule masks, reduces or prevents that reactivity.

Protective groups for carboxyl and amino groups are described for example in T. W. Green and P. G. M. Wuts, Protective Groups in Organic Chemistry (Wiley, 3rd ed. 1999) in Chapter 5 (pp. 369-451) and Chapter 7 (pp. 495-653), respectively.

The term “disorder” as used herein is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The term “pharmaceutically acceptable carrier,” “pharmaceutically acceptable excipient,” “physiologically acceptable carrier,” or “physiologically acceptable excipient” as used herein, refers to a pharmaceutically acceptable material, composition, or vehicle, such as a liquid or solid filler, diluent, excipient, solvent, or encapsulating material. Each component should be “pharmaceutically acceptable” in the sense of being compatible with the other ingredients of a pharmaceutical formulation. It should also be suitable for use in contact with the tissue or organ of humans and animals without excessive toxicity, irritation, allergic response, immunogenicity, or other problems or complications, commensurate with a reasonable benefit/risk ratio.

The term “cosmetically acceptable carrier” or “dermatological acceptable carrier” which is herein used interchangeably refers to that excipients or carriers suitable for use in contact with human skin without undue toxicity, incompatibility, instability, allergic response, among others.

The term “therapeutically acceptable” refers to those compounds which are suitable for use in contact with the tissues of patients without excessive toxicity, irritation, allergic response, immunogenicity, are commensurate with a reasonable benefit/risk ratio, and are effective for their intended use.

The term “pharmaceutically, cosmetically or diagnostically acceptable salts”, embraces non-toxic salts commonly used. The preparation of pharmaceutically, cosmetically or diagnostically acceptable salts of the compounds of the invention can be carried out by methods well-known in the art. Generally, such salts can be prepared by reacting the free acid or base form of a compound of the invention with a stoichiometric amount of an appropriate base or acid, respectively, in a suitable solvent such as water, an organic solvent or a mixture of them.

Examples of pharmaceutically, cosmetically or diagnostically acceptable salts include acid addition salts formed with inorganic acids e.g. hydrochloric, hydrobromic, sulfuric, nitric, hydroiodic, metaphosphoric, or phosphoric acid; and organic acids e.g. succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic and galacturonic acid; and arylsulfonic, for example benzenesulfonic, p-toluenesulfonic, oxalic, methanesulfonic or naphthalenesulfonic acid; and base addition salts formed with alkali metals and alkaline earth metals and organic bases such as N,N-dibenzylethylene-diamine, chloroprocaine, choline, diethanolamine, ethylenediamine, meglumine (N-methylglucamine), lysine and procaine; and internally formed salts. The compounds of the invention and their salts may differ in some physical properties, but they are equivalent for the purposes of the present invention.

As used herein, the term “pharmaceutically active agent” refers to and agent that has pharmacological activity and is used for curing, mitigating, treating or preventing a disease in a mammal, in particular a human. The term “cosmetic active agent” refers to an agent that does not provide any therapy but is used for aesthetic purposes, for example to improve the appearance, preserve, condition, cleanse, color or protect the skin, nails or hair.

The term “diagnostic composition” refers to a composition suitable for use in diagnostic, particularly in imaging diagnostic technology. The term “diagnostically effective amount” as used herein, refers to the effective amount of a detection polymer that, when administered, is sufficient for the diagnosis of a disease or disorder; particularly as imaging diagnostic use as contrast imaging agent. The dose of the detection polymer administered will of course be determined by the particular circumstances surrounding the case, including the polymer administered, the route of administration, the particular condition being diagnosticated, and the similar considerations. The diagnostic composition of the present invention comprises one or more diagnostically acceptable excipients or carriers. The term “diagnostically acceptable” refers to that excipients or carriers suitable for use in the diagnosing technology for preparing compositions with diagnostic use; particularly by imaging diagnostic use. The detection of these diagnostic agents in the body of the patient can be carried out by the well-known techniques used such as in imaging diagnostic with magnetic resonance imaging (MRI) and X-ray.

The terms “treat”, “treating” and “treatment”, as used herein, refers toameliorating symptoms associated with a disease or disorder, including preventing or delaying the onset of the disease or disorder symptoms, and/or lessening the severity or frequency of symptoms of the disease or disorder.

As used herein, the term “peptide” refers to molecules that comprise two or more consecutive amino acids linked to one another via peptide bonds. The term peptide includes oligopeptides and polypeptides. The term “protein” refers to large peptides, in particular peptides having at least about 50 amino acids. For the purposes of the invention, the terms peptide and protein are used interchangeably.

As used herein, the term “repeating unit”, or “block” refers to a repeating monomeric unit. A repeating unit or a block may consist of a single monomer or may be comprised of one or more monomers, randomly or block, resulting in a “mixed block”.

One skilled in the art will recognize that a monomer repeating unit is defined by square brackets (“[ ]”) depicted around the repeating monomer unit. The number (or letter representing a numerical range) on the lower right of the brackets represents the number of monomer units that are present in the polymer chain.

Only for convenience of description, in the radical of formula (II) as depicted above, the repetitive unit defined by square brackets with the numerical value r (see (1) below) is denoted as PAA1; the repetitive unit defined by square brackets with the numerical value s (see (2) below) is denoted as PAA2; the repetitive unit defined by square brackets with the numerical value t (see (3) below) is denoted as PAA3; and the repetitive unit defined by square brackets with the numerical value u (see (4) below) is denoted as PAA4

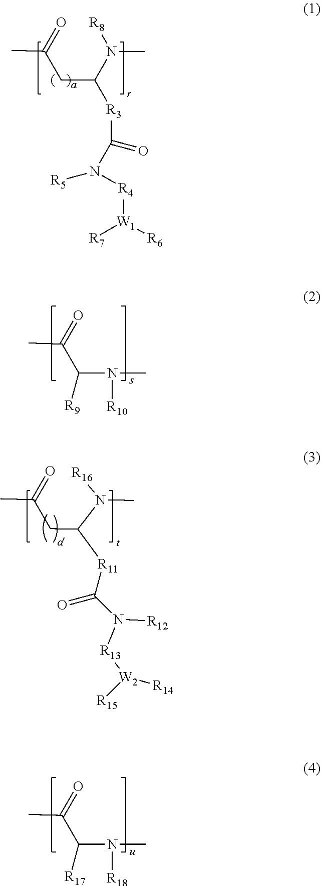

In the context of the present disclosure, the term “conjugate” refers to a polymer composite that contains the polymer compound of formula (I) and any moiety with intrinsic biological activity covalently attached to the main backbone of the polymer. In this particular context, the moiety with intrinsic biological activity may be a shielding moiety, a cell-targeting agent or a labeling or imaging agent.

In the context of the present disclosure, the term “polyplex” or “polymer complex” refers to a compound formed by electrostatic interaction between the polycationic polymer of formula I or a conjugate according to the disclosure, and any of the polianionic genetic material (preferably a nucleic acid) described above or below. The polycationic polymer of formula I or a conjugate according to the disclosure includes an opposite charge than the polianionic genetic material at the predetermined pH, resulting in the formation of multiple electrostatic bonds between the polianionic genetic material and the polymer at the predetermined pH. The driving force for the polymer complex formation is the multivalency of both polyanionic nucleic acids and polycationic polymers which results in an extremely effective entropically-driven genetic material condensation. The polymer complex (polyplex) containing a nucleic acid is useful as a nonviral synthetic vector capable of delivering the nucleic acid to a target cell. DNA or RNA delivery to a target cell mediated by a nonviral synthetic vector (e.g. a polyplex) has been widely recognized as a promising alternative method for delivery that uses a viral vector which has been confronting significant challenges and drawbacks. These include immunogenic responses (which can prevent redosing), the risk of insertional mutagenesis, the difficulty of large manufacturing at good manufacturing practice grade, limited cargo size, and cost.

The compounds of the present disclosure result in an improvement as drug carriers, or “nanovectors,” due to their inherent ability to overcome many biological barriers. Moreover, their multi-functionality permits the incorporation of cell-targeting groups, diagnostic agents, and a multitude of therapeutic agents in a single delivery system. Polymer conjugates, formed by the molecular assembly of the polymers according to the present disclosure with an imaging agent, cell-targeting groups, diagnostic agents or any other therapeutic agent, represent one notable type of multifunctional nanovector.

The compounds of the present disclosure are particularly attractive due to their ability to deliver large payloads of a variety of active ingredients (e.g. small molecule, proteins, and DNA/RNA therapeutics), their improved in vivo stability and tuneable tropism as compared to other colloidal carriers (e.g. liposomes), and their nanoscopic size which allows for passive accumulation in diseased tissues, such as solid tumors, by the enhanced permeation and retention (EPR) effect.

Using appropriate surface functionality, the compounds of the present disclosure may be further decorated with a cell-targeting group and/or permeation enhancers that can actively target cells and aid in cellular entry, resulting in a conjugate which is improved cell-specific delivery.

As used herein, the term “label or imaging” refers to a molecule that facilitates the visualization and/or detection of a targeting molecule disclosed herein.

In the context of the present disclosure the expression, “labeling or imaging agent” refers to any substance that is used as a label, or that enhances specific structures in any imaging technique. An imaging agent, hence, includes optical imaging agent, magnetic resonance imaging agent, radioisotope, and contrast agent. Imaging or labelling agents are well known in the art. Particular examples or imaging or labelling agents are gases such as sterilized air, oxygen, argon, nitrogen, fluor, perfluorocarbons, carbon dioxide, nitrogen dioxide, xenon and helium; commercially available agents used in positron emission tomography (PET), computer assisted tomography (CAT), single photon emission computerized tomography, x-ray, fluoroscopy, and magnetic resonance imaging (MRI). Examples of suitable materials for use as contrast agents in MRI include the gadolinium chelates currently available, such as diethylene triamine pentacetic acid (DTP A) and gadopentotate dimeglumine, as well as iron, magnesium, manganese, copper and chromium. Examples of materials useful for CAT and x-rays include iodine based materials for intravenous administration, such as ionic monomers typified by diatrizoate and iothalamate, non-ionic monomers such as iopamidol, isohexol, and ioversol, non-ionic dimers, such as iotrol and iodixanol, and ionic dimers, for example, ioxagalte. Other useful materials include barium for oral use and non-soluble salts such as zinc acetate. In some molecules, an imaging agent is a dye. In some molecules, an imaging agent is a fluorescent moiety. In some molecules, a fluorescent moiety is selected from: a fluorescent protein, a fluorescent peptide, a fluorescent dye, a fluorescent material or a combination thereof. Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. Examples of fluorescein dyes include, but are not limited to, 5-carboxyfluorescein, fluorescein-5-isothiocyanate, fluorescein-6-isothiocyanate and 6-carboxyfluorescein. Examples of rhodamine dyes include, but are not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®). Examples of cyanine dyes include, but are not limited to, Cy3, Cy3B, Cy3.5, Cy5, Cy5.5, Cy7, IRDYE680, Alexa Fluor 750, IRDye800CW, ICG. Examples of fluorescent peptides include GFP (Green Fluorescent Protein) or derivatives of GFP (e.g., EBFP, EBFP2, Azurite, mKalama1, ECFP, Cerulean, CyPet, YFP, Citrine, Venus, YPet). Fluorescent labels are detected by any suitable method. For example, a fluorescent label may be detected by exciting the fluorochrome with the appropriate wavelength of light and detecting the resulting fluorescence, e.g., by microscopy, visual inspection, via photographic film, by the use of electronic detectors such as charge coupled devices (CCDs), photomultipliers, etc. In some molecules, the imaging agent is labeled with a positron-emitting isotope (e.g., 18F) for positron emission tomography (PET), gamma-ray isotope (e.g., 99mTc) for single photon emission computed tomography (SPECT), or a paramagnetic molecule or nanoparticle (e.g., Gd3+ chelate or coated magnetite nanoparticle) for magnetic resonance imaging (MRI). In some molecules, the imaging agent is labeled with: a gadolinium chelate, an iron oxide particle, a super paramagnetic iron oxide particle, an ultra small paramagnetic particle, a manganese chelate or gallium containing agent. Examples of gadolinium chelates include, but are not limited to diethylene triamine pentaacetic acid (DTPA), 1,4,7,10-tetraazacyclododecane-1,4,7,10-tetraacetic acid (DOTA), and 1,4,7-triazacyclononane-N,N′,N″-triacetic acid (NOTA). In some molecules, the imaging agent is a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound. In some molecules, the imaging agent is a nuclear probe. In some molecules, the imaging agent is a SPECT or PET radionuclide probe. In some molecules, the radionuclide probe is selected from: a technetium chelate, a copper chelate, a radioactive fluorine, a radioactive iodine, a indiuim chelate. Examples of Tc chelates include, but are not limited to HYNIC, DTPA, and DOTA. In some molecules, the imaging agent contains a radioactive moiety, for example a radioactive isotope such as ²¹¹At, ¹³1I, ¹²⁵I, ⁹⁰Y, ¹⁸⁶Re, ¹⁸⁸Re, ¹⁵³Sm, ²¹²Bi, ³²P, ⁶⁴Cu radioactive isotopes of Lu, and others.

As used herein, the term “cell-targeting agent” refers to any molecule, macromolecule, or biomacromolecule displaying affinity for a (macro)molecule present in the human or animal body, which is able to direct the conjugates or the self-assembled particles thereof by directing them towards the target site for therapeutic treatment since e.g., it selectively binds to receptors that are expressed or over-expressed on specific cell types. Cell-targeting groups are well known in the art. In a more particular embodiment, the cell-targeting group is a moiety selected from galactosamine, folate, a Her-2 binding peptide, TLR agonists, β-D-Glucose, Asn-Gly-Arg peptide, angiopep2, folic acid, aptamers (A-9, A10, Anti-gp120, TTA1, sgc8, Anti MUC-1, AS1411), primaquine, zidovudine, superoxide dismutase, prednisolone, platinum, cisplatin, sulphamethoxazole, amoxicillin, etoposide, mesalzine, doxorubicin, paclitaxel, 5-amino salicylic acid, denosumab, docetaxel, calcitonin, proanthocyanidin, methotrexate, camptothecin, galactose, glycyrrheti-nic acid, lactose, hyaluornic acid, octeotride, lactobionic acid, β-galactosyl moiety, arabino-galactan, chitosan, azo-based poly-phosphazene, azo group and 4-amino-benzyl-carbamate, succinate, 4,4′-dihydroxyazo benzene-3-carboxilic acid, cyclic RGD penta-peptide, Aspartic acid octapeptide, alendronate, transferrin, bisphosphonate adendronate, mono sialoganglioside GM1, gluthatione, E-selectinthioaptamer, poloxamer-407, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptidee.g. RCPLSHSLICY), laminin receptor binding peptide (e.g. YIGSR) a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide.

The compounds of the present disclosure may be used as “non-viral vectors” (NNV) for the preparation of complexes with nucleic acids (also named as polyplexes) which result in an improvement in transfection efficiency of the nucleic acid to the desired cells or the release profile of the nucleic acid in physiological conditions.

In accordance with a more particular embodiment, the N/P ratio in the polyplexes of the disclosure, which is defined as [total number (N) of cationic groups in the block copolymer]/[total number (P) of phosphate groups in the nucleic acid] is ranging from 1 to 200, preferably from 2 to 100, more preferably from 2 to 50. The N/P ratio means a ratio between the molar concentration (N) of protonable amino groups derived from the side chain of the compound of formula (I) and the molar concentration (P) of phosphate groups derived from the nucleic acid in the mixed solution.

In a more particular embodiment, the polymer complexes defined in the third aspect of the disclosure or any embodiment thereto, may comprise an amount of the at least an active agent in the range of 1 to 80% w/w based on the mass ratio of the active agent to the conjugate. In a preferred embodiment, the range is of 1 to 70% w/w. In a still more preferred embodiment, the conjugate comprises an amount of the agent in the range of 2 to 55% w/w. Other preferred ranges are 3-30% w/w, 4-25% w/w, and 7-24% w/w.

The pharmaceutical, diagnostic or theranostic composition according to the disclosure, may be prepared in solid form or aqueous suspension, in a pharmaceutically acceptable diluent. These preparations may be administered by any appropriate administration route, for which reason said preparation will be formulated in the adequate pharmaceutical form for the selected administration route. In a more particular embodiment, administration is performed by oral, topical, rectal or parenteral route (including subcutaneous, intraperitoneal, intradermal, intramuscular, intravenous route, etc.).

In accordance with a more particular embodiment, in the compound of formula I, α, α′ and α″ are 1, thus resulting in a 6-arm star-shaped compound of formula Ia, wherein the two —NH terminal moieties of radical L are linked to the A, A′ or A″ moieties

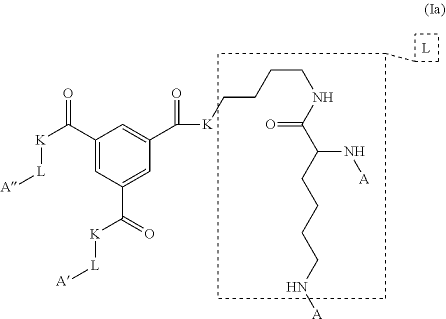

wherein K, A, A′ and A″ are as described above.

In accordance with a more particular embodiment, in the compound of formula I α, α′ and α″ are 0, thus resulting in a 3-arm star-shaped compound of formula Ib

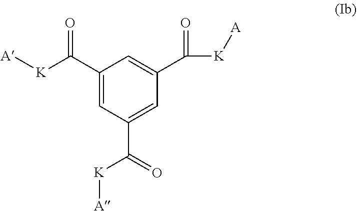

wherein K, A, A′ and A″ are as described above.

In accordance with a more particular embodiment, both for compounds of formula Ia or Ib, R1 is a biradical selected from the group consisting of

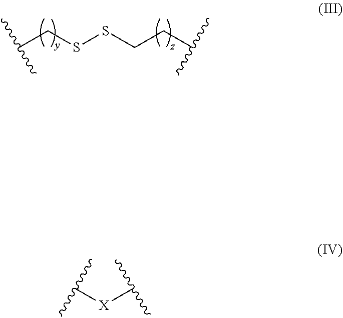

wherein the wavy lines denote the attaching points;
wherein y and z are integers independently ranging from 1 to 6;
X is a birradical selected from straight or branched —(C₁-C₁₂)alkylene-, —(C₁-C₆)alkyl-COO—, and a biradical selected from the group consisting of (V), (VI), (VII), (VIII), (IX), (X), (XI) and (XII) as defined above;
wherein the straight or branched —(C₁-C₁₂)alkylene biradical of X is optionally substituted with one or more radicals selected from the group consisting of —OH, —NRₐR_{b}, —SH, —NHNH₂, —COOR_{c}, —CF₃, —OCF₃, and halogen;
Rₐ, R_{b} and R_{c} are radicals independently selected from the group consisting of H, phenyl, —(C₁-C₁₂)alkyl, —(C₂-C₃₀)alkenyl, —(C₁-C₁₂)alkylphenyl, and -phenyl(C₁-C₁₂)alkyl.

In accordance with a more particular embodiment, both for compounds of formula Ia or Ib, R¹ is a biradical selected from the group consisting of —CH₂CH₂—S—S—CH₂CH₂—, —CH₂CH₂CH₂—S—S—CH₂CH₂CH₂—, —CH₂—, —CH₂CH₂—, —CH₂CH(CH₃)CH₂—, —CH₂CH(CH₂CH₃)CH₂—, —CH₂CH₂CH₂CH₂—, —CH₂COO—, —CH₂CH₂COO—, —CH₂CHCH₃COO—, —CH₂CH₂CH₃CH₂COO—, and a biradical selected from the group consisting of (V), (VI), (VII), (VIII), (IX), (X), (XI) and (XII) as defined above.

In accordance with a more particular embodiment, both for compounds of formula Ia or Ib,
wherein R₉ and R₁₇ are a radical independently selected from the group consisting of H, —(C₁-C₁₂)alkyl, —(C₂-C₃₀)alkenyl, —(C₁-C₁₂)alkyl-Rⁱ¹, —(C₁-C₁₂)alkyl-O—Rⁱⁱⁱ¹, —(C₁-C₁₂)alkyl-NR^{iv1}R^{v1}, —C(O)—R^{vi1}, —(C₁-C₁₂)alkyl-CO—NH₂ and a radical selected from the group consisting of (XIII), (XIV), (XV), (XVI), (XVII), (XVIII), (XIX), (XX), and (XXI) as defined above;
Rⁱ¹ is selected from the group consisting of H, F, Cl, Br, I, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NH₂, —N((C₁-C₁₂)alkyl)₂, —NH(C₁-C₁₂)alkyl, —NHC(O)—(C₁-C₁₂)alkyl, —NHC(O)O(C₁-C₁₂)alkyl, —NHC(O)NH₂, —NHC(O)N(CH₃)₂, —NHS(O)₂(C₁-C₁₂)alkyl, —NHSO₂NH₂, —SH, —S(C₁-C₁₂)alkyl, —S(O)H, —S(O)(C₁-C₁₂)alkyl, —SO₂(C₁-C₁₂)alkyl, —SeH, —C(O)(C₁-C₁₂)alkyl, and —CON((C₁-C₁₂)alkyl)₂;
R^{vii1} is selected from the group consisting of H, methyl, ethyl, propyl, isopropyl, butyl, —(C₂-C₃₀)alkenyl, —(C₂-C₃₀)alkynyl, —OAlkyl(C₁-C₁₂), F, Cl, Br, I, —CF₃, —OCF₃, —NO₂, —CN, —NH₂, —(C₁-C₆)alkylNH₂, —N((C₁-C₆)alkyl)₂, and —NH(C₁-C₆)alkyl;
Rⁱⁱ¹, R^{iv1} and R^{v1} are independently selected from the group consisting of H, —(C₁-C₁₂)alkyl, —(C₁-C₁₂)alkylNH₂, —(C₁-C₁₂)alkyl-N((C₁-C₁₂)alkyl)₂, and —(C₁-C₁₂)alkyl-NH(C₁-C₁₂)alkyl;
R^{vi1} is selected from the group consisting of H, —(C₁-C₁₂)alkyl, —(C₂-C₃₀)alkenyl, —(C₁-C₁₂)alkylNH₂, —NH₂, —(C₁-C₁₂)alkyl-N((C₁-C₁₂)alkyl)₂, —NH(C₂-C₃₀)alkenyl, —(C₁-C₁₂)alkyl-NH(C₁-C₁₂)alkyl, —NH-oleic, —NH-noneic, and —NH-lipoic;
wherein Rⁱ¹, Rⁱⁱ¹, Rⁱⁱⁱ¹, R^{iv1}, R^{v1}, R^{vi1}, and R^{vii1} are optionally substituted with one or more substituents selected from the group consisting of —OH, F, Cl, Br, I, —O(C₁-C₆)alkyl, —CF₃, —OCF₃, —NH₂, —(C₁-C₆)alkyl, —SH, —NHNH₂, —NHCH₃, —N(CH₃)₂, —NCH(CH₃)₂ and —(C₁-C₆)alkyl-OH;
b1, c1, d1, e1, f1, g1, h1, i1, and j1 are integers independently ranging from 1 to 6;
wherein R9 and R10 are optionally combined together to form a proline ring moiety;
and wherein R17 and R18 are optionally combined together to form a proline ring moiety.

In a more particular embodiment, both for compounds of formula Ia or Ib, R₉ and R₁₇ are a radical independently selected from H, methyl, ethyl, propyl, isopropyl, butyl, n-butyl, —(C₂-C₃₀)alkenyl, —CH₂SCH₃, —CH₂CH₂SCH₃, —CH₂CH₂SCH₂CH₃, —CH₂SCH₂CH₃, —CH₂SH, —CH₂—SeH, —CH₂CH₂SH, —CH₂NH₂, —CH₂CH₂NH₂, —CH₂CH₂CH₂NH₂, —CH₂CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), —C(O)H, —C(O)OCH₃, —C(O)OCH₂CH₃, —C(O)OCH(CH₃)₂, —C(O)CH₂NH₂, —C(O)CH₂CH₂NH₂, —C(O)CH(CH₃)CH₂NH₂, —C(O)CH₂NHCH₃, —C(O)CH₂NHCH₂CH₃, —C(O)CH₂CH₂NHCH₂CH₃, —C(O)CH₂CH₂CH₂NHCH₂CH₃, —C(O)CH₂CH₂CH₂NHCH₂CH₂CH₃, —C(O)CH₂CH(CH₃)CH₂NHCH₃, —C(O)CH₂CH(CH₃)CH₂NHCH₂CH₃, —C(O)CH₂CH₂NHCH₂CH₂NH₂, —C(O)CH₂CH₂CH₂NHCH₂CH₂CH₂NH₂, —C(O)CH₂CH₂N((CH(CH₃)₂)), —C(O)CH₂CH₂CH₂N((CH(CH₃)₂)), —C(O)CH₂CH₂NH(CH(CH₃)₂), —C(O)CH₂CH₂CH₂NH(CH(CH₃)₂), —CH₂COOCH₃, —CH₂CH₂COOCH₃, —CH₂C OOCH₂CH₃, —CH₂CH₂COOCH₂CH₃, —CH₂COOCH(CH₃)₂, —CH₂CONH₂, —CH₂CH₂CONH₂, —CH₂CH₂CH₂CONH₂, —CONH-oleic, —CONH-noneic, —CONH-lipoic, and a radical selected from the group consisting of (XIII), (XIV), (XV), (XVI), (XVII), (XVIII), (XIX), (XX), and (XXI) as defined above;
wherein R^{vii1} is selected from the group consisting of H, methyl, ethyl, propyl, isopropyl, butyl, —(C₂-C₃₀)alkenyl, —(C₂-C₃₀)alkynyl, —OAlkyl(C₁-C₁₂), F, Cl, Br, I, —CF₃, —OCF₃, —NO₂, —CN, —NH₂, —(C₁-C₆)alkylNH₂, —N((C₁-C₆)alkyl)₂, and —NH(C₁-C₆)alkyl; and wherein b1, c1, d1, e1, f1, g1, h1, i1, and j1 are integers independently ranging from 1 to 6;
wherein R9 and R10 are optionally combined together to form a proline ring moiety;
and wherein R17 and R18 are optionally combined together to form a proline ring moiety.

In accordance with a more particular embodiment, both for compounds of formula Ia or Ib, R₉ and R₁₇ are a radical independently selected from the group consisting of H, methyl, ethyl, propyl, isopropyl, butyl, n-butyl, —CH₂SCH₃, —CH₂CH₂SCH₃, —CH₂CH₂SCH₂CH₃, —CH₂SCH₂CH₃, —CH₂SH, —CH₂CH₂SH, —CH₂SeH, —CH₂NH₂, —CH₂CH₂NH₂, —CH₂CH₂CH₂NH₂, —CH₂CH₂CH₂CH₂NH₂, and a radical selected from the group consisting of (XIII), (XIV), (XV), (XVI), (XVII), (XVIII), (XIX), (XX), and (XXI) as defined above;
wherein R^{vii1} is selected from H, methyl, ethyl, propyl, isopropyl, butyl, —(C₂-C₃₀)alkenyl, —(C₂-C₃₀)alkynyl, —OAlkyl(C₁-C₆), F, Cl, Br, I, —CF₃, —OCF₃, —NO₂, —CN, —NH₂, —CH₂NH₂, —CH₂CH₂NH₂, —CH(CH₃)NH₂, —CH₂CH(CH₃)NH₂, —CH₂CH₂CH₂NH₂, —N(CH₃)₂, —N(CH₂CH₃)₂, —NCH(CH₃)₂, —NHCH₃, —NHCH₂CH₃, and —NHCH(CH₃)₂;
and wherein b1, c1, d1, e1, f1, g1, h1, i1, and j1 are integers independently ranging from 1 to 4;
wherein R9 and R10 are optionally combined together to form a proline ring moiety;
and wherein R17 and R18 are optionally combined together to form a proline ring moiety.

In accordance with a particular embodiment, W1 and W2 are each independently selected from CH and N, with the proviso that at least one of them is CH.

In accordance with a particular embodiment, W1 and W2 are both CH.

In accordance with a particular embodiment, W1 and W2 are both N.

In accordance with a more particular embodiment, both for compounds of formula Ia or Ib,
wherein R₆, R₇, R₁₄, R₁₅ and R₁₉ are independently selected from the group consisting of H, —(C₁-C₃₀)alkyl, —(C₂-C₃₀)alkenyl, —(C₂-C₃₀)alkynyl, —(C₁-C₃₀)alkyl-Rⁱ², —(C₁-C₃₀)alkyl-O—Rⁱⁱ², —(C₁-C₃₀)alkyl-NR^{iv2}R^{v2}, —C(O)—R^{vi2}, and a radical selected from the group consisting of (XXII), (XXIII), (XXIV), (XXV), (XXVI), (XXVII), (XXVIII), (XXIX), (XXX), and (XXXI) as defined above;
Rⁱ² is selected from the group consisting of H, —(C₁-C₁₂)alkyl, —OAlkyl(C₁-C₆), F, Cl, Br, I, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NO₂, —CN, —OC(O)—(C₁-C₁₂)alkyl, —OC(O)O(C₁-C₁₂)alkyl, —OC(O)NH₂, —OC(O)N((C₁-C₁₂)alkyl)₂, —SH, —S(C₁-C₁₂)alkyl, —S(O)H, —S(O)(C₁-C₁₂)alkyl, —SO₂(C₁-C₁₂)alkyl;

Rⁱⁱⁱ², R^{iv2}, and R^{v2} are independently selected from the group consisting of H, —(C₁-C₁₂)alkyl, —(C₂-C₃₀)alkenyl, —(C₁-C₃₀)alkylNH₂, —(C₁-C₁₂)alkyl-N((C₁-C₁₂)alkyl)₂, —(C₁-C₁₂)alkyl-NH(C₁-C₁₂)alkyl, and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI) as defined above;
wherein R^{viii2}, R^{ix2}, R^{viii2′}, and R^{ix2″} are independently selected from the group consisting of H, —(C₁-C₆)alkyl, —(C₁-C₆)alkylNH₂, —(C₁-C₆)alkyl-N((C₁-C₆)alkyl)₂, —(C₁-C₆)alkyl-NH(C₁-C₆)alkyl, —O(C₁-C₆)alkyl, —COH, —CO(C₁-C₆)alkyl, and —O(C₂-C₁₂)alkenyl,
R^{vii2} and R^{vii2} are independently selected from the group consisting of H, —(C₁-C₁₂)alkyl, —(C₂-C₃₀)alkenyl, —(C₂-C₃₀)alkynyl, —O(C₁-C₆)Alkyl, F, Cl, Br, I, —CF₃, —OCF₃, —NO₂, —CN, —NH₂, —(C₁-C₁₂)alkylNH₂, —N((C₁-C₁₂)alkyl)₂, and —NH(C₁-C₁₂)alkyl;
R^{vi2} is selected from the group consisting of H, —OH, —(C₁-C₁)alkyl, —(C₂-C₃₀)alkenyl, —(C₁-C₁₂)alkyl-COOH, —(C₂-C₃₀)alkenyl-COOH, —(C₁-C₁₂)alkylNH₂, —(C₁-C₁₂)alkyl-N((C₁-C₁₂)alkyl)₂, —O—(C₁-C₁₂)alkyl, —NH(C₂-C₁₂)alkenyl, —(C₁-C₁₂)alkyl-NH(C₁-C₁₂)alkyl, —NH-oleic, —NH-noneic, —NH-lipoic, and —CH═CH(COOH)—CH₂—COOH;
wherein Alk₂, Alk₂₂, Alk₂′ and Alk₂₂′ are each independently selected from the group consisting of linear or branched —(C₁-C₁₂)alkyl and linear or branched —(C₂-C₃₀)alkenyl; β₂ and β₂′ are each independently an integer from 0 to 6, and X₂ and X₂′ are each independently selected from the group consisting of —NH—, —COO—, and —O—;
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of OH, F, Cl, Br, I, —O(C₁-C₆)alkyl, —CF₃, —OCF₃, —NH₂, —(C₁-C₆)alkyl-NHNH₂, —NHCH₃, —N(CH₃)₂, —NCH(CH₃)₂ and —(C₁-C₆)alkyl-OH;
b2, c2, d2, e2, h2, k2, l2, k2′, l2′, b2″, c2″, d2″, e2″, and h2″ are integers independently ranging from 1 to 6;
b2′, c2′, d2′, e2′, and h2′ are integers independently ranging from 0 to 6;
m2, n2, m2′, and n2′ are integers independently ranging from 1 to 150.

In accordance with a more particular embodiment, both for compounds of formula Ia or Ib,
wherein R₆, R₇, R₁₄, R₁₅ and R₁₉ are independently selected from the group consisting of H, methyl, ethyl, propyl, isopropyl, butyl, —CH₂OCH₃, —CH₂CH₂OCH₃, —CH₂CH₂OCH₂CH₃, —CH₂CH₂OCH(CH₃)₂, —CH₂OCH₂CH₃, —CH₂OCH(CH₃)₂, —(C₂-C₃₀)alkenyl, —(C₂-C₃₀)alkynyl, —CH₂SCH₃, —CH₂CH₂SCH₃, —CH₂CH₂S CH₂CH₃, —CH₂SCH₂CH₃, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), —C(O)H, —C(O)OCH₃, —C(O)OCH₂CH₃, —C(O)OCH(CH₃)₂, —C(O)CH₂NH₂, —C(O)CH₂CH₂NH₂, —C(O)CH(CH₃)CH₂NH₂, —C(O)CH₂NHCH₃, —C(O)CH₂NHCH₂CH₃, —C(O)CH₂CH₂NHCH₂CH₃, —C(O)CH₂CH₂CH₂NHCH₂CH₃, —C(O)CH₂CH₂CH₂NHCH₂CH₂CH₃, —C(O)CH₂CH(CH₃)CH₂NHCH₃, —C(O)CH₂CH(CH₃)CH₂NHCH₂CH₃, —C(O)CH₂CH₂NHCH₂CH₂NH₂, —C(O)CH₂CH₂CH₂NHCH₂CH₂CH₂NH₂, —C(O)CH₂CH₂N((CH(CH₃)₂)), —C(O)CH₂CH₂CH₂N((CH(CH₃)₂)), —C(O)CH₂CH₂NH(CH(CH₃)₂), —C(O)CH₂CH₂CH₂NH(CH(CH₃)₂), —CH₂COOH, —CH₂CH₂COOH, —CH₂COOCH₃, —CH₂CH₂COOCH₃, —CH2COOCH₂CH₃, —CH₂CH₂COOCH₂CH₃, —CH₂COOCH(CH₃)₂, —(C₁-C₆)alkyl-Rⁱ², —(C₁-C₆)alkyl-O—Rⁱⁱ², —(C₁-C₆)alkyl-NR^{iv2}R^{v2}, —CONH-oleic, —CONH-noneic, —CONH-lipoic, and a radical selected from the group consisting of (XXII), (XXIII), (XXIV), (XXV), (XXVI), (XXVII), (XXVIII), (XXIX), (XXX), and (XXXI) as defined above;
Rⁱ² is selected from the group consisting of imidazole, N-methylpyrrole, pyrrole, pyrrolidine, pyrane, pyridine, piperidine, pyrimidine, —OC(O)NH₂, —OC(O)N((C₁-C₆)alkyl)₂;
Rⁱⁱⁱ², R^{iv2}, and R^{v2} are independently selected from the group consisting of —(C₂-C₃₀)alkenyl, —(C₂-C₃₀)alkynyl, —(C₁-C₆)alkyl-NH₂, —(C₁-C₆)alkyl-N((C₁-C₆)alkyl)₂, —(C₁-C₆)alkyl-NH(C₁-C₆)alkyl, and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI) as defined above;
R^{vii2} and R^{vii2′} are independently selected from H, methyl, ethyl, propyl, isopropyl, butyl, —OAlkyl(C₁-C₆), F, Cl, Br, I, —CF₃, —OCF₃, —NO₂, —CN, —NH₂, —(C₁-C₆)alkyl-NH₂, —N((C₁-C₆)alkyl)₂, —NH(C₁-C₆)alkyl;
wherein R^{viii2}, R^{ix2}, R^{viii2}, and R^{ix2″} are independently selected from H, —(C₁-C₆)alkyl, —(C₂-C₆)alkenyl, —(C₂-C₆)alkynyl, —(C₁-C₆)alkylNH₂, —(C₁-C₆)alkyl-N((C₁-C₆)alkyl)₂, —(C₁-C₆)alkyl-NH(C₁-C₆)alkyl;
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of OH, F, Cl, Br, —OCH₃, —OCH₂CH₃, —OCH(CH₃)₂, —CF₃, —OCF₃, —NH₂, —CH₃, —CH₂CH₃, —CH(CH₃)₂, —CH₂OH, —CH₂CH₂OH, and —CH₂CH(OH)CH₃;
b2, c2, d2, e2, h2, k2, l2, k2′, l2′, b2″, c2″, d2″, e2″, and h2″ are integers independently ranging from 1 to 4;
b2′, c2′, d2′, e2′, and h2′ are integers independently ranging from 0 to 4;
m2, n2, m2′, and n2′ are integers independently ranging from 1 to 120.

In a more particular embodiment, both for compounds of formula Ia or Ib, wherein R₁ is a biradical selected from the group consisting of —CH₂CH₂—S—S—CH₂CH₂—, —CH₂CH₂CH₂—S—S—CH₂CH₂CH₂—, —CH₂—, —CH₂CH₂—, —CH₂CH(CH₃)CH₂—, —CH₂CH(CH₂CH₃)CH₂—, —CH₂CH₂CH₂CH₂—, —CH₂COO—, —CH₂CH₂COO—, —CH₂CHCH₃COO—, —CH₂CH₂CH₃CH₂COO—, and a biradical selected from the group consisting of (V), (VI), (VII), (VIII), (IX), (X), (XI) and (XII) as defined above;
R₉ and R₁₇ are a radical independently selected from the group consisting of H, methyl, ethyl, propyl, isopropyl, butyl, n-butyl, —CH₂SCH₃, —CH₂CH₂SCH₃, —CH₂CH₂SCH₂CH₃, —CH₂SCH₂CH₃, —CH₂SH, —CH₂CH₂SH, —CH₂SeH, —CH₂NH₂, —CH₂CH₂NH₂, —CH₂CH₂CH₂NH₂, —CH₂CH₂CH₂CH₂NH₂, and a radical selected from the group consisting of (XIII), (XIV), (XV), (XVI), (XVII), (XVIII), (XIX), (XX), and (XXI) as defined above;
wherein R^{vii1} is selected from H, methyl, ethyl, propyl, isopropyl, butyl, —(C₂-C₃₀)alkenyl, —(C₂-C₃₀)alkynyl, —OCH₃, —OCH₂CH₃, F, Cl, Br, I, —CF₃, —OCF₃, —NO₂, —CN, —NH₂, —CH₂NH₂, —CH₂CH₂NH₂, —CH(CH₃)NH₂, —CH₂CH(CH₃)NH₂, —CH₂CH₂CH₂NH₂, —N(CH₃)₂, —N(CH₂CH₃)₂, —NCH(CH₃)₂, —NHCH₃, —NHCH₂CH₃, and —NHCH(CH₃)₂;
wherein b1, c1, d1, e1, f1, g1, h1, i1, and j1 are integers independently ranging from 1 to 6;
wherein R9 and R10 are optionally combined together to form a proline ring moiety;
and wherein R17 and R18 are optionally combined together to form a proline ring moiety;
wherein r, s, t and u are integers independently ranging from 0 to 250, wherein at least one of r or t is ≥1.

In accordance with a more particular embodiment, both for compounds of formula Ia or Ib,
wherein R¹ is a biradical selected from the group consisting of —CH₂CH₂—S—S—CH₂CH₂—, —CH₂CH₂CH₂—S—S—CH₂CH₂CH₂—, —CH₂—, —CH₂CH₂—, —CH₂CH(CH₃)CH₂—, —CH₂CH(CH₂CH₃)CH₂—, —CH₂CH₂CH₂CH₂—, —CH₂COO—, —CH₂CH₂COO—, —CH₂CHCH₃COO—, —CH₂CH₂CH₃CH₂COO—, and a biradical selected from the group consisting of (V), (VI), (VII), (VIII), (IX), (X), (XI) and (XII) as defined above;
R₉ and R₁₇ are a radical independently selected from H, methyl, ethyl, propyl, isopropyl, butyl, n-butyl, —CH₂SCH₃, —CH₂CH₂SCH₃, —CH₂CH₂SCH₂CH₃, —CH₂SCH₂CH₃, —CH₂SH, —CH₂CH₂SH, —CH₂SeH, —CH₂NH₂, —CH₂CH₂NH₂, —CH₂CH₂CH₂NH₂, —CH₂CH₂CH₂CH₂NH₂, and a radical selected from the group consisting of (XIII), (XIV), (XV), (XVI), (XVII), (XVIII), (XIX), (XX), and (XXI) as defined above;
R^{vii1} is selected from H, methyl, ethyl, propyl, isopropyl, butyl, —OCH₃, —OCH₂CH₃, F, Cl, Br, I, —CF₃, —OCF₃, —NO₂, —CN, —NH₂, —CH₂NH₂, —CH₂CH₂NH₂, —CH₂CH(CH₃)NH₂, —CH₂CH₂CH₂NH₂, —N(CH₃)₂, —N(CH₂CH₃)₂, —NCH(CH₃)₂, —NHCH₃, —NHCH₂CH₃, and —NHCH(CH₃)₂;
b1, c1, d1, e1, f1, g1, h1, i1, and j1 are integers independently ranging from 1 to 6;
wherein R9 and R10 are optionally combined together to form a proline ring moiety;
and wherein R17 and R18 are optionally combined together to form a proline ring moiety;
R₆, R₇, R₁₄, R₁₅ and R₁₉ are independently selected from H, methyl, ethyl, propyl, isopropyl, butyl, —CH₂OCH₃, —CH₂CH₂OCH₃, —CH₂CH₂OCH₂CH₃, —CH₂CH₂OCH(CH₃)₂, —CH₂OCH₂CH₃, —CH₂OCH(CH₃)₂, —(C₂-C₃₀)alkenyl, —(C₂-C₃₀)alkynyl, —CH₂SCH₃, —CH₂CH₂SCH₃, —CH₂SH, —CH₂SeH—CH₂CH₂SH, —CH₂CH₂SCH₂CH₃, —CH₂SCH₂CH₃, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), —C(O)H, —C(O)OCH₃, —C(O)OCH₂CH₃, —C(O)OCH(CH₃)₂, —C(O)CH₂NH₂, —C(O)CH₂CH₂NH₂, —C(O)CH(CH₃)CH₂NH₂, —C(O)CH₂NHCH₃, —C(O)CH₂NHCH₂CH₃, —C(O)CH₂CH₂NHCH₂CH₃, —C(O)CH₂CH₂CH₂NHCH₂CH₃, —C(O)CH₂CH₂CH₂NHCH₂CH₂CH₃, —C(O)CH₂CH(CH₃)CH₂NHCH₃, —C(O)CH₂CH(CH₃)CH₂NHCH₂CH₃, —C(O)CH₂CH₂NHCH₂CH₂NH₂, —C(O)CH₂CH₂CH₂NHCH₂CH₂CH₂NH₂, —C(O)CH₂CH₂N((CH(CH₃)₂)), —C(O)CH₂CH₂CH₂N((CH(CH₃)₂)), —C(O)CH₂CH₂NH(CH(CH₃)₂), —C(O)CH₂CH₂CH₂NH(CH(CH₃)₂), —CH₂COOH, —CH₂CH₂COOH, —CH₂COOCH₃, —CH₂CH₂COOCH₃, —CH₂COOCH₂CH₃, —CH₂CH₂COOCH₂CH₃, —CH₂COOCH(CH₃)₂, —(C₁-C₆)alkyl-Rⁱ², —(C₁-C₆)alkyl-O—Rⁱⁱ¹2, —(C₁-C₆)alkyl-NRi^{v2}R^{v2}, —CONH-oleic, —CONH-noneic, —CONH-lipoic, and a radical selected from the group consisting of (XXII), (XXIII), (XXIV), (XXV), (XXVI), (XXVII), (XXVIII), (XXIX), (XXX), and (XXXI) as defined above;
Rⁱ² is selected from the group consisting of imidazole, N-methylpyrrole, pyrrole, pyrrolidine, pyrane, pyridine, piperidine, pyrimidine, —OC(O)NH₂, —OC(O)N((C₁-C₆)alkyl)₂;
Rⁱⁱⁱ², R^{iv2}, and R^{v2} are independently selected from H, —(C₂-C₃₀)alkenyl, —(C₂-C₃₀)alkynyl, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI) as defined above;
wherein R^{vii2} and R^{vii2′} are independently selected from H, methyl, ethyl, propyl, isopropyl, butyl, F, Cl, Br, I, —CF₃, —OCF₃, —NO₂, —CN, —NH₂, and —(C₁-C₆)alkyl-NH₂;
wherein R^{viii2}, R^{ix2}, R^{viii2′}, and R^{ix2″} are independently selected from H, methyl, ethyl, propyl, isopropyl, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂);
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of OH, F, Cl, Br, —OCH₃, —OCH₂CH₃, —OCH(CH₃)₂, —CF₃, —OCF₃, —NH₂, —CH₃, —CH₂CH₃, —CH(CH₃)₂, —CH₂OH, —CH₂CH₂OH, and —CH₂CH(OH)CH₃;
b2, c2, d2, e2, h2, k2, l2, k2′, l2′, b2″, c2″, d2″, e2″, and h2″ are integers independently ranging from 1 to 4;
b2′, c2′, d2′, e2′, and h2′ are integers independently ranging from 0 to 4;
m2, n2, m2′, and n2′ are integers independently ranging from 1 to 120.

In accordance with a more particular embodiment, in the formula (I), r, s, t and u representing the number of repetitions of the repeating units, and are an integer ranging from 0 to 400, preferably an integer of from 0 to 250, preferably of from 0 to 200, more preferably an integer of from 0 to 150; being particularly preferred from 0 to 100.

In a more particular embodiment, r+t is an integer of from 2 to 400, preferably of from 4 to 250, preferably of from 10 to 200, preferably of from 15 to 150, preferably of from 20 to 100.

In accordance with an embodiment, in the compound of formula (I) as defined herein
a), the molar ratio of the PAA1 monomer to the PAA2 is from 100/0 to 65/35; particularly from 100/0 to 70/30, more particularly from 100/0 to 75/25, even more particularly from 100/0 to 80/20, 100/0 to 85/15, 100/0 to 90/10, 100/0 to 95/5;
b), the molar ratio of the PAA1 monomer to the PAA4 is from 100/0 to 65/35; particularly from 100/0 to 70/30, more particularly from 100/0 to 75/25, even more particularly from 100/0 to 80/20, 100/0 to 85/15, 100/0 to 90/10, 100/0 to 95/5;
c), the molar ratio of the PAA3 monomer to the PAA4 is from 100/0 to 65/35; particularly from 100/0 to 70/30, more particularly from 100/0 to 75/25, even more particularly from 100/0 to 80/20, 100/0 to 85/15, 100/0 to 90/10, 100/0 to 95/5;
d), the molar ratio of the PAA3 monomer to the PAA2 is from 100/0 to 65/35; particularly from 100/0 to 70/30, more particularly from 100/0 to 75/25, even more particularly from 100/0 to 80/20, 100/0 to 85/15, 100/0 to 90/10, 100/0 to 95/5;
and wherein the molar ratio of the sum of PAA1+PAA3 monomers to the sum of the PAA2+PAA4 is from 100/0 to 65/35; particularly from 100/0 to 70/30, more particularly from 100/0 to 75/25, even more particularly from 100/0 to 80/20, 100/0 to 85/15, 100/0 to 90/10, 100/0 to 95/5.

Particularly preferred compounds of formula I according to the disclosure are the following (details regarding to their preparation processes, complete chemical structure, ¹H NMR, DP, Mn and other characteristics are given in the examples below):

| Reference | Short name |
|---|---|
| CP5 | St-PAspDET(100) |
| CP6 | St-PAspDET(50) |
| CP7 | St-S-S-PAspDET(35) |
| CP8 | St-S-S-PAspDET(50) |
| CP9 | St-S-S-PAspDET(100) |
| CP10 | St-PAspDET(71)/DIIPA(29) |
| CP11 | St-S-S-PAspDET(45)/DIIPA(21)) |
| CP12 | St-S-S-PAspDET(78)/DIIPA(21) |
| CP13 | St-S-S-PAspDET(91)/DIIPA(29) |
| CP14 | St-S-S-PAspDET(73)/DIIPA(34) |
| CP17 | St-S-S-PAspDET(47)-co-PLeu(9) |
| CP18 | St-S-S-PAspDET(54)-co-PPhe(4) |
| CP19 | St-S-S-PAspDET(57)-co-PPhe(5) |
| CP20 | St-S-S-PAspDET(37)-co-PPhe(9) |
| CP21 | St-S-S-PAspDET(48)/DIIPA(18)-co-PLeu(4) |
| CP22 | St-S-S-PAspDET(31)/DIIPA(12)-co-PLeu(7) |
| CP23 | St-S-S-PAspDET(41)/DIIPA(10)-co-PPhe(5) |
| CP30 | St-S-S-PAspDET(42)-b-Psar(37) |
| CP31 | St-S-S-PAspDET(41)-b-Psar(22) |
| CP32 | St-S-S-PAspDET(46)-co-PLeu(8)-b-PSar(50) |
| CP33 | St-S-S-PAspDET51-co-PPhe5-b-PSar44 |
| CP34 | St-S-S-PAspDET48-co-PPhe9-b-PSar36 |
| CP35 | St-S-S-PAspDET(51)/DIIPA(20)-b-PSar(58) |
| CP36 | St-S-S-PAspDET(38)/DIIPA(21)-co-PLeu(11)-b-PSar(50) |
| CP37 | St-S-S-PAspDET(46)/DIIPA(13)-co-PPhe(11)-b-PSar(28) |
| CP38 | St-S-S-PAspDET(100)-graft into-[Succ-PSar(47)-Galactosamine]3 |
| CP39 | St-S-S-PAspDET(91)/DIIPA(29)-graft into-[Succ-PSar(23)-iPr]6 |
| CP40 | St-S-S-PAspDET(100)-graft into-[Succ-ethyl-S-S-ethyl-PSar(28)]3-COCH₃ |
| CP41 | St-S-S-PAspDET(100)-graft into-[Succ-ethyl-S-S-ethyl-PSar(28)]2-COCH₃ |
| CP42 | St-S-S-PAspDET(100)-graft into-[Succ-ethyl-S-S-ethyl-PSar(28)]1-COCH₃ |
| CP43 | St-S-S-PAspDET(100)-graft into-[Succ-ethyl-S-S-ethyl-PSar(28)]6-COCH₃ |
| CP44 | St-S-S-PAspDET(57)-co-PPhe(5)-graft into-[Succ-ethyl-S-S-ethyl-PSar(28)]2-COCH₃ |
| CP45 | St-S-S-PAspTEP(100) |
| CP46 | St-S-S-PAspImidazolamine(100) |
| CP48 | St-S-S-PAspDET(49)/Imidazolamine(17) |
| CP49 | St-S-S-PAspDET(86)/Imidazolamine(31) |
| CP50 | St-AlaVal-PAspDET(87)/PPhe(10) |
| CP51 | St-S-S-PAspDET(36)/Imidazolamine(12)-co-PPhe(5) |
| CP52 | St-S-S-PAspDET(72)-graft into-lipoic acid(5) |
| CP53 | St-S-S-PAspDET(65)/Imidazolamine(26) graft into-oleic acid(5) |
| CP54 | St-S-S-PAspDET(65)/Imidazolamine(26) graft into-oleic acid(5)/nonanoic acid(13) |
| CP55 | St-[Lys(3)]PAspDET(100) |
| CP58 | St-S-S-PAspDET(100)-b-PPro(10) |
| CP59 | St-S-S-PAspDET(119)-b-PPro(14) |

wherein the numerical values mentioned in parenthesis refer to the polymerization degree for each monomeric unit and wherein each DP value is subject to a reasonable uncertainty which is within the DP range ±20%.

It is noted that the numerical values mentioned in parenthesis refer to the degree of polymerization (DP) for each monomeric unit as a statistical number. The DP for a particular monomer unit comprised in a copolymer is calculated by a combination of two techniques. First, the ratio of different monomers is assessed by NMR spectroscopy, then the DP of each repeating structural motif is calculated based on the absolute MW given by SEC-MALS technique by dividing the molecular weight of the polymer by the molecular weight of the monomer unit. The DP of a homopolymer is directly calculated by dividing the molecular weight of the polymer by the molecular weight of the monomer unit. The DP value is reported as the central value of a gaussian distribution which comprises polymers of variable DP (depending on intrinsic polydispersity) and also this DP value is subject to a reasonable uncertainty, due to the ring-opening polymerization mechanism, which, in the context of the present invention, may be considered within the range ±20% of the nominal DP value, preferably ±15%, more preferably ±10%, even more preferably ±5%, being particularly preferred ±2%.

The term “polydispersity index” (PDI) is used as a measure of broadness of molecular weight distribution. The larger the PDI, the broader the molecular weight. PDI of a polymer is calculated as the ratio of weight average (MW) by number average (Mn) molecular weight.

$$Polydispersity index = \frac{Mw}{Mn}$$

Thus, for example, compound 35 (CP35: St-S—S-PAspDET(51)/DIIPA(20)-b-PSar(58)) is described having a PAspDET DP of 51, a DIIPA DP of 20 and PSar DP of 58; wherein the cited DP numbers are subject to a reasonable uncertainty within the ranges mentioned above.

The compounds of formula (I) described above have two or more varying amino groups and each of the amino groups show different pKa values. At pH 7.4, which is a physiological condition, the amino groups are in a partially protonated state, and hence the compound may suitably form a complex (e.g. a polyion complex) through and electrostatic interaction with a nucleic acid. When the complex is taken up into an endosome (pH 5.5), the protonation of the amino groups may further proceed to promote endosomal escape on the basis of a buffering “proton sponge” effect.

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

Throughout the description and claims the word “comprise” and variations of the word, are not intended to exclude other technical features, additives, components, or steps. Furthermore, the word “comprise” encompasses the case of “consisting of”. Additional objects, advantages and features of the disclosure will become apparent to those skilled in the art upon examination of the description or may be learned by practice of the disclosure. The following examples and drawings are provided by way of illustration, and they are not intended to be limiting of the present disclosure. Reference signs related to drawings and placed in parentheses in a claim, are solely for attempting to increase the intelligibility of the claim and shall not be construed as limiting the scope of the claim. Furthermore, the present disclosure covers all possible combinations of particular and preferred embodiments described herein.

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples, but should be determined only by a fair reading of the claims that follow.

### EXAMPLES

The following examples describe the preparation of compounds of formula I, wherein a and a′ are independently an integer from 0 to 1, and wherein R₃ is absent when a=1, and R₁₁ is absent when a′=1. A mixture of compounds wherein a=0 and a=1, and a′=0 and a′=1 may be obtained.

Only for convenience of description, in the following examples, depicted structures only represent one isomer of the aspartic backbone, i.e. when a=0 and a′=0.

Thus, for example, a compound of formula (1) is represented as follows, only for convenience of description:

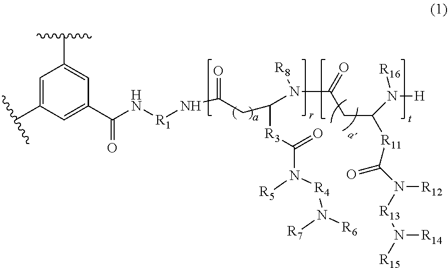

Nevertheless, the compounds of formula (1) may comprise a mixture of repeating aspartic backbone blocks as depicted in formula (1′):

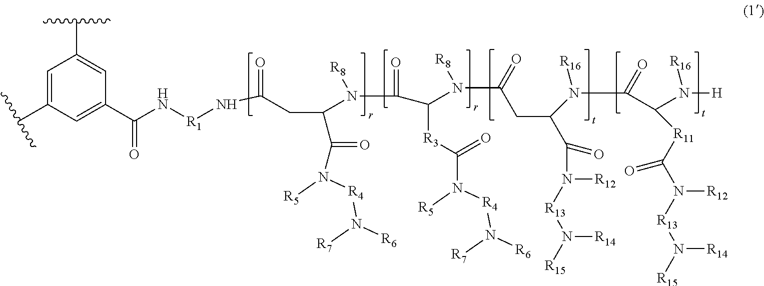

### Example 1. Preparation of Compounds of Formula (Ib1)

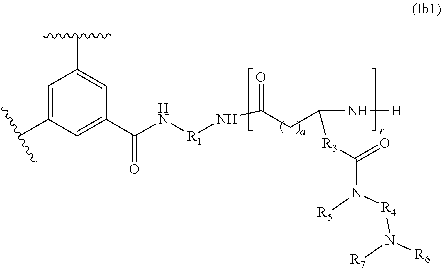

In general terms, to synthesize compounds of formula (Ib1) according to the present disclosure, first, the 3-arm star initiator was obtained within 2-3 steps. Such initiator was used to polymerize y-benzyl L-Aspartate-NCA, to yield the star polymer benzyl protected (St-PAsp(Bz)). The benzyl groups were removed by an aminolysis reaction to yield the corresponding Star-PAsp-Oligoamine.

Scheme 1 shows a particular example of polymerization and aminolysis steps:

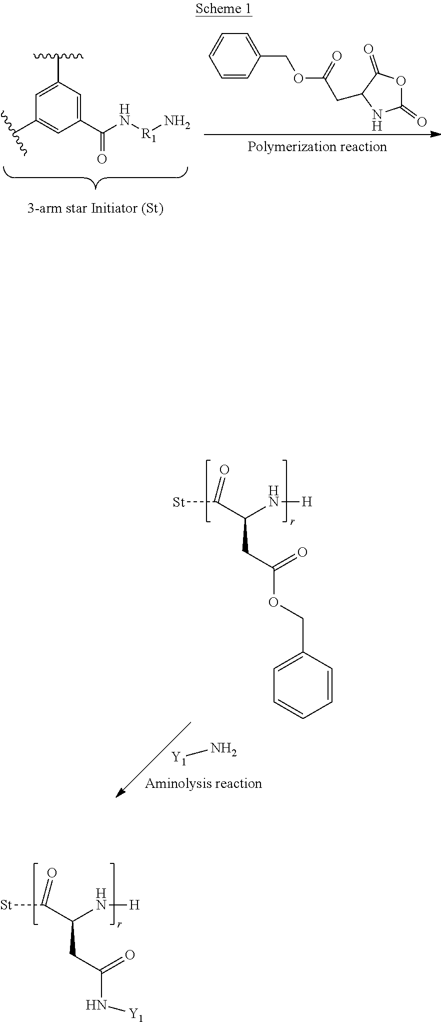

### Example 1A. Synthesis of 3-Arm Star Initiators

The synthetic routes towards two classes of 3-arm star initiators is described below.

### Example 1A.1: N¹,N³,N⁵-tris(2-aminoethyl)benzene-1,3,5-tricarboxamide (St-Initiator) (1)

The title compound was synthesized following the general procedure disclosed Scheme 2.

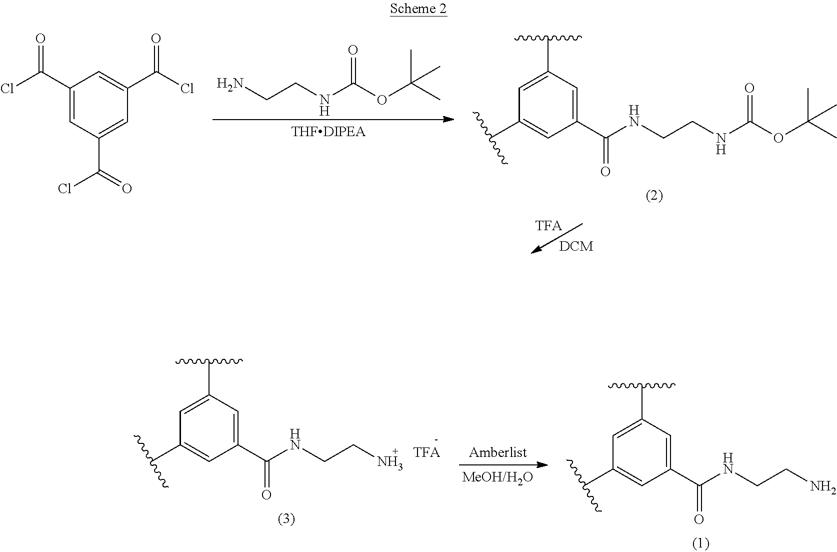 [SMILES: O=C(Cl)c1cc(C(=O)Cl)cc(C(=O)Cl)c1 ; CC(C)(C)OC(=O)NCCN ; *c1cc(*)cc(C(=O)NCCNC(=O)OC(C)(C)C)c1 ; *c1cc(*)cc(C(=O)NCCN)c1 ; *c1cc(*)cc(C(=O)NCCN)c1]

### Step (a): Synthesis of 1,3,5-tri-tert-butyl ((benzenetricarbonyltris-(azanediyl))tris(ethane-2,1-diyl))tricarbamate (2)

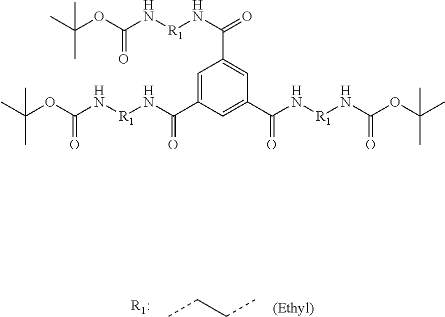

In a two-neck round bottom flask fitted with a stirrer bar, and a N₂ inlet and outlet, 500 mg of 1,3,5-benzenetricarbonyl trichloride (1.88 mmol, 1 equivalent) was dissolved in 12 mL of anhydrous THF. N,N′,N″-diisopropylethylendiamine (DIEA) (803.31 mg, 6.22 mmol, 3.3 eq.) was added to the reaction mixture followed by drop-wise addition of N-Boc-ethylendiamine (1.34 g, 6.22 mmol, 3.3 eq.) over a period of 10 min. The reaction was then left to proceed for 2 hours. After that time, the solvent was completely removed under vacuum. The product was re-dissolved in chloroform and washed 3 times with deionized water (ddH₂O), and 3 times with acidic water (pH ˜3). Finally, the organic phase was isolated under vacuum and the product was recrystallized 3 times from THF/Methanol/Hexane yielding a white crystalline solid. The product was then dried under high vacuum and stored at −20° C.

Yield: 82%. ¹H NMR (300 MHz, DMSO) δ 8.68-8.65 (m, 3H), 8.41 (s, 3H), 6.92-6.88 (m, 3H), 3.34-3.31 (m, 6H), 3.16-3.13 (m, 6H), 1.37 (s, 27H). ¹³C NMR (75 MHz, CDCl₃) δ 166.80 (C═O), 156.84 (C═O), 134.58 (Car quaternary), 128.47 (CH_{Ar}), 79.57 (C quaternary), 40.93 (CH₂), 40.43 (CH₂), 28.45 (CH₃).

### Step (b): Synthesis of 1,3,5-(benzenetricarbonyltris(azanediyl))-triethanamonium TFA Salt (3)

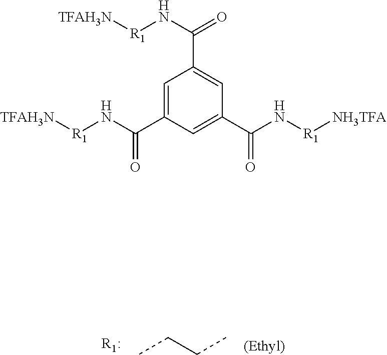

In a round bottom flask fitted with a stirrer bar and a stopper, 200 mg of 1,3,5-tri-tert-butyl ((benzenetricarbonyltris(azanediyl)) tris(ethane-2,1-diyl))tricarbamate (2) (0.33 mmol, 1 eq.) was dissolved in 5 mL of anhydrous dichlorometane and 2.5 mL of TFA was added. The reaction was stirred under nitrogen atmosphere for 2 hours and the completion of the reaction was monitored by TLC. The solvents were evaporated in vacuo. The TFA salt of the initiator (220 mg) was obtained in quantitative yield and dried under vacuum.

Yield: 98%. ¹H NMR (300 MHz, D₂O) δ 8.36 (s, 3H), 3.75 (t, J=5.9 Hz, 6H) 3.29 (t, J=6.0 Hz, 6H). ¹⁹F NMR (300 MHz, D₂O) δ −75.84.

### Step (c): Synthesis of N¹,N³,N⁵-tris(2-aminoethyl)benzene-1,3,5-tricarboxamide (1)

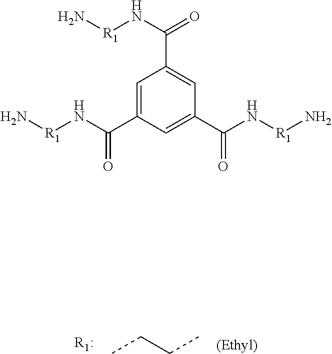

220 mg of 1,3,5-(benzenetricarbonyltris(azanediyl))triethanamonium TFA salt (3) was dissolved in 22 mL of the mixture H2O:MeOH (7:3), and stirred with an excess of the weakly basic Amberlyst ion-exchange resin (1000 mol %) for 24 hours. Then, the mixture was filtered and the filtrate was concentrated by removing the methanol. The aqueous solution was lyophilized obtaining the freebase amine in high purity.

Yield: 98%. ¹H NMR (300 MHz, D₂O) δ 8.13 (s, 3H), 3.45 (t, J=6.3 Hz, 6H) 2.86 (t, J=6.3 Hz, 6H).

### Example 1A.2: Trifluoroacetic acid salt of N,N,N-tris(2-((2-Aminoethyl) disulfanyl)ethyl)benzene-1,3,5-tricarboxamide (St-S—S-Initiator) (4)

Trifluoroacetic acid salt of N,N,N-tris(2-((2-Aminoethyl)disulfanyl)ethyl)benzene-1,3,5-tricarboxamide (St-S—S-initiator) (4) was synthesized following the general procedure disclosed in Scheme 3.

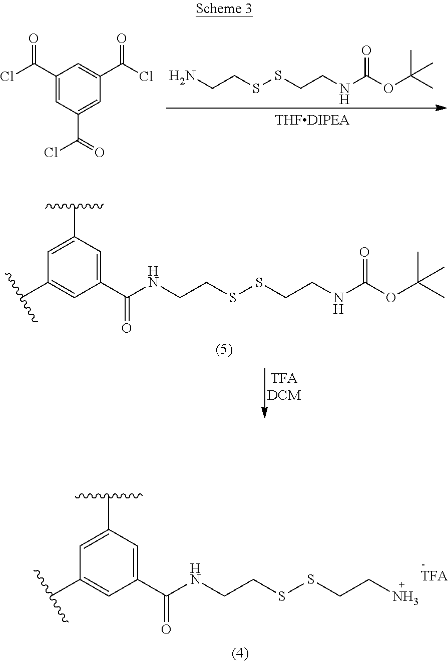 [SMILES: O=C(Cl)c1cc(C(=O)Cl)cc(C(=O)Cl)c1 ; CC(C)(C)OC(=O)NCCSSCCN ; *c1cc(*)cc(C(=O)NCCSSCCNC(=O)OC(C)(C)C)c1 ; *c1cc(*)cc(C(=O)NCCSSCCN)c1]

The synthesis of the trimeric amine initiator starts with a coupling reaction followed by amine deprotection.

### Step (a) Synthesis of tri-tert-butyl ((((benzenetricarbonyltris-(azanediyl))tris(ethane-2,1-diyl))tris(disulfanediyl))tris(ethane-2,1-diyl))-tricarbamate (5)

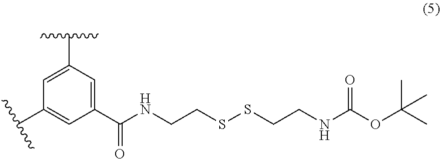 [SMILES: *c1cc(*)cc(C(=O)NCCSSCCNC(=O)OC(C)(C)C)c1]

N-(tert-Butyloxycarbonyl)cystamine (7.99, 27 mmol, 3.3 eq) was weighed into a flame-dried two-neck round-bottom flask and was dissolved in 56 mL anhydrous THF. Freshly distilled DIPEA (4.75 mL, 27 mmol, 3.3 eq) was added and stirred for 15 min at room temperature. 1,3,5-Benzenetricarbonyl trichloride (2.25 g, 8.3 mmol, 1 eq) was weighed into a flame-dried two-neck round-bottom flask and was dissolved in 28 mL of anhydrous THF. The trichloride solution was slowly added to the N-(tert-butyloxycarbonyl)cystamine mixture via syringe. The progress of reaction was monitored by thin-layer chromatography (TLC). After 4 h, the solvent was evaporated in vacuo and the residue was dissolved in ethyl acetate. The organic layer was sequentially washed with Milli-Q water, 1M hydrochloric acid and saturated sodium bicarbonate solution. The organic phase was dried over magnesium sulfate anhydrous and concentrated in vacuo afforded tri-tert-butyl ((((benzenetricarbonyltris-(azanediyl))tris(ethane-2,1-diyl))tris(disulfanediyl))tris(ethane-2,1-diyl))-tricarbamate (5) as a white foam (7.5 g, η=98%).

¹H NMR (CDCl₃): δ=1.39 (brs, 27H, —C(CH₃)₃), 2.84 (t, J=6.26 Hz, 6H, CH₂), 2.96 (t, J=6.84 Hz, 6H. CH₂), 3.46 (m, 6H, CH₂), 3.79 (m, 6H, CH₂), 5.18 (brs, 3H, —NHBoc), 7.39 (brs, 3H, aryl CH).

### Step (b): Synthesis of Trifluoroacetic Acid Salt of N,N,N-tris(2-((2-aminoethyl)disulfanyl)ethyl)benzene-1,3,5-tricarboxamide(St-S—S-Initiator) (4)

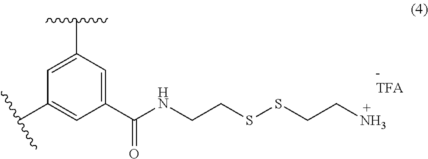 [SMILES: *c1cc(*)cc(C(=O)NCCSSCCN)c1]

7.5 g (8.19 mmol) of the initiator (5) was dissolved in anhydrous dichlorometane (180 mL) and 90 mL of TFA were added. The reaction was stirred under nitrogen atmosphere for 60 min and the completion of the reaction was monitored by TLC. The solvents were evaporated under vacuum. The TFA salt of the initiator (4) (7 g, 7.31 mmol) was obtained in quantitative yield and dried under vacuum.

¹H NMR (D₂O): δ=2.86 (m, 12H), 3.25 (t, J=6.49 Hz, 8H), 3.60 (t, J=6.85 Hz, 8H), 8.02 (brs, 3H, aryl CH).

### Example 1A.3: Trifluoroacetic Acid Salt of N1,N3,N5-tris(2-((R)-2-((R)-2-amino-3-methylbutanamido)propanamido)ethyl)benzene-1,3,5-tricarboxamide (1b)

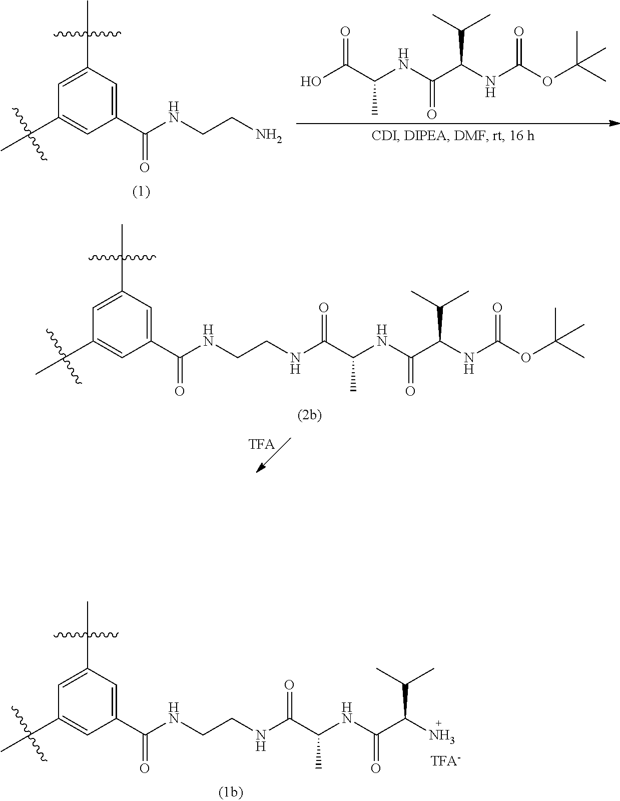 [SMILES: *c1cc(*)cc(C(=O)NCCN)c1 ; CC(C)[C@@H](NC(=O)OC(C)(C)C)C(=O)N[C@H](C)C(=O)O ; *c1cc(*)cc(C(=O)NCCNC(=O)[C@@H](C)NC(=O)[C@H](NC(=O)OC(C)(C)C)C(C)C)c1 ; *c1cc(*)cc(C(=O)NCCNC(=O)[C@@H](C)NC(=O)[C@H](N)C(C)C)c1]

The synthesis of the trimeric amine initiator starts with a coupling reaction followed by amine deprotection.

### Synthesis of tri-tert-butyl ((2,2′,2″R)-(((2,2′,2″R)-(((benzenetricarbonyltris(azanediyl))tris(ethane-2,1-diyl))tris(azanediyl))tris(1-oxopropane-2,1-diyl))tris(azanediyl))tris(3-methyl-1-oxobutane-2,1-diyl))tricarbamate. (2b)

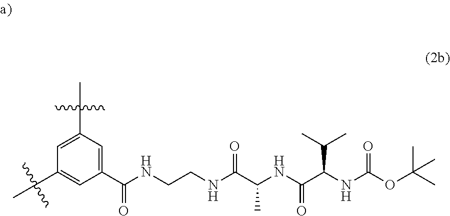 [SMILES: *c1cc(*)cc(C(=O)NCCNC(=O)[C@@H](C)NC(=O)[C@H](NC(=O)OC(C)(C)C)C(C)C)c1]

Boc-Val-Ala-OH (1.5 g, 5.35 mmol) was added to a two-necked round bottom flask fitted with a stirring bar and a stopper, then purged with 3 cycles of vacuum/N₂, and dissolved in 10 mL of DMF. Then, CDI (4 eq, 5.8 mmol, 941 mg) was added to the reaction mixture and stirred for 30 minutes at room temperature. After this time, N¹,N³,N⁵-tris(2-aminoethyl)benzene-1,3,5-tricarboxamide (St-initiator: 500 mg, 1.488 mmol) dissolved in 5 mL of DMF and DIPE (3 eq, 0.772 mL) were added. The mixture was stirred at room temperature for 16 hours. The reaction mixture dried under vacuum and the product was purified using column chromatography (Rf: 0.3, DCM:MeOH 20%) The final product was isolated as a yellow sticky solid.

The product Synthesis of tri-tert-butyl ((2,2′,2″R)-(((2,2′,2″R)-(((benzenetricarbonyltris(azanediyl))tris(ethane-2,1-diyl))tris(azanediyl))tris(1-oxopropane-2,1-diyl))tris(azanediyl))tris(3-methyl-1-oxobutane-2,1-diyl))tricarbamate presents rotamers due to the presence of carbamates in the molecule, the NMR will be described in the next step.

### Synthesis of Trifluoroacetic Acid Salt of N1,N3,N5-tris(2-((R)-2-((R)-2-amino-3-methylbutanamido)propanamido)ethyl)benzene-1,3,5-tricarboxamide (1b)

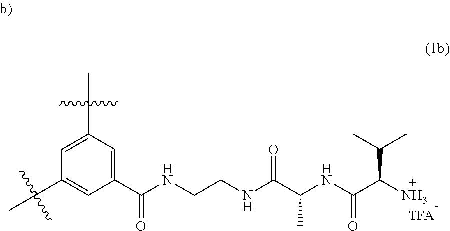 [SMILES: *c1cc(*)cc(C(=O)NCCNC(=O)[C@@H](C)NC(=O)[C@H](N)C(C)C)c1]

The experimental procedure for the acid deprotection of Boc-protecting group is the same as that described above for the trifluoroacetic acid salt of N,N,N-tris(2-((2-aminoethyl)disulfanyl)ethyl)benzene-1,3,5-tricarboxamide.

Yield: 97%. ¹H NMR (300 MHz, D₂O) δ 8.30 (s, 3H), 4.36 (q, J=7.1 Hz, CH), 3.82 (dd, J=10.7, 6.1 Hz, CH), 3.58 (dd, J=16.6, 4.6 Hz, CH₂), 2.19 (m, CH), 1.39 (dd, J=7.1, 3.9 Hz, CH₃), 0.95 (d, J=6.9 Hz, CH₃).

### Example 1A.4: N1,N3,N5-tris(2-(2,6-diaminohexanamido)ethyl)benzene-1,3,5-tricarboxamide TFA Salt

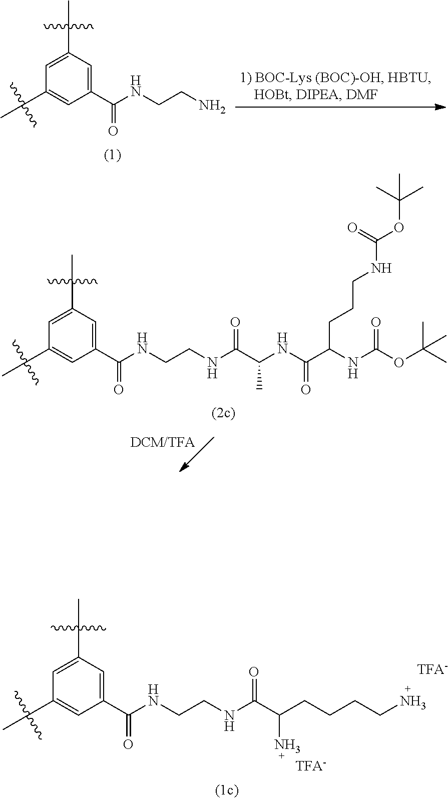 [SMILES: *c1cc(*)cc(C(=O)NCCN)c1 ; *c1cc(*)cc(C(=O)NCCNC(=O)[C@@H](C)NC(=O)C(CCCNC(=O)OC(C)(C)C)NC(=O)OC(C)(C)C)c1 ; *c1cc(*)cc(C(=O)NCCNC(=O)C(N)CCCCN)c1]

The synthesis of the trimeric amine initiator starts with a coupling reaction followed by amine deprotection.

### Synthesis of hexa-tert-butyl (((((((benzene-1,3,5-tricarbonyl)tris(azanediyl))tris(ethane-2,1-diyl))tris(azanediyl))tris(2-oxoethane-2,1-diyl))tris(azanediyl))tris(5-oxopentane-5,1,4-triyl))hexacarbamate (2c)

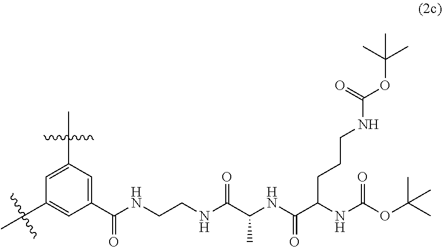 [SMILES: *c1cc(*)cc(C(=O)NCCNC(=O)[C@@H](C)NC(=O)C(CCCNC(=O)OC(C)(C)C)NC(=O)OC(C)(C)C)c1]

Boc-Lys(Boc)OH (741 mg, 2.14 mmol, 6 eq), together with N,N,N′,N′-tetramethyl-O-(1H-benzotriazol-1-yl)uranium hexafluorophosphate (HBTU, 879 mg, 2.318 mmol, 6.5 eq), and 1-hydroxybenzotriazole (HOBt, 313 mg, 2.318 mmol, 7 eq.) were weighed into a Schlenk flask and dissolved in 2 mL of DMF anh. DIPEA (617 μL, 10 eq) was directly added and the mixture was stirred for 30 minutes at 0° C. In another Schlenck flask, butyl-based 3-arm initiator (120 mg, 0.356 mmol, 1 eq) were dissolved in 1.6 mL of anhydrous DMF. The di-Boc-lysine mixture was subsequently added to the 3-arm initiator solution and stirred at room temperature for 2 days. Afterward, the reaction mixture was poured int 0.5M KHSO₄ and extracted three times with ethyl acetate. The organic layers were combined and subsequently washed with H2O and brine. After passage through anhydrous Na₂SO₄, the organic layer was evaporated under reduced pressure. The residue was purified using column chromatography (Rf=0.45, EtOAc/MeOH 10%) to yield 200 mg of the pure product.

Yield: 42%. ¹H NMR (300 MHz, D₂O) δ 8.43 (s, aryl CH), 4.02-3.91 (m, CH), 3.65-3.38 (m, CH₂), 3.05-2.96 (m, CH₂), 1.71 (m, CH₂), 1.63-1.51 (m, CH₂), 1.50-1.29 (m, CH₃).

### Synthesis of N¹,N³,N⁵-tris(2-(2,6-diaminohexanamido)ethyl)benzene-1,3,5-tricarboxamide TFA Salt

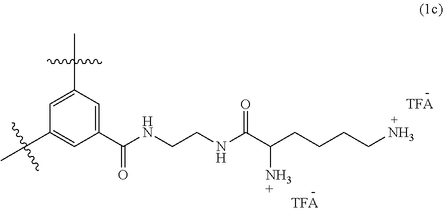 [SMILES: *c1cc(*)cc(C(=O)NCCNC(=O)C([NH3+])CCCCN)c1]

The experimental procedure for the acid deprotection of Boc-protecting group is the same as that described above for the trifluoroacetic acid salt of N,N,N-tris(2-((2-aminoethyl)disulfanyl)ethyl)benzene-1,3,5-tricarboxamide.

Yield: 98%. 1H NMR (300 MHz, D2O) δ 8.28 (s, aryl CH), 3.97 (t, J=6.6 Hz, CH), 3.68-3.44 (m, CH₂), 2.91 (t, J=7.6 Hz, CH₂), 1.98-1.78 (m, CH₂), 1.71-1.56 (m, CH₂), 1.46-1.36 (m, CH₂), 1.34 (d, J=6.4 Hz, CH₂).

### Example 1B: St-Poly(β-benzyl-L-aspartate) (Star-PAsp(Bz))(6)

General procedure for the polymerization of St-PAsp(Bz)(6) is as follows:

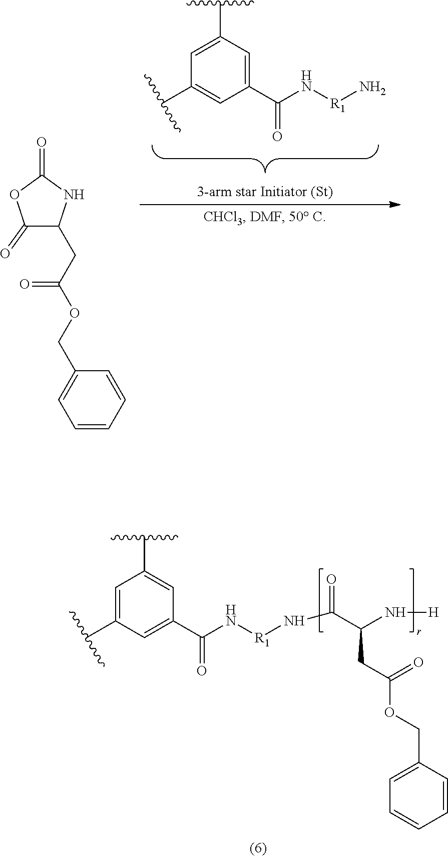

β-benzyl-L-aspartate-NCA (5 g, 2 mmol) was added to a Schlenk tube fitted with a stirrer bar, a stopper and purged with 3 cycles of vacuum/N₂, and dissolved in a mixture of anhydrous chloroform (100 mL) and DMF (6 mL). Then, the star initiator (St) was dissolved in DMF (4 mL) and was added to the reaction mixture. The mixture was stirred at 50° C. for 16 hours. Upon completion, the reaction mixture became clear and full conversion of the monomer could be detected by IR. The reaction mixture was poured into diethyl ether to precipitate the product. The precipitate was isolated by centrifugation (3750 rpm, 4 min) and dried under vacuum. St-Poly(β-benzyl-L-aspartate) (Star-PAsp(Bz)) (6) was isolated as a white solid.

Yield: 70-90% ¹H NMR (TFA): δ=2.92 (m, 2H, CH₂), 4.85 (s, 1H, CH), 5.05 (m, 2H, benzyl CH₂), 7.13 (s, 5H, aryl CH), 8.38 (s, aryl CH).

Table 1 Shows different initiators used in the polymerization processes and different DPs (degree of polymerization) obtained for different Star-PAsp(Bz) of formula (6), demonstrating the versatility and accuracy of the experimental procedure.

TABLE 1

| | | | Mn^{a} | |
|---|---|---|---|---|
| Compound | R1 | DP_{PAsp(Bz)}^{theoretical} | (kDa) | DP_{PAsp(Bz)}^{a} |
| CP1 | —CH₂CH₂— | 100 | 18.3 | 85 |
| CP2 | | 40 | 8 | 35 |
| CP3 | —CH₂CH₂SSCH₂CH₂— | 100 | 23.9 | 112 |
| CP4 | | 50 | 10.5 | 47 |

^{a}Estimated by NMR. Mn and DP refer to number average molar mass and degree of polymerization respectively.

### Example 1C. Aminolysis Reaction of poly(β-benzyl-L-aspartate) (6) to Afford St-PAsp-oligoamine (7)

General procedure for the aminolysis reaction to generate polycationic homopolymer regardless of the nature of the initiator and the amine used in the polymerization step:

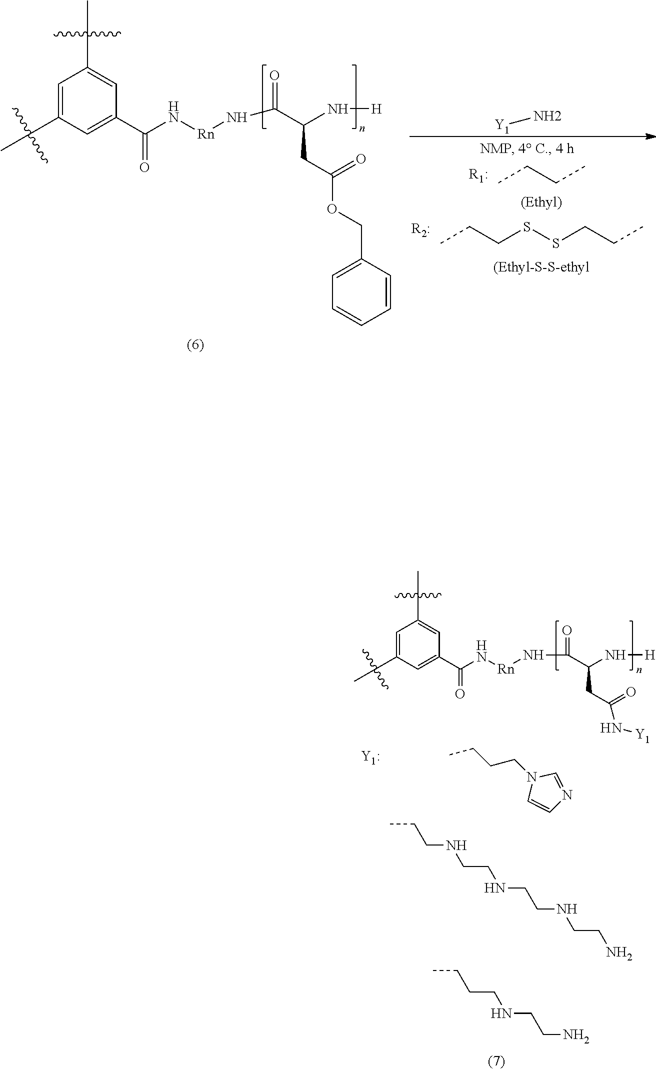

St-PAsp(Bz)(6) (DP=50, 750 mg) was dissolved in NMP (15 mL) and cooled to 4° C. This solution was added dropwise to the oligoamine (50 eq DET, TEP or imidazolamine vs unit of Asp) cooled at 4° C. and the mixture was stirred for 4 hours at the same temperature. After this time, the reaction mixture was added dropwise into cold HCl 6M for neutralization (pH 3.5). The polymer product was purified by centrifugal-assisted ultrafiltration. After filtration through 0.22 um PES filter, the remaining aqueous polymeric solution was lyophilized to obtain the final product (370 mg, q=50%).

¹H NMR [St-PAspDET](D₂O): δ 2.93 (brs, 2H, CH₂), 3.12-3.85 (m, 2H, CH₂), 8.33 (s, 3H, aryl CH).

¹H NMR [St-S—S-PAsplmidazolamine](D₂O): δ 2.02 (brs, 2H, CH₂), 2.76 (m, 2H, CH₂), 3.14 (brs, 2H, CH₂), 4.16 (m, 2H, CH₂), 4.60 (m, 1H, CH), 7.38 (s, 1H, imidazole CH), 7.43 (s, 1H, imidazole CH), 8.23 (s, 3H, aryl CH), 8.61 (m, 1H, imidazole CH).

¹H NMR [St-S—S-PAspTEP](D₂O): δ2.68-3.84 (m, 2H, CH₂), 8.38 (s, 3H, aryl CH).

Table 2 shows different St-PAsp-DET derivatives of formula (7).

TABLE 2

| | | | | Mn^{a} | Mn^{b} | | | |
|---|---|---|---|---|---|---|---|---|
| Cpd | R1 | Y₁ | DP_{Y1 theo} | (kDa) | (kDa) | DP^{a} | DP^{b} | Ð |
| CP5 | —CH₂CH₂— | DET | 100 | 24.6 | 19 | 119 | 95 | 1.112 |
| CP6 | —CH₂CH₂— | DET | 50 | 11 | 10.3 | 51 | 47 | 1.28 |
| CP7 | —CH₂CH₂SSCH₂CH₂— | DET | 35 | 11 | 6.9 | 51 | 31 | 1.305 |
| CP8 | —CH₂CH₂SSCH₂CH₂— | DET | 50 | 16.6 | 9.5 | 79 | 43 | 1.056 |
| CP9 | —CH₂CH₂SSCH₂CH₂— | DET | 100 | 22.8 | 20.3 | 110 | 97 | 1.139 |
| CP45 | —CH₂CH₂SSCH₂CH₂— | TEP | 100 | 29.5 | 39.3 | 100 | 134 | 1.04 |
| CP46 | —CH₂CH₂SSCH₂CH₂— | Imidazolamine | 100 | 31.3 | 20.8 | 137 | 89 | 1.07 |

^{a}Determined by NMR.
^{b}Determined by SEC. Mn and DP refer to number average molar mass and degree of polymerization respectively. Ð represent polydispersity as determined by SEC-MALS software analysis.

### Example 2. Synthesis of Compounds of Formula (b2)

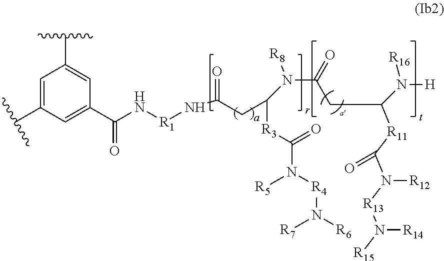

### Example 2A. Synthesis of Polycationic Polymers Star-PAspDET/(DIIPA or Imidazolamine) (8)

Star-PAspDET/(DIIPA or Imidazolamine)(8) was designed to explore how the number of amino-protonable groups would affect the toxicity and transfection efficiency, showing that the presence of a second oligoamine (DIIPA) as side-chain together with DET significantly enhances transfection efficiency without compromising toxicity. PAsp(DET/(DIIPA or Imidazolamine) was prepared by an aminolysis reaction over St-PAsp(Bz) (6) with DET and DIIPA or 1-(3aminopropyl)imidazole.

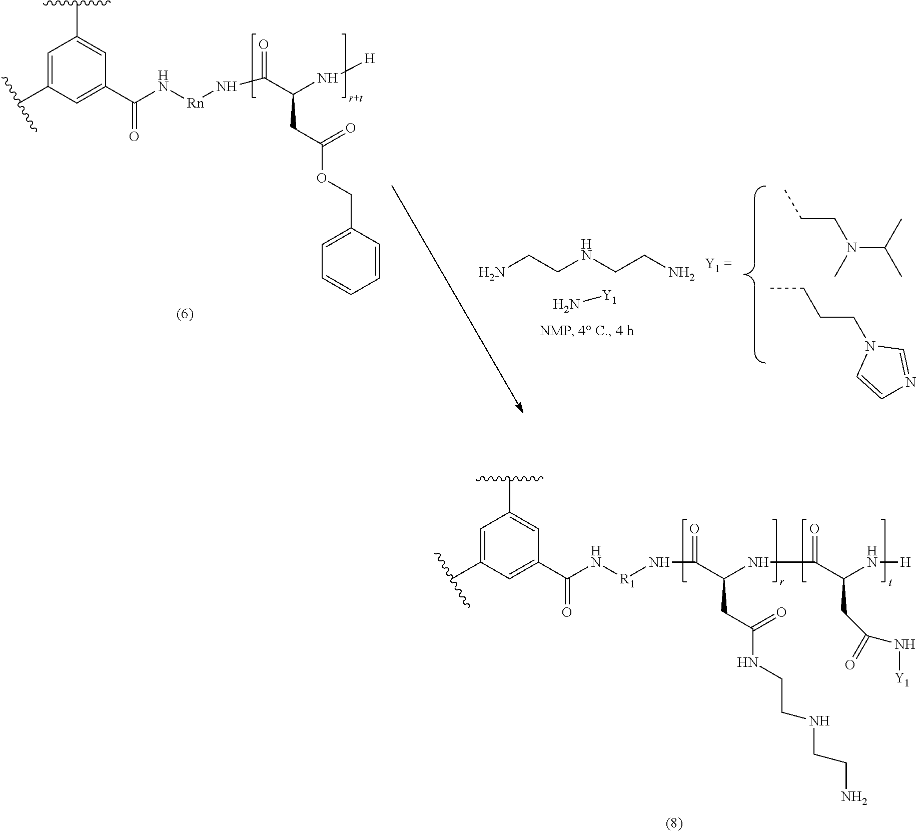

General procedure for aminolysis of St-PAspDET/(DIIPA or Imidazolamine) (8):

St-PAsp(Bz) (6) (DP=67, 60 mg) was dissolved in NMP (3 mL) and cooled to 4° C. The resultant St-PAsp(Bz) solution was added dropwise to the mixture of DET (1.58 mL) and the second primary amine (DIIIPA or Imidazolamine) (molar ratio 1:3) cooled at 4° C. and the mixture was stirred for 4 hours at the same temperature. After this time, the reaction mixture was added dropwise into cold HCl 6M for neutralization (pH 3.5). The polymer product was purified by centrifugal-assisted ultrafiltration. After filtration, the remaining aqueous polymeric solution was lyophilized to obtain the final product (η=70-60%).

¹H NMR [St-PAspDET/DIIPA](D₂O): δ=1.4 (d, J=6.4 Hz, 3H, CH₃), 2.91 (brs, 2H, CH₂), 3.15-3.88 (m, 2H, CH₂), 8.34 (s, aryl CH).

¹H NMR [St-PAspDET/Imidazolamine](D₂O): δ=2.14 (brs, 2H, CH₂), 2.87 (brs, 2H, CH₂), 3.22 (m, 2H, CH₂), 4.30 (brs, 2H, CH₂), 7.52 (s, Imidazole CH), 7.57 (s, Imidazole CH), 8.34 (s, aryl CH), 8.78 (s, Imidazole CH).

Different Star-PAspDET/(DIIPA or Imidazolamine) according of formula (8) were synthesized according to similar procedures. The introduction of ratios of the repeating unit were adjusted changing the mixing ratios of the corresponding monomer units to be allowed to react.

Table 3 shows different cationic polymers of Star-PAspDET/(DIIPA or Imidazolamine) according to formula (8)

TABLE 3

| Compound | R1 | DP^{a} | MW^{b} (KDa) | Ð |
|---|---|---|---|---|
| CP10 | —CH₂CH₂— | r(71)/t(29) | 21.2 | 1.20 |
| CP11 | —CH₂CH₂SSCH₂CH₂— | r(45)/t(21) | 15.0 | n.d |
| CP12 | —CH₂CH₂SSCH₂CH₂— | r(78)/t(21) | 20.9 | 1.04 |
| CP13 | —CH₂CH₂SSCH₂CH₂— | r(91)/t(29) | 25.7 | 1.11 |
| CP14 | —CH₂CH₂SSCH₂CH₂— | r(73)/t(34) | 23.3 | 1.07 |
| CP48 | —CH₂CH₂SSCH₂CH₂— | R(49)/t(17) | 14.6 | 1.07 |
| CP49 | —CH₂CH₂SSCH₂CH₂— | R(86)/t(31) | 24.9 | 1.05 |

^{a}Determined by NMR.
^{b}Determined by SEC.
Mn and DP refer to number average molar mass and degree of polymerization respectively.
Ð represent polydispersity as determined by SEC-MALS software analysis.

CP10 to CP14 are cationically charged polymers of Star-PAspDET/DIIPA according to formula (8), whereas CP48 and CP49 are cationically charged polymers of Star-PAspDET/Imidazolamine according to formula (8),

The proportion of DET/DIIPA or Imidazolamine(r/t) obtained in the final cationic polymer depends on the stoichiometric ratio of DET/Second primary amine with respect to St-PAsp(Bz)(6) units. For instance, for the system St-S—S-PAspDET(78)/DIIPA(21), 20 equivalents of DET and 60 equivalents of DIIPA were necessary for each unit of aspartic used (i.e. 1:3 DET/DIIPA ratio). In the case of the system St-S—S-PAspDET(91)/DIIPA(29), a 1:4 DET/DIIPA ratio was necessary. For the system St-S—S-PAspDET(49)/Imidazolamine(17) and St-S—S-PAspDET(86)/Imidazolamine(31), 20 equivalents of DET and 40 equivalents of 1-(3aminopropyl)imidazole were necessary for 66 and 100 units of aspartic.

### Example 3. Synthesis of the Amphiphilic Block Copolymer with Formula (1b3)

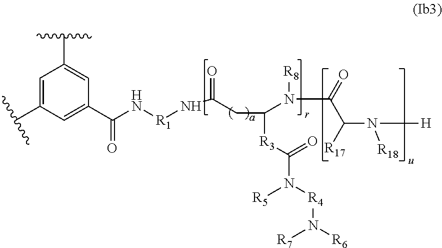

The presence of hydrophobic groups in the copolymer generates amphiphilic systems enabling to increase the efficiency of transfection in cells.

Bellow is the synthetic route for the introduction of an amphiphilic copolymer. In particular, the example discloses the preparation of St-Rn-PAsp(DET)-co-PLeu and St-S—S-PAsp(DET)-co-PPhe

### Example 3A. Synthesis of Star-PAsp(Bz) (9) Copolymers Comprising a Hydrophobic Fragment

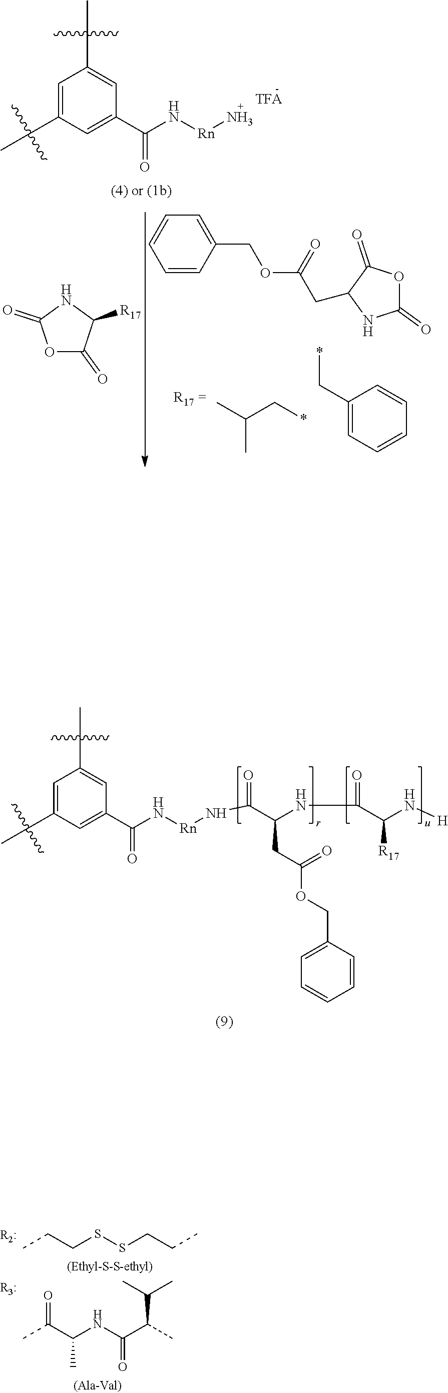

To synthesize the copolymers with hydrophobic residues, the polymerization was carried out via ring opening polymerization mechanism using trifluoroacetic acid salt of N,N,N-tris(2-((2-aminoethyl)disulfanyl)ethyl)-benzene-1,3,5-tricarboxamide or Trifluoroacetic acid salt of N1,N3,N5-tris(2-((R)-2-((R)-2-amino-3-methylbutanamido)propanamido)ethyl)benzene-1,3,5-tricarboxamide as initiator.

### General Procedure for Synthesized St-S—S-PAsp(Bz)(45)-Co-PLeu(5):

β-benzyl-L-aspartate-N-carboxy anhydride (3.5 g, 14.15 mmol) and L-Leucine-N-carboxy anhydride (247 mg, 1.57 mmol) was added to a Schlenk tube fitted with a stirrer bar and a stopper, and purged with 3 cycles of vacuum/N₂, and dissolved in a mixture of anhydrous chloroform (100 mL) and DMF (6 mL). Then, the star initiator was dissolved in DMF (4 mL) and was added to the reaction mixture. The mixture was stirred at 50° C. for 16 hours. Upon completion, the reaction mixture became clear and full conversion of the monomer could be detected by IR. The reaction mixture was poured into diethyl ether to precipitate the product. The precipitate was isolated by centrifugation (3750 rpm, 4 min) and dried under vacuum. Copolymer was isolated as a white solid.

Yield: 70-90% 1H NMR (TFA): δ=0.95 (s, 3H, CH₃), 3.05 (s, 2H, CH), 4.98 (brs, 1H, CH), 5.17 (m, 2H, benzyl CH₂), 7.26 (s, 5H, aryl CH), 8.51 (s, aryl CH).

Different St-Rn-PAsp(Bz)-co-PLeu according of formula (9) were synthesized according to similar procedures. The introduction of ratios of the repeating unit were adjusted changing the mixing ratios of the corresponding monomer units to be allowed to react.

Table 4 shows the ratio of copolymer St-Rn-PAsp(Bz)-co-PLeu according to formula (9).

TABLE 4

| Compound | R17 | DP_{PAsp(Bz)}^{theo} | DP_{R17}^{theo} | DP_{PAsp(Bz)} ^{a} | DP_{R17}^{a} |
|---|---|---|---|---|---|
| CP15 | Leu | 45 | 5 | 63 | 4 |
| CP16 | Leu | 40 | 10 | 59 | 11 |

^{a}Determined by NMR.

In the table above, R₁₇=Leu refers to a Leucine side chain. And Rn=—CH₂CH₂SSCH₂CH₂—

An analog system was synthetized by substituting the leucine block for phenylalanine. However, the hydrophobic residue matches by 1H-NMR with the protective group of the poly-aspartic. These system will be analyzed after the aminolysis reaction.

The same experimental procedure is performed for the synthesis of St-Ala-Val-PAspDET-co-PPhe as described above but in this case using Trifluoroacetic acid salt of N1,N3,N5-tris(2-((R)-2-((R)-2-amino-3-methylbutanamido)propanamido)ethyl)benzene-1,3,5-tricarboxamide as initiator. These system will be analyzed after the aminolysis reaction.

### Example 3B. Synthesis of Amphiphilic Polyaspartamide Derivatives St-Rn-PAspDET-co-PR₁₇ (10)

As shown in the synthetic route below, various St-Rn-PAspDET-co-PR₁₇ (10) serving as amphiphilic polyamino acids were prepared by the simultaneous aminolysis reaction of PBLA with DET.

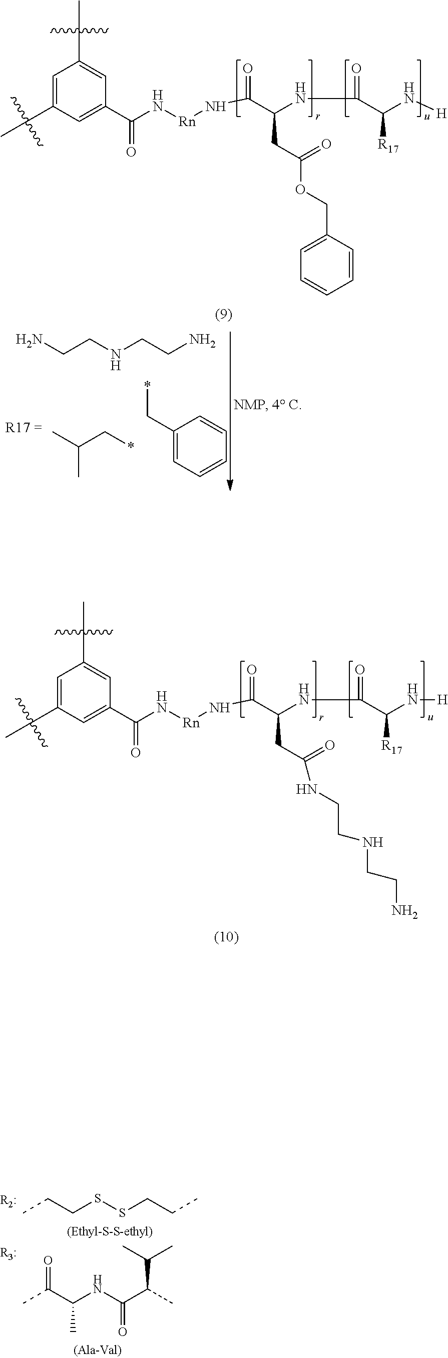

As an example, herein we describe the synthetic method in which R₁₇ represents either a leucine or a phenylalanine group. Copolymer of St-S—S-PAsp(Bz)45-co-PLeu(5) (500 mg of copolymer, 470 mg of PBLA, DP:45) was dissolved in NMP (10 mL) and cooled to 4° C. The resultant copolymer solution was added dropwise to the mixture of DET (12 mL, 50 eq. vs unit of PAsp(Bz)) and the solution was stirred for 4 h at 4° C. under nitrogen atmosphere. After this time, the reaction mixture was added dropwise into cold HCl 6 M for neutralization (pH 3.5). The polymer product was purified by centrifugal-assisted ultrafiltration. After filtration, the remaining aqueous polymeric solution was lyophilized to obtain the final product.

Yield: 65-80%. ¹H NMR (D₂O) [Rn=—CH₂CH₂SSCH₂CH₂—, R₁₇=Leu side chain]: 6=0.95 (d, J=20.0 Hz, 3H, CH₃), 1.64 (brs, 1H, CH), 3.07-2.77 (m, 2H, CH₂), 3.79-3.14 (m, 8H, CH₂), 8.33 (s, aryl CH).

Yield: 70-80%. ¹H NMR (D₂O) [Rn=—CH₂CH₂SSCH₂CH₂—, R₁₇=Phe side chain]: 6=2.91 (brs, 2H, CH₂), 3.84-3.18 (m, 2H, CH₂), 7.34 (brs, 5H, aryl CH of Phe), 8.33 (s, aryl CH).

Yield: 70-80%. ¹H NMR (TFA) [Rn=-Ala-Val-, R₁₇=Phe side chain]: δ=1.30 (m, 3H, CH₃), 1.52 (m, 3H, CH₃), 2.24 (m, 1H, CH), 2.38 (d, J=13.3 Hz, 2H, CH₂), 2.62-4.37 (m, 2H, CH₂) 4.8-5.59 (s, 1H, CH), 6.96-7.50 (brs, 5H, aryl CH of Phe), 8.74 (s, aryl CH).

Different St-Rn-PAspDET-co-PR₁₇ according of formula (10) were synthesized according to similar procedures. The introduction of ratios of the repeating unit were adjusted changing the mixing ratios of the corresponding monomer units to be allowed to react. Table 5 refers to amphiphilic copolymer St-Rn-PAspDET-co-R17 according to formula (10).

TABLE 5

| | | | Ratio^{a} | Mn^{b} | |
|---|---|---|---|---|---|
| Compound | Rn | R17 | PAsp:R17 | (KDa) | Ð |
| CP17 | CH₂CH₂SSCH₂CH₂— | Leu | 47:9 | 11.2 | 1.20 |
| CP18 | CH₂CH₂SSCH₂CH₂— | Leu | 54:4 | 12.0 | n.d |
| CP19 | CH₂CH₂SSCH₂CH₂— | Phe | 57:5 | 13.1 | 1.28 |
| CP20 | CH₂CH₂SSCH₂CH₂— | Phe | 37:9 | 9.6 | 1.24 |
| CP50 | -Ala-Val- | Phe | 87:10 | 19.9 | 1.16 |

^{a}Determined by NMR.
^{b}Determined by SEC.
Mn and DP refer to number average molar mass and degree of polymerization respectively.
Ð represent polydispersity as determined by SEC-MALS software analysis.

In the table above, R₁₇=Leu and R₁₇=Phe refer to a Leucine side chain or a Phenylalanine side chain respectively.

### Example 4. Synthesis of the Amphiphilic Block Copolymer with Formula (Ib4)

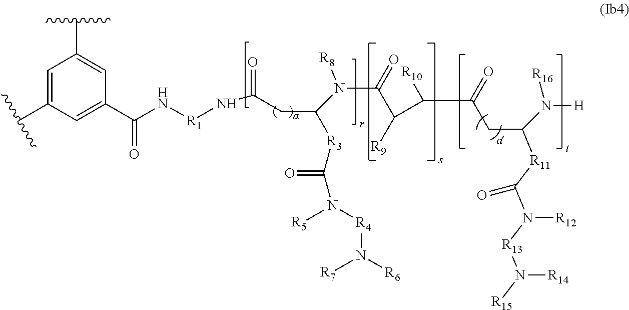

### Example 4A. Synthesis of Polycationic Polymers St-S—S-PAspDET/(DIIPA or Imidazolamine)-co-PR9 (11a)

In order to improve the efficiency of transfection in the polymeric system described in example 3, two different amines were introduced over the St-SS-PAsp(Bz)-co-PR9 (9) by an aminolysis reaction in the presence of DET, and DIIPA or 1-(3-aminopropyl)imidazole as second primary amine.

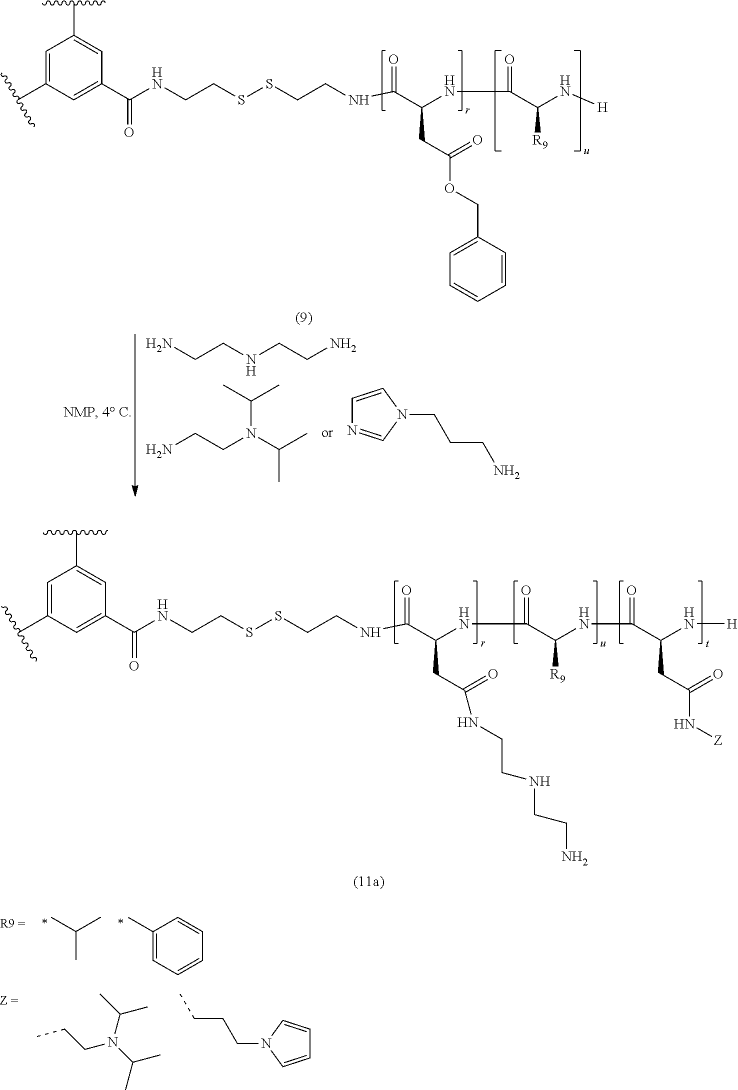

As described above (example 2A), the general procedure for the synthesis of the amphiphilic copolymer St-S—S-PAspDET/(DIIPA or Imidazolamine)-co-PR9 starts with the copolymer (DP of PBLA=45, 300 mg) being dissolved in NMP (10 mL) and cooled to 4° C. The resultant polymer solution was added dropwise to the mixture of DET (3 mL) and second primary amine. For DIIPA the ratio molar with respect to DET equivalents is 1:4, and for 1-(3aminopropyl)imidazole the ratio molar is 1:2. cooled at 4° C., and the mixture was stirred for 4 hours at this temperature. After this time, the reaction mixture was added dropwise into cold HCl 6M for neutralization (pH 3.5). The polymer product was purified by centrifugal-assisted ultrafiltration. After filtration, the remaining aqueous polymeric solution was lyophilized to obtain the final product.

Yield: 70-80%. ¹H NMR (D₂O) [R9=Leu side chain, Z=DIIPA]: δ=1.01-0.81 (m, 3H, CH3), 1.47-1.25 (m, 3H, CH3), 1.64 (brs, 1H, CH), 2.94 (m, 2H, CH2), 3.95-3.19 (m, 2H, CH2), 4.39 (brs, 1H, CH), 8.35 (s, aryl CH).

Yield: 70-80%. ¹H NMR (D₂O) [R9=Phe side chain], Z=DIIPA: δ=1.40 (m, 3H, CH₃), 2.93 (m, 2H, CH₂), 3.87-3.16 (m, 2H, CH₂), 7.38 (brs, 5H, aryl CH of Phe), 8.34 (s, aryl CH).

Yield: 70-80%. ¹H NMR (D₂O) [R9=Phe side chain, Z=aminopropylimidazole]: δ=2.15 (brs, 2H, CH₂), 2.56-3.12 (brs, 2H, CH₂), 3.14-3.87 (m, 2H, CH₂), 4.30 (brs, 2H, CH₂), 7.34 (brs, phenyl CH), 7.52 (s, Imidazole CH), 7.57 (s, Imidazole CH), 8.33 (s, aryl CH), 8.75 (s, Imidazole CH).

Different St-S—S-PAspDET/Z-co-PR9 according of formula (11a) were synthesized according to similar procedures. The introduction of ratios of the repeating unit were adjusted changing the mixing ratios of the corresponding monomer units to be allowed to react.

Table 6 refers to amphiphilic copolymers St-S—S-PAspDET/Z-co-PR9 according to formula (11a).

TABLE 6

| | | | | | | MW^{b} | |
|---|---|---|---|---|---|---|---|
| Compound | Z | R9 | DP_{Asp}_{^{a}} | DP_{DET/Z}_{^{a}} | DP_{R9}_{^{a}} | (Da) | Ð |
| CP21 | DIIPA | Leu | 66 | 48/18 | 4 | 15.2 | 1.06 |
| CP22 | DIIPA | Leu | 43 | 31/12 | 7 | 10.8 | 1.05 |
| CP23 | DIIPA | Phe | 51 | 41/10 | 5 | 12.5 | 1.03 |
| CP51 | aminopropylimidazole | Phe | 48 | 36/12 | 5 | 11.6 | 1.04 |

^{a}Ratio DET/Z and Asp/R9 determined by NMR.
^{b}Determined by SEC. MW and DP refer to number average molar mass and degree of polymerization respectively. Ð represent polydispersity as determined by SEC-MALS software analysis.

In the table above, R9=Leu and R9=Phe refer to a Leucine side chain or a Phenylalanine and Z refer to a DIIPA or aminopropylimidazole side chain respectively.

### Example 4B. Synthesis of Polycationic Polymers St-S—S-PAspDET-co-P(cyclic R9-R10) (11b)

In order to improve the efficiency of transfection in the polymeric system described in example 3, a new hydrophobic residue is introduced: polyproline. The synthetic strategy consists of using St-S—S-PAsp(Bz)(6) as initiator and polymerizing a terminal proline block. Once the diblock is generated, the aminolysis reaction is carried out with DET.

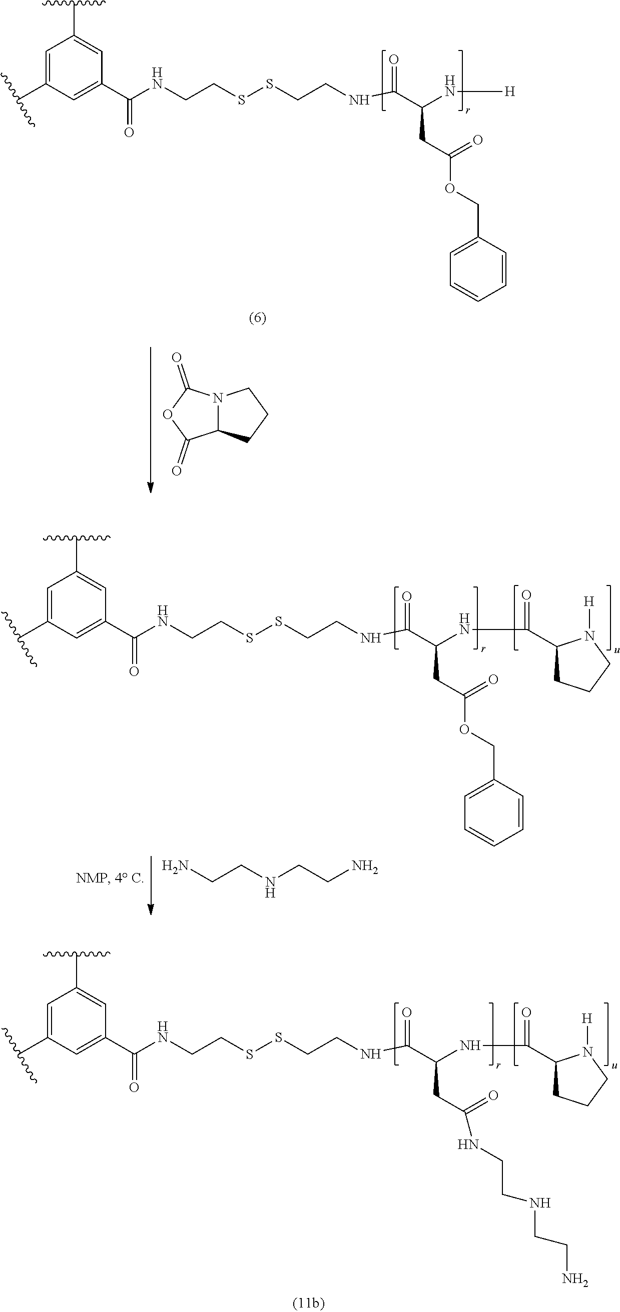

Different St-S—S-Pasp(Bz)-b-Ppro were synthesized according to similar procedures described above or below for similar products.

Table 7 shows the ratio of copolymer St-S—S-Pasp(Bz)-b-Ppro according to formula (11b).

TABLE 7

| Compound | R | DP_{Pasp(Bz)}^{theo} | DP_{R}^{theo} | DP_{Pasp(Bz)} ^{a} | DP_{R}^{a} |
|---|---|---|---|---|---|
| CP56 | Pro | 100 | 10 | 100 | 10 |
| CP57 | Pro | 100 | 20 | 110 | 14 |

^{a}Determined by NMR.

In the table above, R=Pro refers to a Proline side chain.

Yield: 65-80%. ¹H NMR (DMSO)]: δ=1.79 (brs, 2H, CH₂), 2.54-2.92 (m, 2H, CH₂), 3.56 (brs, H, CH), 4.62 (m, H, CH), 4.83-5.17 (m, 2H, benzyl CH₂), 7.10-7.45 (m, aryl CH), 7.82 (s, aryl CH).

As an example, herein we describe the synthetic method in which R9 and R10 in FIG. (Ib4) are combined together to form a proline ring moiety group. Copolymer of St-S-SPAsp(Bz)100-b-Ppro(10) (500 mg, DP:100) was dissolved in NMP (7 mL) and cooled to 4° C. The resultant copolymer solution was added dropwise to the mixture of DET (13 mL, 50 eq. vs unit of Pasp(Bz)) and the solution was stirred for 4 h at 4° C. under nitrogen atmosphere. After this time, the reaction mixture was added dropwise into cold HCl 6 M for neutralization (pH 3.5). The polymer product was purified by centrifugal-assisted ultrafiltration. After filtration, the remaining aqueous polymeric solution was lyophilized to obtain the final product.

Yield: 70-90%. ¹H NMR (D₂O) δ=1.78-2.52 (m, 2H, CH₂), 2.93 (brs, 2H, CH₂), 3.18-3.88 (m, 2H, CH₂), 8.36 (s, aryl CH).

Table 8 refers to amphiphilic copolymers St-S—S-PAspDET-b-Ppro according to formula (11b).

TABLE 8

| Compound | R | DP_{Asp}^{a} | DP_{Pro}^{a} | MW^{b} (Kda) | Ð |
|---|---|---|---|---|---|
| CP58 | Pro | 100 | 10 | 21.9 | 1.06 |
| CP59 | Pro | 119 | 14 | 26 | 1.20 |

^{a}determined by NMR.
^{b}Determined by SEC.
MW and DP refer to weight average molar mass and degree of polymerization respectively.
Ð represent polydispersity as determined by SEC-MALS software analysis.

### Example 5. Synthesis of Shielded Compounds with Formula (b5)

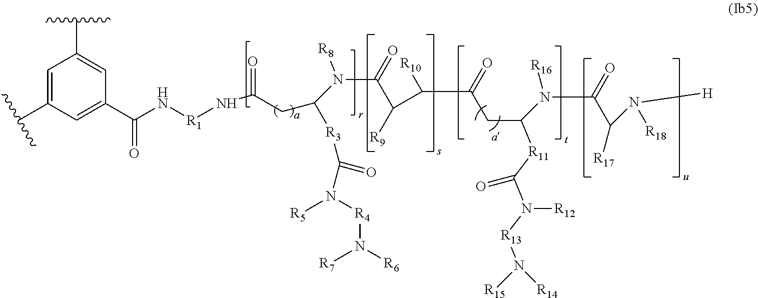

In order to improve the stability of the systems described along previous examples, a hydrophilic polymer such as polysarcosine was introduced. In this case, the use of sarcosine prevents from agglomeration and precipitation in complex media like blood, thus, enhancing their in vivo circulation times.

### Example 5A. Introduction of a Hydrophilic Fragment in the Synthesis of Block-Copolymers

We introduced polysarcosine as a hydrophilic block in all the polymers described in previous examples. Polymerization was carried out via ring opening polymerization mechanism, using St-S—S-Pasp(Bz) or St-S—S-Pasp(Bz)-co-R9 as the initiator.

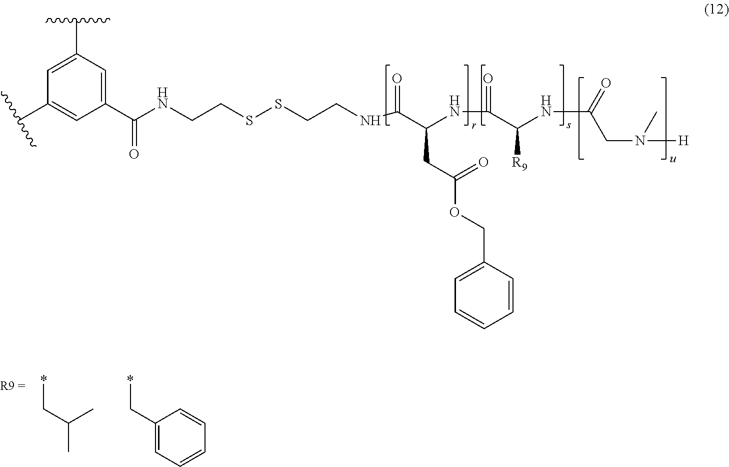

General Procedure for the Synthesis of St-S—S-Pasp(Bz)-b-Psar or St-S—S-Pasp(Bz)-co-PR9-b-Psar:

St-S—S-Pasp(Bz) or St-S—S-Pasp(Bz)-co-PR9 (0.083 mmol, DP=47) was added to a Schlenk tube fitted with a stirring bar and a stopper, then purged with 3 cycles of vacuum/N₂, and dissolved in anhydrous dichloromethane (22 mL). Then, the sarcosine-NCA (450 mg, 3.91 mmol, DP=47) was dissolved in DMF (2 mL) and was added to the reaction mixture. The mixture was stirred at 10° C. for 16 hours. Upon completion, the reaction mixture became clear and full conversion of the monomer could be detected by IR. The reaction mixture was poured into diethyl ether to precipitate the product. The precipitate was isolated by centrifugation (3750 rpm, 4 min) and dried under vacuum. Copolymer was isolated as a white solid.

Yield: 60-80%.

St-S—S-Pasp(Bz)-b-Psar: ¹H NMR (DMSO): δ=2.91-2.55 (m, 2H, CH₂), 4.45-3.81 (m, 2H, CH), 4.62 (brs, 1H, CH), 5.01 (brs, 2H, benzyl CH₂), 7.27 (brs, 5H, aryl CH), 8.16 (brs, 1H, amide), 8.41 (s, aryl CH).

St-S—S-Pasp(Bz)-co-Pphe-b-Psar: ¹H NMR (TFA): δ=3.40-2.92 (m, 2H, CH₂), 4.75-4.41 (m, 2H, CH), 5.03 (brs, 1H, CH), 5.23 (brs, 2H, benzyl CH₂), 7.32 (brs, 5H, aryl CH), 8.61 (s, 1H). St-S—S-Pasp(Bz)-co-Pleu-b-Psar: ¹H NMR (TFA): δ=0.84 (m, 3H, CH₃), 1.58 (m, 1H), 3.23-2.85 (m, 2H, CH₂), 4.57-4.18 (m, 2H, CH), 4.90 (brs, 1H, CH), 5.11 (brs, 2H, benzyl CH₂), 7.22 (brs, 5H, aryl CH), 8.44 (s, 1H).

Different St-S—S-Pasp(Bz)-co-PR9-b-Psar according of formula (12) were synthesized according to similar procedures. The introduction of ratios of the repeating unit were adjusted changing the mixing ratios of the corresponding monomer units to be allowed to react.

Table 9 refers to amphiphilic copolymer St-S—S-Pasp(Bz)-co-PR9-b-Psar according to formula (12).

TABLE 9

| Compound | R9 | DP_{Asp}^{a} | DP_{Psar}^{a} | DP_{R9}^{a} |
|---|---|---|---|---|
| CP24 | * | 42 | 40 | — |
| CP25 | * | 42 | 20 | — |
| CP26 | Leu | 68 | 71 | 6 |
| CP27 | Leu | 59 | 70 | 11 |
| CP28 | Phe | 45 | 66 | 5 |
| CP29 | Phe | 40 | 53 | 10 |

^{a}DP determined by ¹H-NMR

Compounds CP24 and CP25, corresponds to compounds according to (12) wherein s=0. R9=Leu and R9=Phe refers to the Leucine side chain and Phenylalanine side chain respectively.

### Example 5B. Synthesis of Polyaspartamide Derivatives (St-S—S-PAspDET-Co-PR9-b-Psar)

The aminolysis reaction was carried out for the different block polymers synthesized in example 5A.

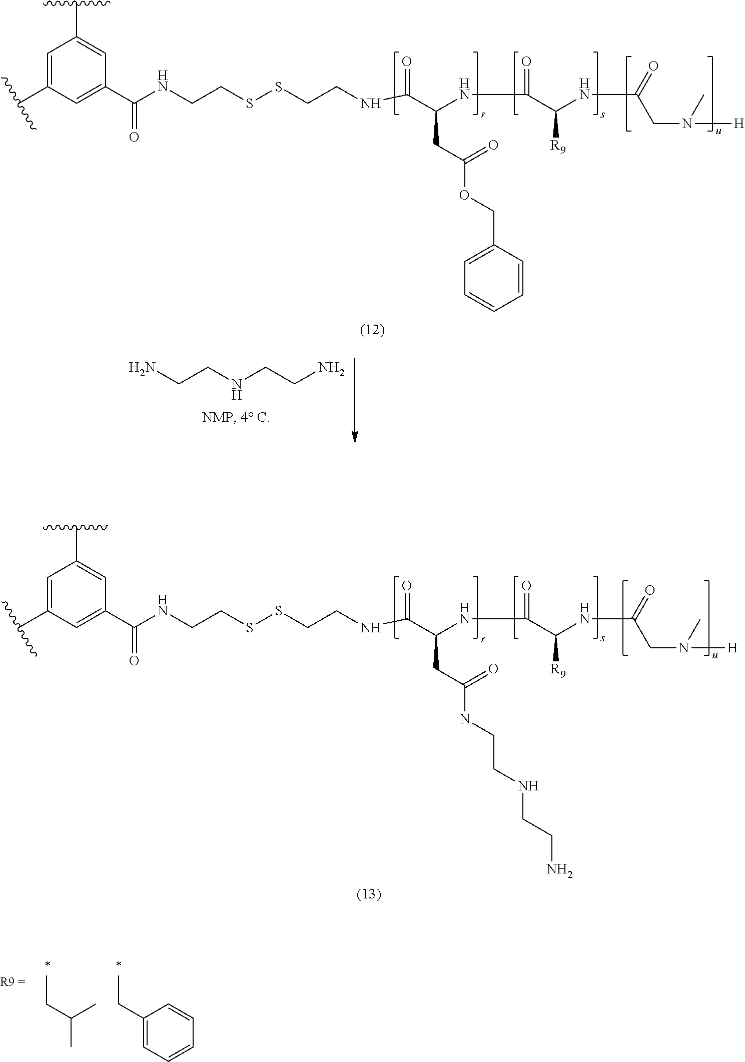

### General Procedure for Aminolysis of St-S—S-PAspDET-Co-PR9-b-Psar (13):

As an example, herein we describe the synthetic method in which R9 can hold either anhydrophobic polymer such as polyleucine or polyphenylalanine, or the polyaspartic polymer.

The polymer (200 mg of PBLA) was dissolved in NMP (4 mL) and cooled to 4° C. The resultant copolymer solution was added dropwise to the mixture of DET (5.3 mL, 50 eq. vs unit of Pasp(Bz)) and the solution was stirred for 4 h at 4° C. under nitrogen atmosphere. After this time, the reaction mixture was added dropwise into cold HCl 6 M for neutralization (pH 3.5). The polymer product was purified by centrifugal-assisted ultrafiltration. After filtration, the remaining aqueous polymeric solution was lyophilized to obtain the final product.

Yield: 60-80%. St-S—S-PAspDET-b-Psar ¹H NMR (D₂O): δ=3.18-2.60 (m, 2H, CH₂ Pasp+3H, CH₃ Psar), 3.65-3.25 (m, 2H, CH₂), 4.40 (m, 2H, CH₂), 8.33 (s, 1H).

Yield: 60-80%. St-S—S-PAspDET-co-Pleu-b-Psar ¹H NMR (D₂O): δ=0.94 (d, J=14.7 Hz, 3H, CH₃), 1.65 (brs, 1H, CH), 3.19-2.69 (m, 2H, CH₂ Pasp+3H, CH₃ Psar), 3.73-3.23 (m, 2H, CH₂), 4.64-4.10 (m, 2H, CH₂), 8.34 (s, 1H).

Yield: 60-80%. St-S—S-PAspDET-co-Pphe-b-Psar ¹H NMR (D₂O): δ=3.17-2.67 (m, 3H, CH₃ Psar) 3.72-3.19 (m, 2H, CH₂), 4.40 (m, 2H, CH₂), 7.36 (brs, 5H, CH aryl), 8.33 (s, 1H).

Different St-S—S-Pasp(DET)-co-PR9-b-Psar according of formula (13) were synthesized according to similar procedures. The introduction of ratios of the repeating unit were adjusted changing the mixing ratios of the corresponding monomer units to be allowed to react.

Table 10 refers to amphiphilic copolymer St-S—S-PAspDET-co-PR9-b-Psar according to formula (13)

TABLE 10

| | | | | | MW^{b} | |
|---|---|---|---|---|---|---|
| Compound | R9 | DP_{PAspDET}_{^{a}} | DP_{Psar}_{^{a}} | DP_{R9}_{^{a}} | (Kda) | Ð |
| CP30 | * | 42 | 37 | 0 | 11.8 | 1.12 |
| CP31 | * | 41 | 22 | 0 | 10.6 | 1.09 |
| CP32 | Leu | 46 | 50 | 8 | 14.5 | 1.03 |
| CP33 | Phe | 51 | 44 | 5 | 14.8 | 1.07 |
| CP34 | Phe | 48 | 36 | 9 | 14.5 | 1.05 |

^{a}Ratio PAspDET/R9/Psar determined by NMR.
^{b}Determined by SEC. Mw and DP refer to number average molar mass and degree of polymerization respectively. Ð represent polydispersity as determined by SEC-MALS software analysis.

*Compounds CP30 and CP31, corresponds to compounds according to (13) wherein s=0. R9=Leu and R9=Phe refers to the Leucine side chain and Phenylalanine side chain respectively.

### Example 5C. Synthesis of Polyaspartamide Derivatives (St-S—S-PAspDET/DIIPA-Co-PR9-b-Psar) (14)

In order to improve the efficiency of transfection in the polymeric system, as described above in example 4, two types of amines were introduced over the amphiphilic systems by an aminolysis reaction in the presence of DET and DIIPA.

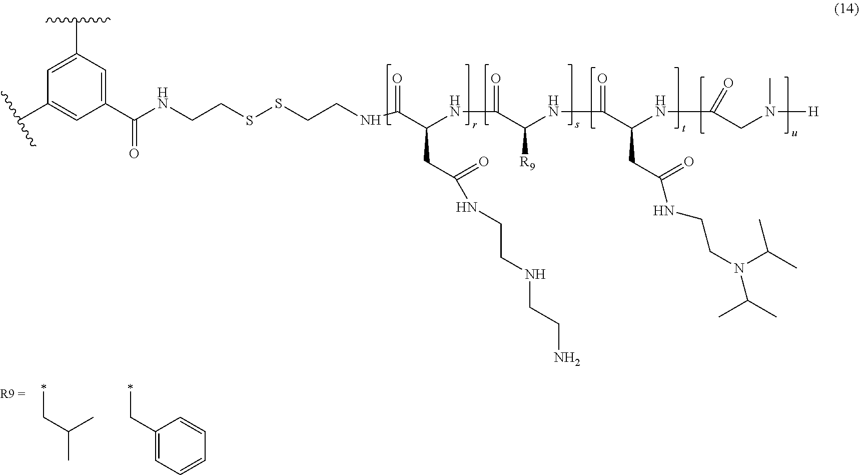

The strategy carried out is similar to that described above for the aminolysis product in example 4, point 4A.

Yield: 60-80%. St-S—S-PAspDET/DIIPA-b-Psar ¹H NMR (D₂O): δ=1.41 (d, J=6.4 Hz, 3H, CH₃), 3.19-2.73 (m, 3H, CH₃), 3.68-3.23 (m, 2H, CH₂), 3.93-3.73 (m, 2H, CH₂), 4.61-4.08 (m, 2H, CH₂), 8.36 (s, 1H).

Yield: 60-80%. St-S—S-PAspDET/DIIPA-co-Pleu-b-Psar ¹H NMR (D₂O): δ=0.95 (brs, 3H, CH₃), 1.40 (s, 3H, CH₃), 3.02-2.76 (m, 3H, CH₃), 3.57-3.04 (m, 2H, CH₂), 3.78 (m, 2H, CH₂), 4.57-4.10 (m, 2H, CH₂), 8.35 (s, 1H).

Yield: 60-80%. St-S—S-PAspDET/DIIPA-co-Pphe-b-Psar ¹H NMR (D₂O): δ=1.40 (brs, 3H, CH₃), 2.94 (m, 3H, CH₃), 3.90-3.18 (m, 2H, CH₂), 4.40 (m, 2H, CH₂), 7.34 (brs, 5H, CH aryl), 8.35 (s, 1H).

Different St-S—S-PAspDET/DIIPA-co-PR9-b-Psar according of formula (14) were synthesized according to similar procedures. The introduction of ratios of the repeating unit were adjusted changing the mixing ratios of the corresponding monomer units to be allowed to react.

Table 11 refers to amphiphilic copolymer St-S—S-PAspDET/DIIPA-co-PR9-b-Psar according to formula (14)

TABLE 11

| | | | | | | MW^{b} | |
|---|---|---|---|---|---|---|---|
| Compound | R9 | DP_{PAspDET}_{^{a}} | DP_{PAspDIIPA}_{^{a}} | DP_{Psar}_{^{a}} | DP_{R9}_{^{a}} | (Kda) | Ð |
| CP35 | * | 51 | 20 | 58 | 0 | 20.3 | 1.08 |
| CP36 | Leu | 38 | 21 | 50 | 11 | 19.9 | 1.02 |
| CP37 | Phe | 46 | 13 | 28 | 11 | 17.0 | 1.06 |

^{a}Ratio PAspDET/DIIPA/PR9/Psar determined by NMR.
^{b}Determined by SEC. MW and DP refer to number average molar mass and degree of polymerization respectively. Ð represent polydispersity as determined by SEC-MALS software analysis.

*Compound CP35 correspond to the compound according to (14) wherein s=0. R9=Leu and R9=Phe refers to the Leucine side chain and Phenylalanine side chain respectively.

### Example 6. Synthesis of Shielded Compounds with Formula (b6)

As an alternative to the systems described in example 5, a hydrophilic block was introduce to the side chains of DET by graft into.

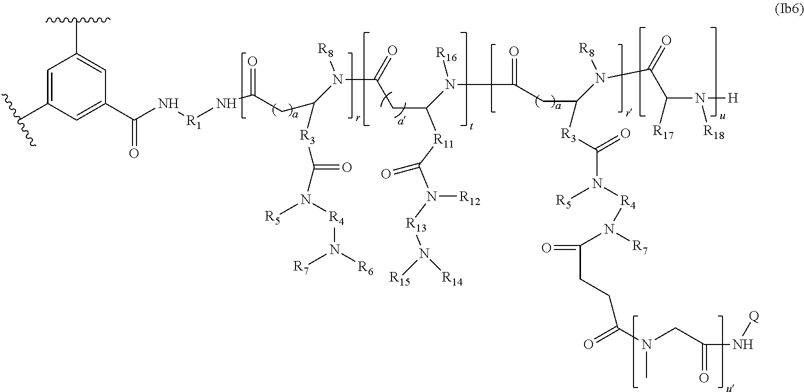

### Example 6A. Synthesis of Shielded Polyaspartamide Derivatives (St-S—S-PAspDET/DIIPA-graft into-Succ-Psar-Q) (15)

Following this route, a variety of shielded polyaspartamide derivatives were synthesized.

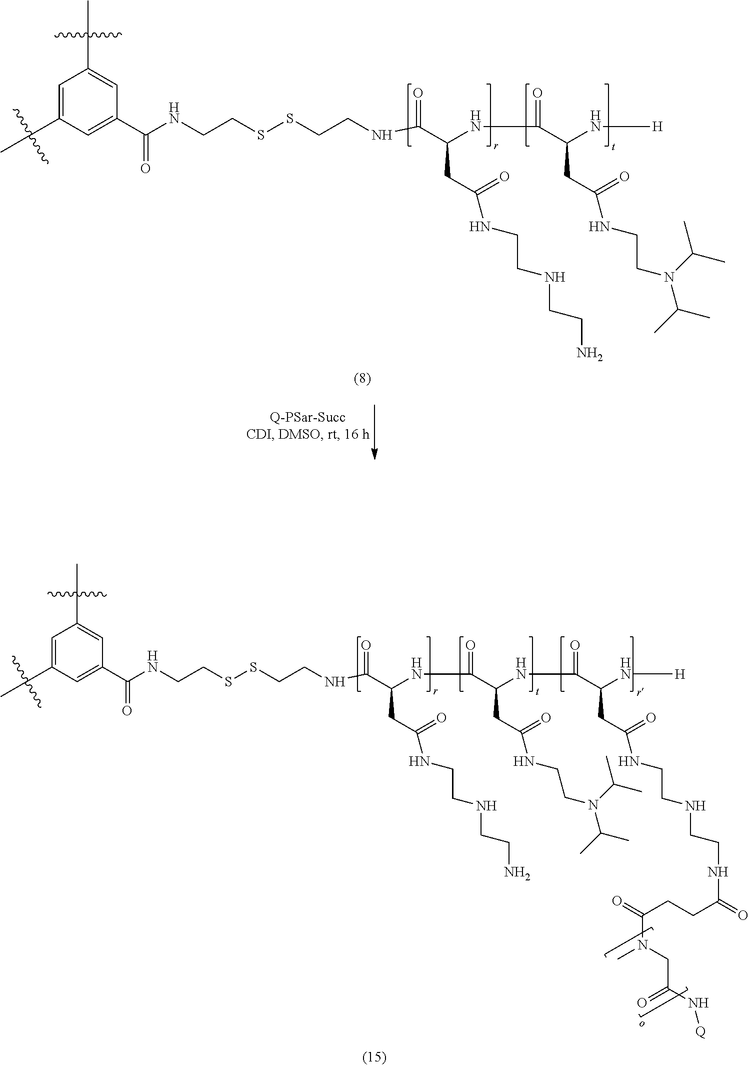

General Procedure for Peptide Coupling with 1,1′-Carbonyldiimidazole Coupling Reagent (CDI):

Q-PSar-Succ (0.029 mmol, 6% conjugation) was added to a two-necked round bottom flask fitted with a stirring bar and a stopper, then purged with 3 cycles of vacuum/N₂, and dissolved in 2 mL of DMSO. Then, CDI (3 eq, 0.089 mmol, 15 mg) was added to the reaction mixture and stirred for 30 minutes at room temperature. After this time, the polymer (St-S—S-PAspDET: 100 mg, 0.499 mmol) dissolved in 2 mL of DMSO was added. The mixture was stirred at room temperature for 16 hours. The reaction mixture was poured into THE to precipitate the product. Precipitate was isolated by centrifugation (3750 rpm, 4 min) and dried under vacuum. The final product was isolated as a white solid.

Yield: 60-80%. St-S—S-PAspDET-graft into-Succ-PSar ¹H NMR (D₂O): δ=3.18-2.60 (m, CH₂+CH₃), 3.65-3.25 (m, 2H, CH₂), 4.40 (m, 2H, CH₂), 8.33 (s, 1H)

Different St-S—S-PAspDET/DIIPA-graft into-Succ-PSar-Q according of formula (15), were synthesized according to similar procedures. The introduction of ratios of the repeating unit were adjusted changing the mixing ratios of the corresponding monomer units to be allowed to react.

Table 12 refers to different St-S—S-PAspDET-graft into-Succ-PSar-Q according of formula (15).

TABLE 12

| | | | | | | MW^{b} |
|---|---|---|---|---|---|---|
| Compound | DP_{PAspDET} | DP_{PAspDIIPA} | Q | % conjug | DP_{PSar}_{^{a}} | (KDa) |
| CP38 | 100 | * | Galactosamine | 3 | 142 | 40.7 |
| CP39 | 91 | 29 | n-Bu | 6 | 138 | 36.4 |

^{a}Ratio PAspDET/PSar determined by NMR.
^{b}Determined by SEC.
*Compound CP38 correspond to the compound according to (15) wherein t = 0. % Conjug stands for the number of Poly-Sarcosine chains conjugated vs number of PAspartic-DET monomeric units.

### Example 6B. Synthesis of Shielded Detachable Polyaspartamide Derivatives (St-S—S-PAspDET-Co-PPhe-graft into-Succ-detachable-PSar-Q) (16)

Design of a new family of structures with detachable shielding which improves transfection without compromising the stability of the polymer in an aqueous medium:

### Synthesis of Shielded Detachable Based on Bioreducible Polymers:

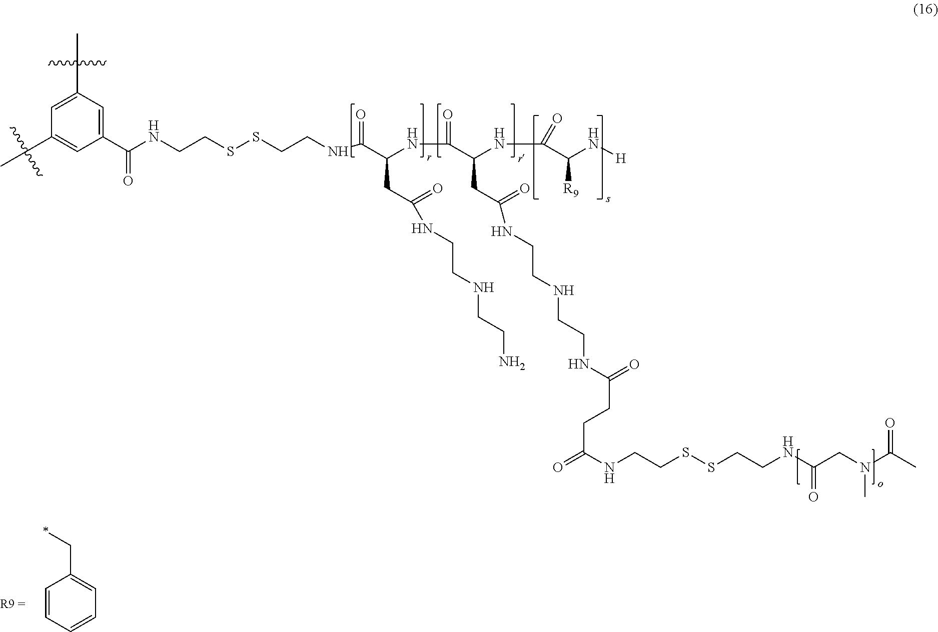

To generate this type of structures, a polysarcosine detachable with an S—S bond was synthesized following this synthetic route:

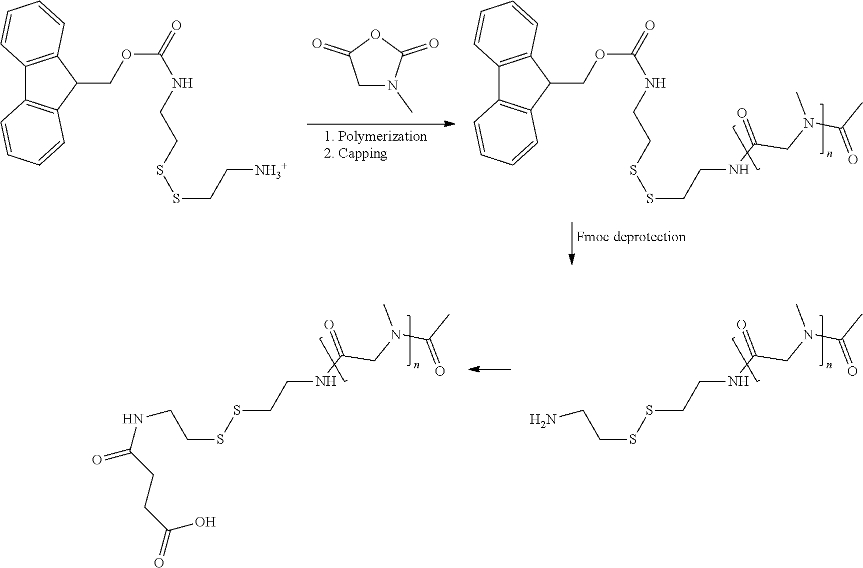

### General Procedure for the Polymerization of PSar-COCH₃:

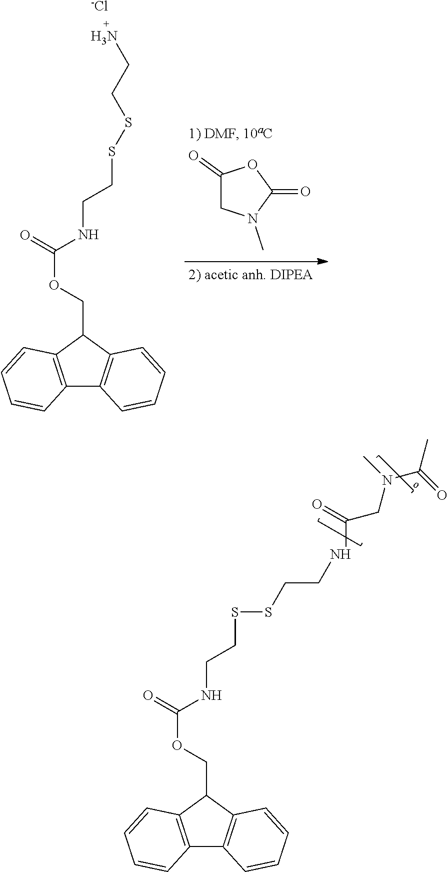

Sarcosine-NCA (1675 mg, 14.59 mmol) was added to a Schlenk tube fitted with a stirring bar, a stopper, then purged with 3 cycles of vacuum/N₂, and dissolved in 5 mL of anhydrous DMF. Then, the 2-((2-((((9H-fluoren-9-yl)methoxy)carbonyl)amino)ethyl)disulfanyl)ethanaminium chloride was dissolved in DMF (2 mL) and added to the reaction mixture. The mixture was stirred at 10° C. for 16 hours. Upon completion, the reaction mixture became clear. Full conversion of the monomer could be detected by IR.

Then, DIPEA (1 eq, 84.20 mL) and acetic anhydride (10 eq, 460 mL) were added, and the mixture was stirred at the same temperature for 1 hour. After this time, the reaction mixture was poured into diethyl ether to precipitate the product. The precipitate was isolated by centrifugation (3750 rpm, 4 min) and dried under vacuum. The final product was isolated as a white solid.

Yield: 65%. ¹H NMR (TFA): δ=2.71 (s, 3H, CH₃), 2.85 (dd, J=11.8; 5.2 Hz, 2H), 3.16-2.97 (m, 2H, CH₂), 3.77-3.661 (m, 2H, CH₂), 3.97-3.87 (m, 2H, CH₂), 4.63-4.18 (m, 2H, CH₂).

### General Procedure for Fmoc Deprotection:

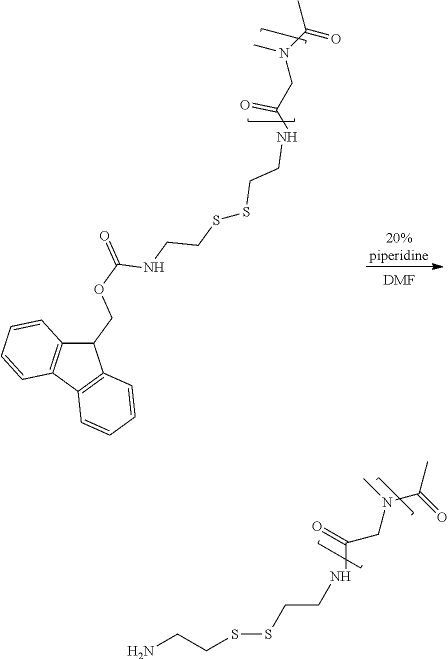

Polysarcosine (750 mg) was added to a round bottom flask fitted with a stirring bar, a stopper and purged with 3 cycles of vacuum/N₂, then dissolved in a mixture of 5 mL of anhydrous DMF and 1 mL of piperidine. The mixture was stirred at room temperature for 2 hours. After this time, the reaction mixture was poured into diethyl ether to precipitate the product. The precipitate was isolated by centrifugation (3750 rpm, 4 min) and dried under vacuum. The final product was isolated as a white solid.

Yield: 90%. ¹H NMR (D₂O): δ=2.01 (d, J=9.7 Hz, 4H, CH₂), 2.22 (s, 3H, CH₃), 3.17-2.70 (m, 3H, CH₃), 4.63-4.01 (m, 2H, CH₂), 7.26 (m, 1H, CH aryl), 7.53 (s, 1H, aryl CH).

### General Procedure for the Synthesis of Succ-Ethyl-S—S-PSar-COCH₃:

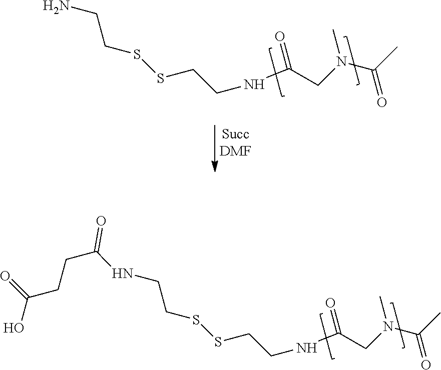

Polysarcosine (400 mg) was added to a round bottom flask fitted with a stirrer, a stopper and purged with 3 cycles of vacuum/N2, then dissolved in a 3 of DMF. Succinate anhydride (10 eq, 1.34 mmol, 144 mg) was added and the mixture was stirred at room temperature for 16 hours. After this time, the reaction mixture was poured into diethyl ether to precipitate the product. The precipitate was isolated by centrifugation (3750 rpm, 4 min) and dried under vacuum. The final product was isolated as a white solid.

Yield: 75% ¹H NMR (D₂O): δ=2.04 (d, J=9.2 Hz, 2H, CH₂), 2.19 (s, 3H, CH₃), 2.65 (m, 2H, CH₂), 3.27-2.81 (m, 3H, CH₂), 3.58 (m, 2H, CH₂), 4.32 (m, 2H CH₂).

Once the detachable polysarcosine was synthesized, the coupling reaction was carried out according to the procedure disclosed above for example 6 over all polymeric systems PAspDET included in this document. This methodology generates new detachable polycationic systems according to formula (16) as above.

Different St-S—S-PAspDET-PR9/graft into-[Succ-ethyl-S—S-ethyl]-PSar according of formula (16) were synthesized according to similar procedures. The introduction of ratios of the repeating unit were adjusted changing the mixing ratios of the corresponding monomer units to be allowed to react.

Table 13 refers to different St-S—S-PAspDET-PR9/graft into-[Succ-ethyl-S—S-ethyl]-PSar according of formula (16)

TABLE 13

| | | | | | | MW^{b} |
|---|---|---|---|---|---|---|
| Compound | R9 | % conj. | DP_{PAspDET}_{^{a}} | DP_{PSar}_{^{a}} | DP_{R9}_{^{a}} | (Da) |
| CP40 | — | 3 | 100 | 84 | 0 | 28.3 |
| CP41 | — | 2 | 100 | 56 | 0 | 25.8 |
| CP42 | — | 1 | 100 | 28 | 0 | 23.3 |
| CP43 | — | 6 | 100 | 168 | 0 | 35.6 |
| CP44 | Phe | 3 | 57 | 84 | 5 | 19.1 |

^{a}Ratio PAspDET/R9/PSar determined by ¹H-NMR.
^{b}Determined by SEC. MW and DP refer to number average molar mass and degree of polymerization, respectively.
*Compounds CP40, CP41, CP42 and CP43 correspond to the compounds according to (16) wherein s = 0. R9 = Phe refers to the Phenylalanine side chain. % Conjug stands for the number of Poly-Sarcosine chains conjugated vs number of PAspartic-DET monomeric units.

### Example 7. Synthesis of Biotagged Compounds with Formula (Ib7)

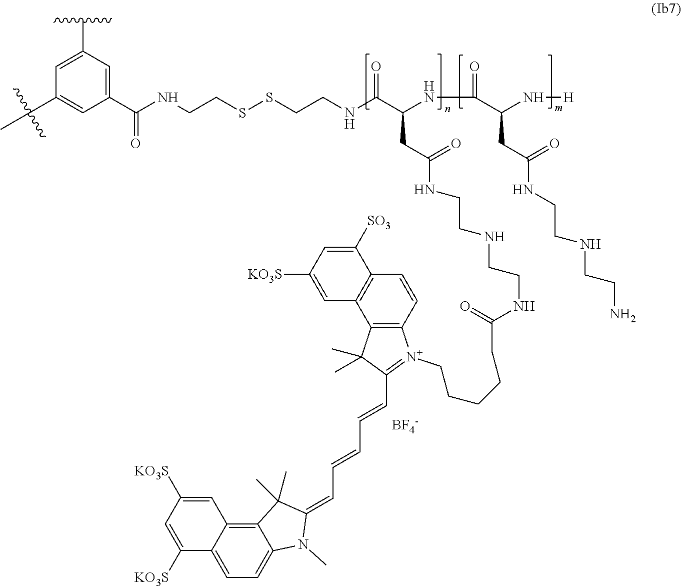

The cationic polymers described in previous examples have been biotagged with sulfo cy5.5 in order to quantify the efficiency of transfection and their biodistribution in the organism.

### General Procedure for Biotagged St-S—S-PAspDET:

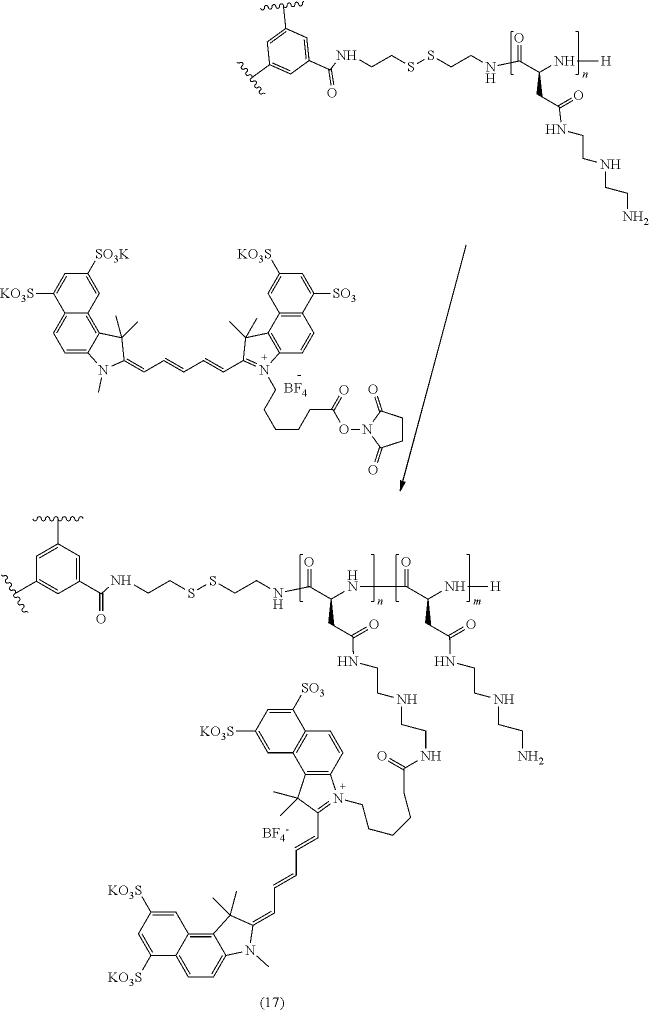

St-S—S-PAspDET(100) (100 mg, 0.36 mmol) was added to a two-necked round bottom flask fitted with a stirring bar and a stopper, then purged with 3 cycles of vacuum/N2, and dissolved in 3 mL of DMSO. Then, Sulfo cy5.5 (0.1 eq, 2.98 mg, 0.0036 mmol) was dissolved in 1 mL of DMSO and added to the reaction mixture and stirred for 16 hours at room temperature. The reaction mixture was poured into THE to precipitate the product. The precipitate was isolated by centrifugation (3750 rpm, 4 min) and dried under vacuum. The final product was isolated as a white solid. (q=95%)

The load of sulfo cy5.5 in the final product was determined by fluorescence.

### Example 7A. Synthesis of Shielded Biotagged Polyaspartamide Derivatives (St-S—S-PAspDET-Sulfo cy5.5-graft into-Succ-PSar-Galactosamine)

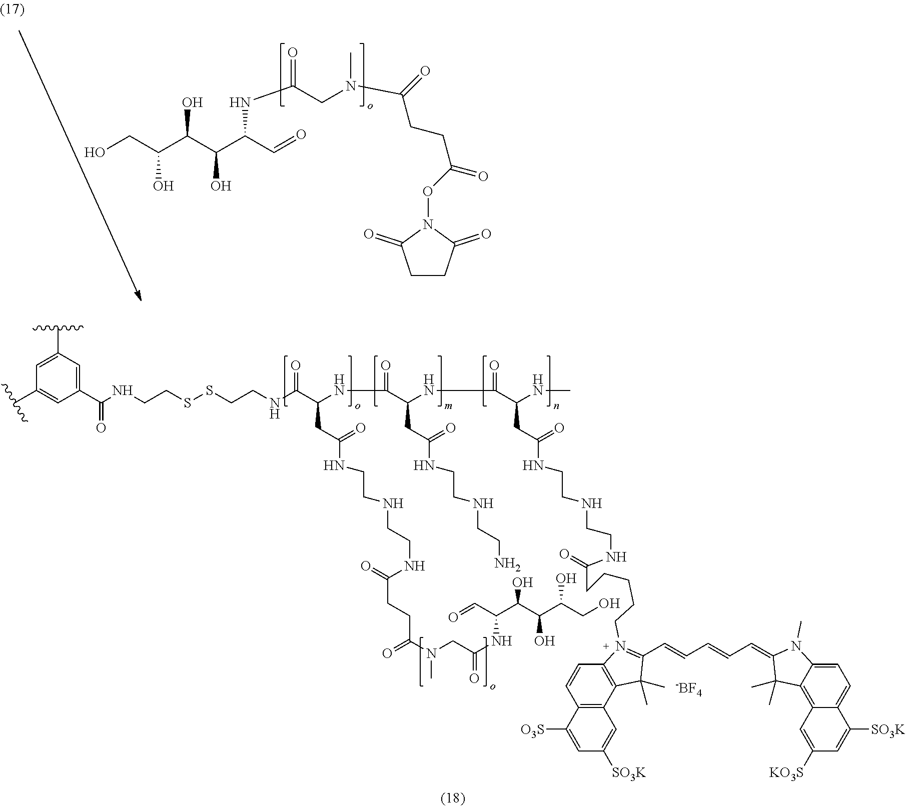

The hydrophilic fragment is introduced to the biotagged compound by coupling graft into as described in example 6.

Yield: 60-80%. St-S—S-PAspDET-sulfo cy^{5.5}-graft into-Succ-PSar-Galactosamine ¹H NMR (D₂O): δ=3.18-2.60 (m, CH₂+CH₃), 3.65-3.25 (m, 2H, CH₂), 4.40 (m, 2H, CH₂), 8.33 (s, 1H)

Table 14 refers to St-S—S-PAspDET-sulfo cy^{5.5}-graft into-Succ-PSar-Galactosamine according of formula (18)

TABLE 14

| | | | | | MW^{b} |
|---|---|---|---|---|---|
| Compound | DP_{PAspDET} | Q | % conjug | DP_{PSar}^{a} | (KDa) |
| CP45 | 100 | Galactosamine | 3 | 142 | 41.9 |

^{a}DP PAspDET and DP PSar determined by ¹H-NMR.
^{b}Calculated from SEC of precursor (CP38). MW and DP refer to number average molar mass and degree of polymerization, respectively. % Conjug stands for the number of Poly-Sarcosine chains conjugated vs number of PAspartic-DET monomeric units.

### Example 8. Synthesis of Lipidic Polyaspartamide Derivatives (St-S—S-PAspDET-Graft into-Lipoic Acid) (19)

Following this route, a lipidic polyaspartamide derivatives was synthesized.

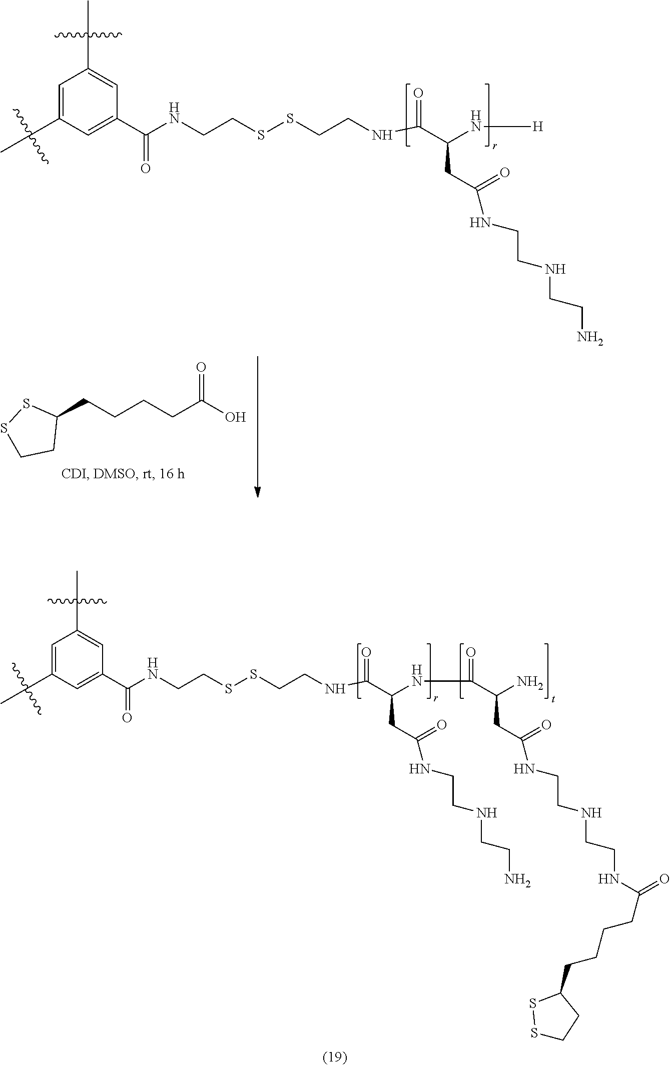

General Procedure for Peptide Coupling with 1,1′-Carbonyldiimidazole Coupling Reagent (CDI):

Lipoic acid (0.02 mmol, 5% conjugation) was added to a two-necked round bottom flask fitted with a stirring bar and a stopper, then purged with 3 cycles of vacuum/N₂, and dissolved in 2 mL of DMSO. Then, CDI (3 eq, 0.06 mmol, 10 mg) was added to the reaction mixture and stirred for 30 minutes at room temperature. After this time, the polymer (St-S—S-PAspDET: 80 mg, 0.4 mmol) dissolved in 1 mL of DMSO was added. The mixture was stirred at room temperature for 16 hours. The reaction mixture was poured into THE to precipitate the product. Precipitate was isolated by centrifugation (3750 rpm, 4 min) and dried under vacuum. The final product was isolated as a white solid.

Yield: 60-80%. St-S—S-PAspDET(72)-graft into-lipoic acid (5) ¹H NMR (D₂O): δ=1.34-1.56 (m, 2H, CH₂), 1.61-1.84 (m, 2H, CH₂), 2.04 (td, J=6.6, 13.3 Hz, 1H, CH), 2.35 (t, J=7.1 Hz, 2H, CH₂), 2.53 (dt, J=6.1, 18.6 Hz, 2H, CH₂), 2.89 (brs, 2H, CH₂), 3.02-3.81 (m, 2H, CH₂), 8.36 (s, aryl CH).

Table 15 refers to St-S—S-PAspDET(72)-graft into-lipoic acid(5) of formula (19)

TABLE 15

| Compound | DP_{PAspDET}^{a} | Conj units Lipoic acid^{b} | MW^{c} (KDa) |
|---|---|---|---|
| CP52 | 72 | 5 | 17.4 |

^{a}DP PAspDET determined by SEC of precursor.
^{b}Determined by ¹H NMR
^{c}Calculated by combining precursor MW by SEC and conjugated units by ¹H NMR. MW and DP refer to number average molar mass and degree of polymerization, respectively. Conj. units stands for the number of lipoic acid conjugated.

### Example 9. Synthesis of Lipidic Polyaspartamide Derivatives (St-S—S-PAspDET/Imidazolamine-Graft into-Lipidic Chain)

Following this route, a variety of lipidic polyaspartamide derivatives were synthesized.

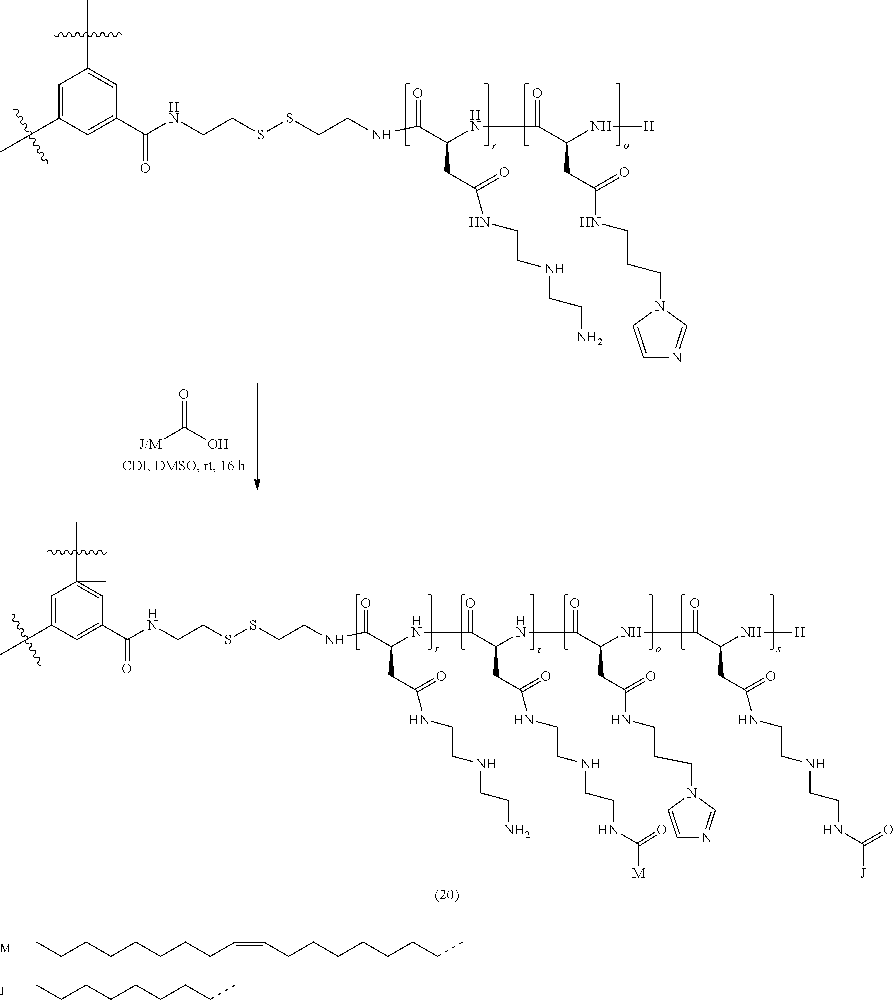

General Procedure for Peptide Coupling with 1,1′-Carbonyldiimidazole Coupling Reagent (CDI):

The experimental procedure for the synthesis of the polymer St-S—S-PAspDET/Imidazolamine-graft into-lipidic chain consists of a peptide coupling with CDI. The procedure is the same as described in example 7, using in this case other lipidic acids such as oleic and nonanoic acid.

Yield: 60-80%. St-S—S-PAspDET(65)/Imidazolamine(26)-graft into-oleic acid(5) 1H NMR (D₂O): δ=0.89 (brs, 3H, CH₃), 1.29 (s, 2H, CH₂), 1.60 (s, 2H, CH₂), 2.03 (s, 2H, CH₂), 2.15 (s, 2H, CH₂), 2.30 (s, 2H, CH₂), 2.50-3.12 (brs, 2H, CH₂), 3.14-3.75 (m, 2H, CH₂), 4.29 (s, 2H, CH₂), 5.35 (m, CH), 7.51 (s, Imidazole CH), 7.56 (s, 1H, Imidazole CH); 8.36 (s, aryl CH), 8.73 (s, 1H, Imidazole CH).

Yield: 60-80%. St-S—S-PAspDET(65)/Imidazolamine(26)-graft into-oleic acid(5)/nonanoic acid(13)¹H NMR (D₂O): δ=0.89 (brs, 3H, CH₃), 1.29 (s, 2H, CH₂), 1.60 (s, 2H, CH₂), 2.03 (s, 2H, CH₂), 2.15 (s, 2H, CH₂), 2.30 (s, 2H, CH₂), 2.50-3.12 (brs, 2H, CH₂), 3.14-3.75 (m, 2H, CH₂), 4.29 (s, 2H, CH₂), 5.35 (m, CH), 7.51 (s, Imidazole CH), 7.56 (s, 1H, Imidazole CH); 8.36 (s, aryl CH), 8.73 (s, 1H, Imidazole CH).

Table 16 refers to different St-S—S-PAspDET/Imidazoleamine-graft into-lipidic chain according of formula (20).

TABLE 16

| | | | | | Units | Units | MW^{c} |
|---|---|---|---|---|---|---|---|
| Compound | DP_{PAspDET}_{^{a}} | DP_{PAsplmizadolamine}_{^{a}} | M | J | of M^{b} | of J^{b} | (KDa) |
| CP53 | 65 | 26 | Oleic | | 5 | | 17.4 |
| | | | acid | | | | |
| CP54 | 65 | 26 | Oleic | Nonaic | 5 | 13 | 23.9 |
| | | | acid | acid | | | |

^{a}DP PAspDET calculated by combination of SEC of precursor and 1H NMR.
^{b}Calculated by ¹H NMR.
^{c}Calculated by combination of SEC of precursor and 1H NMR. MW and DP refer to weight average molar mass and degree of polymerization, respectively.

### Example 10: Synthesis of 6 Arm St-Lys(3)-polyaspartamide Derivatives

General procedure for the polymerization of 6 arm St-Lys(3)-PAsp(OBzl) is the same as described in example 1B.

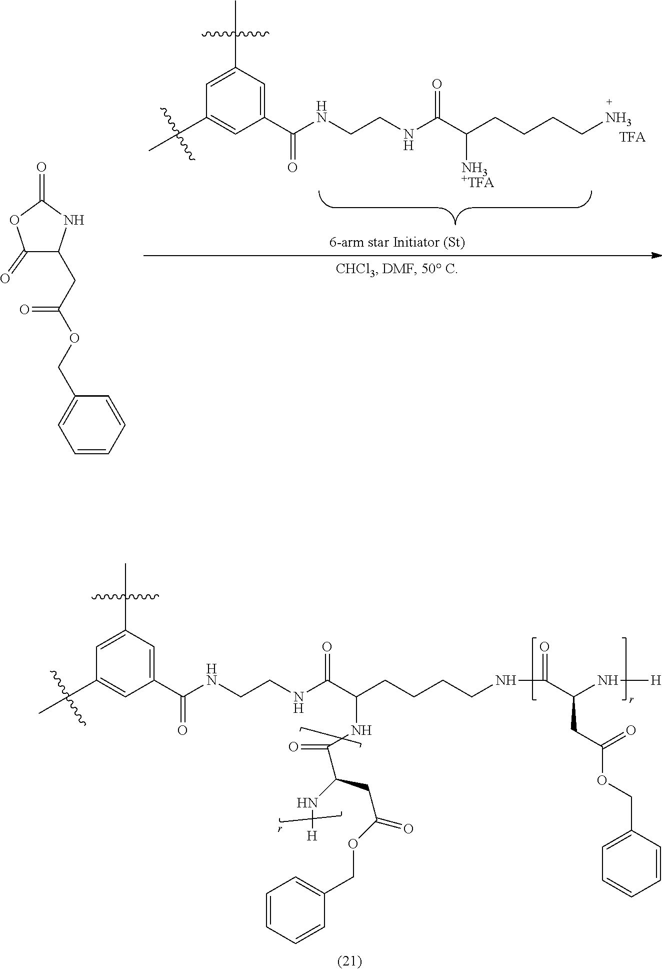

Yield: 70-90%¹H NMR[St-Lys(3)-PAspDET(100)](TFA): δ=1.51 (m, 3H, CH₃), 1.84-1.95 (m, 2H, CH₂), 2.80-3.26 (m, 2H, CH₂), 3.48 (m, 2H, CH₂), 4.97 (brs, 1H, CH), 5.15 (m, 2H, benzyl CH₂), 7.34 (brs, 5H, aryl CH), 8.68 (s, aryl CH).

Aminolysis Reaction of poly(β-benzyl-L-aspartate) (21) to afford St-Lys(3)-PAsp-oligoamine (22)

### General Procedure for the Aminolysis Reaction to Generate Polycationic Homopolymer

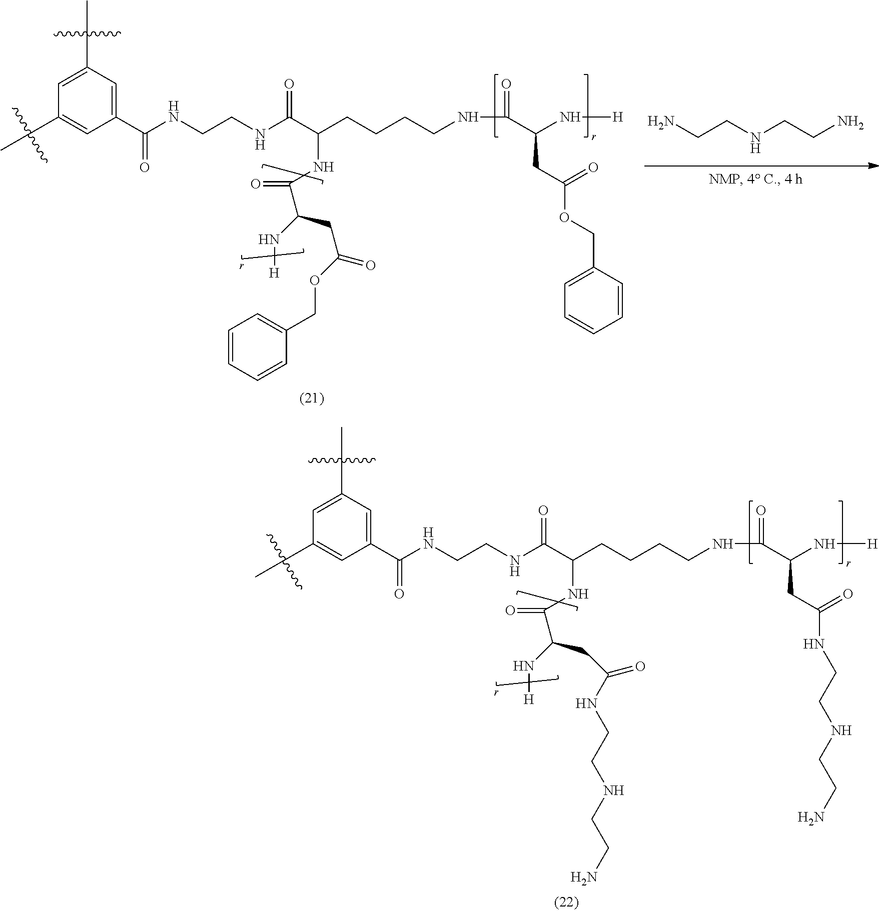

St-[Lys(3)-PAsp(Bz)(21) (DP=100, 750 mg) was dissolved in NMP (15 mL) and cooled to 4° C. This solution was added dropwise to the oligoamine (50 eq DET, vs unit of Asp) cooled at 4° C. and the mixture was stirred for 4 hours at the same temperature. After this time, the reaction mixture was added dropwise into cold HCl 6M for neutralization (pH 3.5). The polymer product was purified by centrifugal-assisted ultrafiltration. After filtration through 0.22 um PES filter, the remaining aqueous polymeric solution was lyophilized to obtain the final product (400 mg, q=60%).

¹H NMR [St-Lys(3)-PAspDET](D₂O): δ 1.49 (m, 2H, CH₂), 2.28 (brs, 2H, CH₂), 3.13-3.76 (m, 2H, CH₂), 4.38 (brs, 2H, CH₂), 8.32 (s, 3H, aryl CH).

Table 17 Shows the characterization obtained for Star-6 arm initiator-PAspDET of formula (22).

TABLE 17

| Compound | R1 | DP_{PAspDET}^{a} | Mw^{a} (kDa) | [symbol] |
|---|---|---|---|---|
| CP55 | 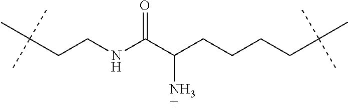 | 127 | 26.9 | 1.08 |

^{a}Determined by SEC.
Mw and DP refer to weight average molar mass and degree of polymerization respectively. [symbol]
represent polydispersity as determined by SEC-MALS software analysis.

### Example 11. Potentiometric Titration

The pKa of a cationic polymer is determined by acid-base titration, measuring the pH of the solution throughout the process. The pKa is then obtained from the titration graph.

To carry out the measurement, a 1 mg/mL solution of the cationic polymer is prepared in Milli-Q water and a known quantity of HCl 0.1M is added until the pH of the solution is around 2. At this point, the titration is performed with NaOH 0.2 M using an automatic Methrom 916 titouch potentiometer with a Dosin 800 dispenser. The titration speed is set to 0.1 mL/min with a signal drift of 50 mV/min. The titration is completed when the pH reaches 12.

The relationship between pH and the protonation degree of polycations was calculated from the obtained titration curves. Furthermore, pK=pH+log [α/(1−α)] values were plotted against 1−α, where K is the effective dissociation constant.

### Example 12. Polyplex Formation and Analysis

### Example 12A. Polyplex Formulation Procedure 1

Polyplex formation is prepared in-situ (mixing in a pipette) and added to the assay plate.

Polyplex formulations are named as “PX_{CPx_ratio}^{nuc}”, wherein “CPx” corresponds to the compound nomenclature as given above, which is used to form the polyplex; wherein “ratio” refers to the N/P ratio; and wherein “nuc” refers to the type of nucleic acid: pDNA or clDNA.

The sequence of the clDNA according to SEQ ID NO. 1, in the examples, is that of Table 18.

TABLE 18
Sequence of the clDNA (SEQ ID NO. 1)
SEQ ID NO: 1; linear closed DNA (clDNA) synthetic construct (artificial)
with 5580 nucleotides.

| | |
|---|---|
| gcgtataatg gactattgtg tgctgatagg tctcgccctg atttaaatcg tcgaccgtta | 60 |
| cataacttac ggtaaatggc ccgcctggct gaccgcccaa cgacccccgc ccattgacgt | 120 |
| caataatgac gtatgttccc atagtaacgc caatagggac tttccattga cgtcaatggg | 180 |
| tggagtattt acggtaaact gcccacttgg cagtacatca agtgtatcat atgccaagta | 240 |
| cgccccctat tgacgtcaat gacggtaaat ggcccgcctg gcattatgcc cagtacatga | 300 |
| ccttatggga ctttcctact tggcagtaca tctacgtatt agtcatcgct attaccatgg | 360 |
| tgatgcggtt ttggcagtac atcaatgggc gtggatagcg gtttgactca cggggatttc | 420 |
| caagtctcca ccccattgac gtcaatggga gtttgttttg gcaccaaaat caacgggact | 480 |
| ttccaaaatg tcgtaacaac tccgccccat tgacgcaaat gggcggtagg cgtgtacggt | 540 |
| gggaggtcta tataagcaga gctcgtttag tgaaccgtca gatcgcctgg agacgccatc | 600 |
| cacgctgttt tgacctccat agaagacacc gggaccgatc cagcctccgg actctagagg | 660 |
| atccggtact cgaggaactg aaaaaccaga aagttaactg gtaagtttag tctttttgtc | 720 |
| ttttatttca ggtcccggat ccggtggtgg tgcaaatcaa agaactgctc ctcagtggat | 780 |
| gttgccttta cttctaggcc tgtacggaag tgttacttct gctctaaaag ctgcggaatt | 840 |
| gtacccgcgg cccgggatcc accggtcgcc accatggaag acgccaaaaa cataaagaaa | 900 |
| ggcccggcgc cattctatcc tctagaggat ggaaccgctg gagagcaact gcataaggct | 960 |
| atgaagagat acgccctggt tcctggaaca attgctttta cagatgcaca tatcgaggtg | 1020 |
| aacatcacgt acgcggaata cttcgaaatg tccgttcggt tggcagaagc tatgaaacga | 1080 |
| tatgggctga atacaaatca cagaatcgtc gtatgcagtg aaaactctct tcaattcttt | 1140 |
| atgccggtgt tgggcgcgtt atttatcgga gttgcagttg cgcccgcgaa cgacatttat | 1200 |
| aatgaacgtg aattgctcaa cagtatgaac atttcgcagc ctaccgtagt gtttgtttcc | 1260 |
| aaaaaggggt tgcaaaaaat tttgaacgtg caaaaaaaat taccaataat ccagaaaatt | 1320 |
| attatcatgg attctaaaac ggattaccag ggatttcagt cgatgtacac gttcgtcaca | 1380 |
| tctcatctac ctcccggttt taatgaatac gattttgtac cagagtcctt tgatcgtgac | 1440 |
| aaaacaattg cactgataat gaattcctct ggatctactg ggttacctaa gggtgtggcc | 1500 |
| cttccgcata gaactgcctg cgtcagattc tcgcatgcca gagatcctat ttttggcaat | 1560 |
| caaatcattc cggatactgc gattttaagt gttgttccat tccatcacgg ttttggaatg | 1620 |
| tttactacac tcggatattt gatatgtgga tttcgagtcg tcttaatgta tagatttgaa | 1680 |
| gaagagctgt ttttacgatc ccttcaggat tacaaaattc aaagtgcgtt gctagtacca | 1740 |
| accctatttt cattcttcgc caaaagcact ctgattgaca aatacgattt atctaattta | 1800 |
| cacgaaattg cttctggggg cgcacctctt tcgaaagaag tcggggaagc ggttgcaaaa | 1860 |
| cgcttccatc ttccagggat acgacaagga tatgggctca ctgagactac atcagctatt | 1920 |
| ctgattacac ccgaggggga tgataaaccg ggcgcggtcg gtaaagttgt tccatttttt | 1980 |
| gaagcgaagg ttgtggatct ggataccggg aaaacgctgg gcgttaatca gagaggcgaa | 2040 |
| ttatgtgtca gaggacctat gattatgtcc ggttatgtaa acaatccgga agcgaccaac | 2100 |
| gccttgattg acaaggatgg atggctacat tctggagaca tagcttactg ggacgaagac | 2160 |
| gaacacttct tcatagttga ccgcttgaag tctttaatta aatacaaagg atatcaggtg | 2220 |
| gcccccgctg aattggaatc gatattgtta caacacccca acatcttcga cgcgggcgtg | 2280 |
| gcaggtcttc ccgacgatga cgccggtgaa cttcccgccg ccgttgttgt tttggagcac | 2340 |
| ggaaagacga tgacggaaaa agagatcgtg gattacgtcg ccagtcaagt aacaaccgcg | 2400 |
| aaaaagttgc gcggaggagt tgtgtttgtg gacgaagtac cgaaaggtct taccggaaaa | 2460 |
| ctcgacgcaa gaaaaatcag agagatcctc ataaaggcca agaagggcgg aaagtccaaa | 2520 |
| ttgtaaagcg gccgcgggga tccagacatg ataagataca ttgatgagtt tggacaaacc | 2580 |
| acaactagaa tgcagtgaaa aaaatgcttt atttgtgaaa tttgtgatgc tattgcttta | 2640 |
| tttgtaacca ttataagctg caataaacaa gttaacaaca acaattgcat tcattttatg | 2700 |
| tttcaggttc agggggaggt gtgggaggtt ttttagtcga ccatttaaat cggtctcgag | 2760 |
| ggtatcagca cacaattgcc cattatacgc gcgtataatg ggcaattgtg tgctgatacc | 2820 |
| ctcgagaccg atttaaatgg tcgactaaaa aacctcccac acctccccct gaacctgaaa | 2880 |
| cataaaatga atgcaattgt tgttgttaac ttgtttattg cagcttataa tggttacaaa | 2940 |
| taaagcaata gcatcacaaa tttcacaaat aaagcatttt tttcactgca ttctagttgt | 3000 |
| ggtttgtcca aactcatcaa tgtatcttat catgtctgga tccccgcggc cgctttacaa | 3060 |
| tttggacttt ccgcccttct tggcctttat gaggatctct ctgatttttc ttgcgtcgag | 3120 |
| ttttccggta agacctttcg gtacttcgtc cacaaacaca actcctccgc gcaacttttt | 3180 |
| cgcggttgtt acttgactgg cgacgtaatc cacgatctct ttttccgtca tcgtctttcc | 3240 |
| gtgctccaaa acaacaacgg cggcgggaag ttcaccggcg tcatcgtcgg gaagacctgc | 3300 |
| cacgcccgcg tcgaagatgt tggggtgttg taacaatatc gattccaatt cagcgggggc | 3360 |
| cacctgatat cctttgtatt taattaaaga cttcaagcgg tcaactatga agaagtgttc | 3420 |
| gtcttcgtcc cagtaagcta tgtctccaga atgtagccat ccatccttgt caatcaaggc | 3480 |
| gttggtcgct tccggattgt ttacataacc ggacataatc ataggtcctc tgacacataa | 3540 |
| ttcgcctctc tgattaacgc ccagcgtttt cccggtatcc agatccacaa ccttcgcttc | 3600 |
| aaaaaatgga acaactttac cgaccgcgcc cggtttatca tccccctcgg gtgtaatcag | 3660 |
| aatagctgat gtagtctcag tgagcccata tccttgtcgt atccctggaa gatggaagcg | 3720 |
| ttttgcaacc gcttccccga cttctttcga aagaggtgcg cccccagaag caatttcgtg | 3780 |
| taaattagat aaatcgtatt tgtcaatcag agtgcttttg gcgaagaatg aaaatagggt | 3840 |
| tggtactagc aacgcacttt gaattttgta atcctgaagg gatcgtaaaa acagctcttc | 3900 |
| ttcaaatcta tacattaaga cgactcgaaa tccacatatc aaatatccga gtgtagtaaa | 3960 |
| cattccaaaa ccgtgatgga atggaacaac acttaaaatc gcagtatccg gaatgatttg | 4020 |
| attgccaaaa ataggatctc tggcatgcga gaatctgacg caggcagttc tatgcggaag | 4080 |
| ggccacaccc ttaggtaacc cagtagatcc agaggaattc attatcagtg caattgtttt | 4140 |
| gtcacgatca aaggactctg gtacaaaatc gtattcatta aaaccgggag gtagatgaga | 4200 |
| tgtgacgaac gtgtacatcg actgaaatcc ctggtaatcc gttttagaat ccatgataat | 4260 |
| aattttctgg attattggta attttttttg cacgttcaaa attttttgca accccttttt | 4320 |
| ggaaacaaac actacggtag gctgcgaaat gttcatactg ttgagcaatt cacgttcatt | 4380 |
| ataaatgtcg ttcgcgggcg caactgcaac tccgataaat aacgcgccca acaccggcat | 4440 |
| aaagaattga agagagtttt cactgcatac gacgattctg tgatttgtat tcagcccata | 4500 |
| tcgtttcata gcttctgcca accgaacgga catttcgaag tattccgcgt acgtgatgtt | 4560 |
| cacctcgata tgtgcatctg taaaagcaat tgttccagga accagggcgt atctcttcat | 4620 |
| agccttatgc agttgctctc cagcggttcc atcctctaga ggatagaatg gcgccgggcc | 4680 |
| tttctttatg tttttggcgt cttccatggt ggcgaccggt ggatcccggg ccgcgggtac | 4740 |
| aattccgcag cttttagagc agaagtaaca cttccgtaca ggcctagaag taaaggcaac | 4800 |
| atccactgag gagcagttct ttgatttgca ccaccaccgg atccgggacc tgaaataaaa | 4860 |
| gacaaaaaga ctaaacttac cagttaactt tctggttttt cagttcctcg agtaccggat | 4920 |
| cctctagagt ccggaggctg gatcggtccc ggtgtcttct atggaggtca aaacagcgtg | 4980 |
| gatggcgtct ccaggcgatc tgacggttca ctaaacgagc tctgcttata tagacctccc | 5040 |
| accgtacacg cctaccgccc atttgcgtca atggggcgga gttgttacga cattttggaa | 5100 |
| agtcccgttg attttggtgc caaaacaaac tcccattgac gtcaatgggg tggagacttg | 5160 |
| gaaatccccg tgagtcaaac cgctatccac gcccattgat gtactgccaa aaccgcatca | 5220 |
| ccatggtaat agcgatgact aatacgtaga tgtactgcca agtaggaaag tcccataagg | 5280 |
| tcatgtactg ggcataatgc caggcgggcc atttaccgtc attgacgtca atagggggcg | 5340 |
| tacttggcat atgatacact tgatgtactg ccaagtgggc agtttaccgt aaatactcca | 5400 |
| cccattgacg tcaatggaaa gtccctattg gcgttactat gggaacatac gtcattattg | 5460 |
| acgtcaatgg gcgggggtcg ttgggcggtc agccaggcgg gccatttacc gtaagttatg | 5520 |
| taacggtcga cgatttaaat cagggcgaga cctatcagca cacaatagtc cattatacgc | 5580 |

In the following examples, a pDNA (purchased from PlasmidFactory, with reference PF461 (pCMV-luc)), containing 6233 bp expressing luciferase), commercial mRNA(luc) (purchased form Trilink Biotechnologies), commercial pDNA(GFP) (purchased from Akron Biotech) and a clDNA according to SEQ ID NO. 1 which was obtained according to standard molecular biology methods, such as the one disclosed in Heinrich, M. et al. “Linear closed mini DNA generated by the prokaryotic cleaving-joining enzyme TeIN is functional in mammalian cells”, J Mol Med, 2002, vol. 80, pp. 648-654. Are used as nucleic acid of interest to cover different sizes and protein expression.

Polyplex formulations to study toxicity and transfection capacity were prepared in-situ (mixing in a pipette) as follows: 100, 200 or 1000 ng of pDNA (pDNA was purchased from PlasmidFactory, with reference PF461 (pCMV-luc)), containing 6233 bp expressing luciferase or clDNA according to SEQ ID NO. 1 expressing luciferase and the calculated amount of polymer at indicated charge-ratio (+/−) or amine to phosphate ratio (N/P) were diluted in separate tubes in 10 μL of Hepes 20 mM buffer. Only protonatable nitrogens, not amide nitrogens, were considered in the +/−ratio and N/P ratio calculations. For polyplex formation, the nucleic acid and the polymer solution were mixed by rapidly pipetting up and down (ten times) and incubated for 20 min at RT. Then the polyplexes formed were characterized by DLS to determine the size and the Z-potential (Table 18).

Those samples formulated by this first method were prepared in a similar manner for its in-vitro testing but exchanging the Hepes buffer by NaCl 150 mM. After 48 hours of incubation the toxicity and transfection ability will be evaluated. The ratios studied for each polymer were N/P 5 with 100 ng of clDNA and NP 10, 30 and 100 with 200 ng of clDNA. As a positive control for the transfection jet PEI (Polyplus-transfection S.A, Illkirch, France) (Ref Polyplus: 101-10N) was used at nitrogen to phosphorus ratio (NP5) 100 ng of pDNA/clDNA for HaCaT and at NP8 100 ng of pDNA/clDNA for BJ cells. Cell transfection was performed using jetPEI® according to the manufacturer's instructions. jetPEI® is a powerful reagent that ensures robust, effective and reproducible DNA transfection into mammalian cells with low toxicity. jetPEI® is mainly composed of a linear polyethylenimine manufactured at Polyplus-transfection. jetPEI® is provided as a 7.5 mM solution in sterile and apyrogenic water (expressed as concentration of nitrogen residues)

### Example 12A.1. Size and Z-Pot of the Polyplexes

After 20 min of stabilization, the size and Z potential of the polyplexes formed with clDNAat N/P ratio=30 were performed using a Malvern ZetasizerNanoZS instrument, equipped with a 532 nm laser at a fixed scattering angle of 173°. After polyplex formation, the solutions were allowed to stabilize for 20 min, and 20 μl were measured using a quartz glass high performance cuvette (Hellma Analytics). Size distribution was measured (diameter, nm) with n>3 measurements, results are shown in table 19.

TABLE 19

| | Hydrodynamic | |
|---|---|---|
| Polyplex | diameter/nm | Z-potential |
| PX5_{cp5 30}^{cIDNA} | 22 ± 2.53 | 22 ± 4.35 |
| PX10_{cp10 30}^{cIDNA} | 26.4 ± 2.19 | 30.4 ± 8.54 |
| PX7_{cp7 30}^{cIDNA} | 23.5 ± 3.06 | 22.8 ± 3.99 |
| PX9_{cp9 30}^{cIDNA} | 23 ± 7.07 | 36.4 ± 3.15 |
| PX11_{cp11 30}^{cIDNA} | 19.4 ± 8.66 | 32.1 ± 6.30 |
| PX12_{cp12 30}^{cIDNA} | 23.5 ± 2.40 | 20.6 ± 2.81 |
| PX13_{cp13 30}^{cIDNA} | 16.2 ± 6.01 | 31.6 ± 11.4 |
| PX14_{cp14 30}^{cIDNA} | 23.8 ± 2.53 | 29.7 ± 6.75 |
| PX17_{cp17 30}^{cIDNA} | 21.9 ± 3.36 | 34.7 ± 8.54 |
| PX19_{cp19 30}^{cIDNA} | 25.5 ± 2.97 | 20.8 ± 6.76 |
| PX20_{cp20 30}^{cIDNA} | 27.2 ± 2.85 | 19.3 ± 3.91 |
| PX21_{cp21 30}^{cIDNA} | 25.8 ± 5.59 | 28.3 ± 1.24 |
| PX22_{cp22 30}^{cIDNA} | 28.2 ± 9.18 | 32.9 ± 3.21 |
| PX23_{cp23 30}^{cIDNA} | 25.3 ± 7.92 | 24.8 ± 10.6 |
| PX30_{cp30 30}^{cIDNA} | 38.2 ± 3.4 | 20.8 ± 2.7 |
| PX31_{cp31 30}^{cIDNA} | 29.4 ± 7 | 22.9 ± 3.8 |

All polyplexes formed at this N/P ratio showed sizes around 20-30 nm of hydrodynamic diameter and a Z-Potential between 20 and 35 mV.

### Example 12A.2. Complexation/Disassembly Experiments

In addition, the complexation efficacy and possible presence of free pDNA in the polyplexes were assessed using an electrophoresis gel as first screening method. To perform the electrophoresis, E-gel Power Snap Electrophoresis Device and E-Gel Power Snap Camera (Invitrogen) was used. 1.2% agarose gels prepared that include the SYBR safe DNA marker (E-Gel® 1.2% with SYBR safe, Invitrogen) were used.

The complexation efficiency of the polyplexes (20 μl) at different NPs (5, 10, 30) were evaluated, and also the disassembly of the polyplexes at NP30 in the presence of low heparin (0.075 IU/ml) and high heparin (200 IU/ml) concentration (PanReacAppliChem, Spain). For the low concentration, 0.1 μl of a 15 IU/ml heparin solution was added to the 20 μl of already formed polyplex, and for the high concentration, 0.8 μl of a 5000 IU/ml heparin solution to the 20 μl of polyplex was added. Once the gel is loaded (20 μl/well), select the equipment protocol according to the type of gel used (in our case, E-Gel 0.8-2% protocol approximately 40 min, although the time can be modified according to the samples).

Table 20. Complexation and disassembly experiments for representative of polyplexes synthesised.

TABLE 20

| Compound | Complexation | Dissassembly test |
|---|---|---|
| PX32_{cp5 30}^{pDNA} | YES | YES (high heparin) |
| PX33_{cp10 30}^{pDNA} | YES | YES (high heparin) |
| PX34_{cp7 30}^{pDNA} | YES | YES (high heparin) |
| PX35_{cp9 30}^{pDNA} | YES | YES (high heparin) |
| PX36_{cp11 30}^{pDNA} | YES | YES (high heparin) |
| PX37_{cp12 30}^{pDNA} | YES | YES (high heparin) |
| PX38_{cp13 30}^{pDNA} | YES | YES (high heparin) |
| PX39_{cp14 30}^{pDNA} | YES | YES (high heparin) |
| PX40_{cp17 30}^{pDNA} | YES | YES (high heparin) |
| PX41_{cp19 30}^{pDNA} | YES | YES (high heparin) |
| PX42_{cp20 30}^{pDNA} | YES | YES (high heparin) |
| PX43_{cp21 30}^{pDNA} | YES | YES (high heparin) |
| PX44_{cp22 30}^{pDNA} | YES | YES (high heparin) |
| PX45_{cp23 30}^{pDNA} | YES | YES (high heparin) |
| PX46_{cp30 30}^{pDNA} | YES | YES (high heparin) |
| PX47_{cp31 30}^{pDNA} | YES | YES (high heparin) |

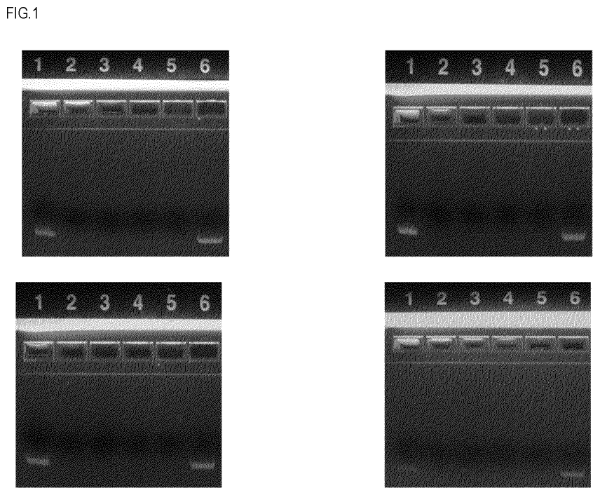

In all cases, no free pDNA is observed at the different NPs or at low concentrations of heparin. However, at high concentration of heparin, free pDNA signal was observed due to the competition between heparin and pDNA to bind to the polymer, showing the ability of the polymer to release their cargo (Representative image of the gels can be observed in FIG. 1).

### Example 12B. Polyplex Formulation Procedure 2

The microfluidic device is placed in a laminar flow hood to avoid possible contamination of the samples and all the polymers used in this formulation step were previously sterilized by passing them through 0.22 μm PES filter. In this first screening, the stability and formation of the polyplexes using different NP ratios (10 and 30) in PBS pH7.4 were studied. For this study, 0.2 μg of pDNA and the polymer compound (NVV) were used. For the formation of the polyplexes, pDNA solution (100 μl, 0.2 μg) and polymer solution (100 μl, mass calculated as shown in polyplex formation section) were loaded into 1 ml plastic syringes (BD Plastipak™, Spain). The two programmable infusion syringe pumps (NE-4000, Syringe Pump, USA) were then used for fluidic injection and control according to the desired flow-rate ratio (300 μl/min) of the side streams to the central stream. Then, the final polyplex solution was collected and allowed to stabilize for 20 min. Based on these stability studies, optimal NP ratio was identified (30), and corresponding polyplexes formed by NVVs were formed.

For all microfluidics experiments, the microfluidic device was at room temperature. Any additional detail about size and stability of polyplexes as well as about complexation/disassembly experiments are described below.

### Example 12B.1. Microfluidic Device and Setup

A microfluidic device was purchased in Little Things Factory GmbH (Germany). The system consists in two connected reactors made of borosilicate glass: first reactor (LTF-MS) presents 2 inlet-channels (one for the DNA and the other for the polymer) and 1 outlet-channel, Volume 0.2 ml, Channel size: 1 mm, 0.5-20 ml/min/channel, Not sensitive to blockage. Size: 115×60×6 mm (l, w, h). The second reactor (LTF-VS) has 1 inlet-channel (connected to the outlet-channel of the first reactor) and 1 outlet-channel, Volume 1.1 ml, Channel size: 1 mm. Size: 115×60×6 mm (l, w, h). First reactor is employed for the mix and formation of the polyplexes, and the second reactor is used to increase the residence time.

In addition, two programmable pumps control the fluid flow rates of the syringes (NE-4000 Programmable 2 Channel Syringe Pump, Syringe Pump, USA). The system accepts infusion rates from 1.436 μL/hr (1 mL syringe) to 7515 mL/hr (60 mL syringe).

This methodology provides reproducibility to the formation of polyplexes as well as the possibility of scaling up the process.

### Example 12B.2. Optimization of Different Parameters for Microfluidics Formulation

Different rates and reaction times (150, 300, and 600 μL/min) were evaluated, as well as various buffers with different ionic strength (Hepes 20 mM, NaCl 150 mM, and a mixture of Hepes 20 mM and NaCl 130 mM). For this study we used 1 ml syringe (BD Plastipak™, Spain). As an example, we used a commercial reference polymer which is an end capped acetyl n-butyl-Poly-L-Aspartic-Diethylenetriamine (Degree of polymerization of PAsp=50) (Sunbright AS50-DT-A (Internal code: CXP15D_4) (acquired from NOF therapeutics) and pDNA (pCMV-luc, PlasmidFactory, Germany), using nitrogen to phosphorus ratio (NP30). The polymer solution (100 μl) was introduced via one of the inlet-channel, and the DNA solution (100 μl) was introduced via the other inlet-channel of the first reactor. Then, the final polyplex solution was collected and allowed to stabilize for 20 min before measuring the size by DLS (Malvern Panalytical, Spain).

We observe that using the 3 flow rates, stable polyplexes are formed, whose size is adequate and their correlation coefficient indicates a good signal. The optimization was carried out taking a close look to the quality indicators of the measurement and sample homogeneity. the intermediate flow rate was selected for further formulation of polyplexes(300 μl/min) in order to have an adequate control over the size and the formation of the polyplex at room temperature. The hydrodinamic diameter (measured by DLS) of the polyplexes formed using this procedure was around 30 nm.

### Example 12C. Stability of Polyplexes

Stability of polyplexes is a paramount aspect on developing efficient therapies. Ensuring mid-long term stability of the drug product formulation is followed by a panel assay to mimic the physiological conditions that the drug will meet following the administration route where they need be stable during circulation to the target site of action. It is well known that polyplexes displaying positive surface charges undergo salt-induced agglomeration which might cause inaccurate cell biology evaluation and severe toxicity issues when applied systemically. Initial stability studies are currently under development during the present project, they are aimed to monitor polyplex particle characteristics (size); in addition, more studies must be further developed and implemented to evaluate DNA-polymer concentrations (complexed vs. free)—critical action to be performed during hit to lead stage and become a routinary QC assay. For the stability measurements, the polyplexes were kept in the fridge during the experiment and the stability of the polyplexes was measured at different times.

### Example 12C.1 Stability in Biologically Relevant Medium

The stability at different times and the formation of the polyplexes by microfluidics (as reported above), using NP=30 ratio in PBS pH7.4 were studied. For this experiment, 3 μg of pDNAluc (pCMV-luc, PlasmidFactory, Germany) and the polymers CP36 and CP19 were used. The final concentrations of the polymers in the polyplexes were 0.335 mg/ml for CP36, and 0.202 mg/ml for CP19. The final polyplex solution (200 μl) was allowed to stabilize for 20 min before measuring the size by DLS (Malvern Panalytical, Spain). The polyplexes were kept in the fridge during the experiment, and the stability of the polyplexes was measured at different times.

As shown in table 21, the presence of the sarcosine in the structure of the polymer CP36 provided stability to the polyplex in solution up to 1 month, maintaining a constant size over time and avoiding aggregation.

TABLE 21

| | D(n)/nm | D(n)/nm | D(n)/nm | D(n)/nm |
|---|---|---|---|---|
| Polyplex | t = 0 h | t = 24 h | t = 6 days | t = 1 month |
| PX41_{cp19 30}^{pDNA} | 1280 ± 160 | N/A | N/A | N/A |
| PX48_{cp36 30}^{pDNA} | 69 ± 11 | 55 ± 19 | 38 ± 6 | 39 ± 20 |

D(n) stands for hydrodynamic diameter measured by DLS;
N/A stands for not able to be measured because aggregation was present

### Example 12C.2 Stability in Different Buffers: Impact of Salts on Polyplex Aggregation

Polyplex stability in different buffers (pure water, Hepes 20 mM, and PBS pH7.4) using NP=30 ratio was studied at different times. For this experiment, 3 μg of pDNAluc (pCMV-luc, PlasmidFactory, Germany) and the polymers CP38 and CP19 were used. The final concentrations of the polymers in the polyplexes were 0.335 mg/ml for CP38, 0.202 mg/ml for CP19. The final polyplex solution (200 μl) was allowed to stabilize for 20 min before measuring the size by DLS (Malvern Panalytical, Spain). The polyplexes were kept in the fridge during the experiment, and the stability of the polyplexes was measured at different times.

As shown in table 22, the presence of salts in the buffer (PBS pH7.4) promotes the destabilization of the polyplexes without sarcosine in their structure (CP19), while the polymer with sarcosine (CP38) remains stable. However, in the buffers with less or without salts (water and hepes 20 mM) all the polyplexes maintained a constant size over time, avoiding aggregation.

TABLE 22

| | | D(n)/nm | D(n)/nm | D(n)/nm | D(n)/nm |
|---|---|---|---|---|---|
| Solvent | Polyplex | t = 0 h | t = 24 h | t = 2 days | t = 5 days |
| Water | PX48_{cp36 30}^{pDNA} | 46 ± 6 | 46 ± 3 | 48 ± 2 | 49 ± 5 |
| Water | PX41_{cp19 30}^{pDNA} | 34 ± 3 | 76 ± 2 | 38 ± 4 | 37 ± 2 |
| Water | PX32_{cp5 30}^{pDNA} | 30 ± 5 | 36 ± 6 | Not measured | 31.6 ± 8.71 |
| Water | PX34_{cp7 30}^{pDNA} | 38 ± 3 | 39 ± 5 | Not measured | 35.6 ± 5.24 |
| Water | PX45_{cp23 30}^{pDNA} | 38 ± 5 | 41 ± 5 | Not measured | 42.1 ± 2.62 |
| HEPES | PX48_{cp36 30}^{pDNA} | 43 ± 9 | 47 ± 2 | Not measured | 48 ± 7 |
| 20 mM | | | | | |
| HEPES | PX41_{cp19 30}^{pDNA} | 38 ± 7 | 28 ± 7 | Not measured | 47 ± 5 |
| 20 mM | | | | | |
| PBS | PX48_{cp36 30}^{pDNA} | 69 ± 11 | 55 ± 20 | 39 ± 6 | 38 ± 18 |
| PBS | PX41_{cp19 30}^{pDNA} | 1280 ± 55 | N/A | N/A | N/A |

D(n) stands for hydrodynamic diameter measured by DLS; N/A stands for not able to be measured because aggregation was present.

At this point, is worth to mention that despite complexing polymers that has a molar ratio PAA1+PAA3/PAA2+PAA4 from 10/80 to 60/40 can stabilize the polyplexes, they show a very limited transfection efficiency, being an example of the necessity to adjust those ratios above 60/40. Examples of this complexing polymers are depicted above. i.e.: CP30, CP32, CP33, CP34, CP35 and CP36.

### Example 13. In-Vitro Biological Studies (I)

### Example 13A. Cell Culture

HaCaT cell line was obtained from CLS® (300493) and maintained in DMEM Glutamax (Gibco Ref:6195-026) supplemented with 10% FBS (HyClone Ref: SV30160.03) using standard tissue culture conditions. BJ cell line was obtained from ATCC® (CRL-2522) and maintained in EMEM (ATCC Ref:30-2003) supplemented with 10% FBS (HyClone Ref: SV30160.03) using standard tissue culture conditions. In both cases, cells were routinely maintained at 37° C. in a humidified atmosphere with 5% CO₂ and media was replaced every 2-3 days and underwent passaging once cells reached 80% cell confluence. Cell density (6000 cells/well for HaCat and 4500 cells/well for Fibroblasts BJ) was optimized to reach exponential growth and adequate readouts

To perform cytotoxicity and transfection efficiency experiments, cells were seeded in sterile 96-white well microtiter plates with transparent bottom (ViewPlate TC Ref:6005181 Perkin-Elmer) in 80 μl of complete medium at the density of 6000 cell/well and 4500 cell/well in HaCaT and BJ cells respectively. After 24 h of incubation 20 μl of polyplexes were added to study cytotoxicity and cellular transfection in independent plates.

### Example 13B. ATP Evaluation for Cell Toxicity Evaluation

After 48 h post-incubation, cellular viability was evaluated by determining the intracellular ATP content using the ATP1Step Kit (Perkin-Elmer #6016731) as described by the manufacturer. After incubation time, aliquots of 50 μl of cells were taken in duplicate in a black 96-well plate and mixed with 50 μl of ATP quantification reagent. ATP concentration was determined by reading chemical luminescence using the Victor Nivo plate reader. Viability was expressed as the percentage of the signal relative to non-treated cells.

### Example 13C. Luciferase Assay

After 48 h post-incubation, 20 μl of Bright-Glo Luciferase Assay System (Ref. E2610 Promega) was added to each well following manufacturer instructions. After 2 minutes of incubation at room temperature luciferase activity was measured using a Ensight Plate Reader (Perkin Elmer). Data was represented as luminescence relative to the cell viability and as the percentage of transfection relative to the positive control of transfection.

First of all, a first screening of polyplexes in HaCaT cells was performed. After that, we selected some polyplexes for further screening in other cell lines as fibro BJ.

The sequence of the clDNA indicated in this example, SEQ ID NO. 1, is that of Table 18 above.

Table 23 shows cell viability and transfection efficiency in HaCaT cells for representative synthesised polyplexes.

TABLE 23

| Polyplex | ng cIDNA | Cell viability | Transfection |
|---|---|---|---|
| PX5_{cp530}^{cIDNA} | 100 | 67.12 ± 4.50 | 100.03 ± 18.96 |
| PX10_{cp10 5}^{cIDNA} | 100 | 99.62 ± 15.60 | 15.69 ± 7.14 |
| PX10_{cp10 10}^{cIDNA} | 200 | 96.59 ± 13.02 | 20.90 ± 4.89 |
| PX10_{cp10 30}^{cIDNA} | 200 | 95.44 ± 8.46 | 12.13 ± 1.66 |
| PX10_{cp10 100}^{cIDNA} | 200 | 87.12 ± 13.47 | 35.72 ± 3.42 |
| PX7_{cp7 10}^{cIDNA} | 200 | 87.94 ± 6.29 | 57.13 ± 33.73 |
| PX7_{cp7 30}^{cIDNA} | 200 | 60.33 ± 1.62 | 380.13 ± 23.26 |
| PX9_{cp9 30}^{cIDNA} | 150 | 74.91 ± 13.74 | 15.88 ± 11.31 |
| PX11_{cp11 5}^{cIDNA} | 100 | 100.00 ± 2.66 | 2.25 ± 0.51 |
| PX11_{cp11 10}^{cIDNA} | 200 | 91.06 ± 0.66 | 12.64 ± 4.65 |
| PX11_{cp11 30}^{cIDNA} | 200 | 84.1 ± 2.88 | 27.80 ± 4.86 |
| PX12_{cp12 30}^{cIDNA} | 150 | 70.39 ± 19.08 | 15.56 ± 9.38 |
| PX13_{cp13 30}^{cIDNA} | 150 | 72.06 ± 14.78 | 20.76 ± 19.99 |
| PX14_{cp14 30}^{cIDNA} | 150 | 72.83 ± 13.58 | 12.16 ± 13.05 |
| PX17_{cp17 30}^{cIDNA} | 150 | 58.12 ± 33.24 | 132.4 ± 59.18 |
| PX18_{cp18 30}^{cIDNA} | 150 | 88.56 ± 5.6 | 459.2 ± 314.6 |
| PX20_{cp20 30}^{cIDNA} | 150 | 90 ± 64 | 44 ± 3 |
| PX21_{cp21 30}^{cIDNA} | 150 | 79.54 ± 12.08 | 56.3 ± 52.5 |
| PX22_{cp22 30}^{cIDNA} | 150 | 75 ± 19 | 40.4 ± 8 |
| PX23_{cp23 30}^{cIDNA} | 150 | 74 ± 14.77 | 149.6 ± 144.3 |

Table 24 shows cell viability and transfection efficiency in fibroblasts BJ cells for selected polyplexes.

TABLE 24

| Polyplex | Cell viability | Transfection efficiency |
|---|---|---|
| PX10_{cp10 5}^{cIDNA} | 76.73 ± 0.66 | 23.03 ± 0.86 |
| PX11_{cp11 10}^{cIDNA} | 78.63 ± 7.06 | 62.42 ± 17.27 |

### Example 14. Comparison of Linear and Star NVV in Terms of Cell Viability and Transfection Efficiency

A face to face comparison between star-based selected polyplexes (PX23_{CP23 30}^{clDNA}, PX18_{CP18 30}^{clDNA}, PX17_{CP17 30}^{clDNA}, PX7_{CP7 30}^{clDNA} and PX5_{CP5 30}^{clDNA}) and standard linear-based PAsp-DET polyplexes (PX49_{Nbu-PAsp-DET(46)}^{clDNA}, PX50_{Nbu-PAsp-DET(43) 30}^{clDNA} and PX51_{Nbu-PAsp-DET(38) 30}^{clDNA*4}) was carried out in order to exemplify the improvement in transfection efficiency driven by this architectural modification in HeLa cells when delivering clDNA(luc). Polyplexes were formulated in the same manner as described in previous examples and the biological activity response evaluated in the same way.

The sequence of the clDNA indicated in this example, SEQ ID NO. 1, is that of Table 18 above.

TABLE 25

| Polyplex | ng cIDNA | Cell viability | Transfection |
|---|---|---|---|
| PX5_{cp5 30}^{cIDNA} | 100 | 67.1 ± 4.5 | 100.0 ± 19.0 |
| PX7_{cp7 30}^{cIDNA} | 200 | 60.3 ± 1.6 | 380.1 ± 23.3 |
| PX17_{cp17 30}^{cIDNA} | 150 | 58.1 ± 33.2 | 132.4 ± 59.2 |
| PX18_{cp18 30}^{cIDNA} | 150 | 88.6 ± 5.6 | 459.2 ± 314.6 |
| PX23_{cp23 30}^{cIDNA} | 150 | 74.0 ± 14.8 | 149.6 ± 144.3 |
| PX49_{Nbu-PAsp-DET(46)}^{cIDNA*} | 150 | 74 ± 12.2 | 90 ± 20 |
| PX50_{Nbu-PAsp-DET(43) 30}^{cIDNA*} | 150 | 63.7 ± 10.6 | 70 ± 12 |
| PX51_{Nbu-PAsp-DET(38) 30}^{cIDNA*} | 150 | 95.8 ± 25.1 | 84 ± 15 |

*Linear PAsp-DET were sinthesized and characterized in the same procedure shown in example 1B and 1C

As can be seen in the table above, the star-shaped polyplexes designed in the present invention, show better results in terms of transfection efficiency and toxicity. As an example, PX18_{cp18 30}^{clDNA} show a 6.5 fold increase in transfection efficiency when compared with a polyplex formed with a linear counterpart PX50_{Nbu-PAsp-DET(43) 30}^{clDNA*}. Also the cell viability is improved when comparing star-shaped vs linear polymers. For PX18_{cp18 30}^{clDNA} a 88.6% of cell viability while for PX50_{Nbu-PAsp-DET(43) 30}^{clDNA} the cell viability is 63.7%.

### Example 15. In-Vitro Biological Studies (II)

### Example 15A. Cell Culture

HeLa cells were cultured in DMEM high glucose with Glutamax (Gibco-Thermo Fisher #61965-059) supplemented with 10% of Fetal Bovine Serum (Hyclone #SV30160.03H1, provided by GE Healthcare Europe GmbH). Transfections were carried out on 96-well plates containing 10000 cells/well in a final volume of 100 μl, and cells were incubated 24 hours at 37° C. and 5% CO2. After 24 h, the medium was removed and refreshed with 90 μl of complete medium. The transfection mixtures were prepared using PBS and in the case of the positive control (JetPEI) manufacturer guidelines were followed (#101-10N, Polyplus Transfection), after 20 min of stabilization 10 μl of each formulation were added to the cells. After 24 hours cells were recovered and processed.

HEK293 (Human embryonic kidney) cells were cultured in DMEM high glucose (Gibco ref 61965-059)+10% FBS ((Hyclone #SV30160.03H1, provided by GE Healthcare Europe GmbH). Transfections were carried out on 96-well plates containing 10000 cells/well in a final volume of 100 μl, and cells were incubated 24 hours at 37° C. and 5% CO2. After 24 h, the medium was removed and refreshed with 90 μl of complete medium. The transfection mixtures were prepared using PBS and in the case of the positive control (JetPEI) manufacturer guidelines were followed (#101-10N, Polyplus Transfection), after 20 min of stabilization 10 μl of each formulation were added to the cells. After 24 hours cells were recovered and processed

### Example 15B. ATP Evaluation for Cell Toxicity Evaluation

After 24 h post-incubation, the medium was aspirated and 50 μl/well of ATPLite reagent (ATPLite PerkinElmer #6016731) were added. The plate was incubated 10 minutes at room temperature in the dark. Luminiscence was read spectrophotometrically using VictorNivo (PerkinElmer) and data was represented as the percentage of cell viability, taken untreated control cells as 100%.

### Example 15C. Luciferase Assay

After 24 h post-incubation, 100 μl of BrightGlo reagent (Promega #E2620) was added in each well following manufacturer instructions. After 5 minutes of incubation at room temperature luciferase activity was measured using VictorNivo (PerkinElmer). Data was represented as luminescence relative to the percentage of transfection relative to the positive control of transfection.

### Example 15D. Biological Activity of Polyplexes in HeLa Cells

The transfection efficiency and the cell viability of the polyplexes formed by CP46, CP53, CP50, and CP45 in HeLa cells were reported in the following table. The transfection data is represented as % of the positive control jetPEI® being the positive control 100% after 24 h of treatment, and cell viability is compared to non-treated (NT) cells, being the ATP content readout of NT cells equal to 100%.

TABLE 26
Cell viability and transfection in HeLa cells
with mRNA(luc) and pDNA(luc) (n = 2)

| | Genetic | | |
|---|---|---|---|
| Polyplex | material mass | Cell viability | Transfection |
| PX52_{cp46}_{_{—}}₈_{_{—}}^{mRNA} | 1 μg | 108 ± 37 | 8 ± 4 |
| PX53_{cp53}_{_{—}}₈_{_{—}}^{mRNA} | 1 μg | 102 ± 32 | 13 ± 3 |
| PX54_{cp50}_{_{—}}₈_{_{—}}^{mRNA} | 1 μg | 78 ± 5 | 38.1 ± 1.2 |
| PX55_{cp45}_{_{—}}₈_{_{—}}^{mRNA} | 1 μg | 77.3 ± 0.8 | 232 ± 130 |
| PX56_{cp46}_{_{—}}₁₅_{_{—}}^{mRNA} | 1 μg | 86 ± 2 | 35 ± 46 |
| PX57_{cp53}_{_{—}}₁₅_{_{—}}^{mRNA} | 1 μg | 94 ± 46 | 3.7 ± 0.6 |
| PX58_{cp50}_{_{—}}₁₅_{_{—}}^{mRNA} | 1 μg | 74 ± 26 | 6 ± 5 |
| PX59_{cp45}_{_{—}}₁₅_{_{—}}^{mRNA} | 1 μg | 96 ± 17 | 693 ± 818 |
| PX60_{cp46}_{_{—}}₈_{_{—}}^{pDNA} | 1 μg | 93.1 ± 1.0 | 8 ± 6 |
| PX61_{cp53}_{_{—}}₈_{_{—}}^{pDNA} | 1 μg | 104.7 ± 0.96 | 12 ± 13 |
| PX62_{cp50}_{_{—}}₈_{_{—}}^{pDNA} | 1 μg | 81. ± 7 | 67 ± 56 |
| PX63_{cp45}_{_{—}}₈_{_{—}}^{pDNA} | 1 μg | 77 ± 4 | 127 ± 109 |
| PX64_{cp46}_{_{—}}₁₅_{_{—}}^{pDNA} | 1 μg | 95 ± 12 | 5.5 ± 1.2 |
| PX65_{cp53}_{_{—}}₁₅_{_{—}}^{pDNA} | 1 μg | 66 ± 58 | 10 ± 3 |
| PX66_{cp50}_{_{—}}₁₅_{_{—}}^{pDNA} | 1 μg | 69 ± 32 | 89 ± 115 |
| PX67_{cp45}_{_{—}}₁₅_{_{—}}^{pDNA} | 1 μg | 67 ± 19 | 285 ± 393 |

### Example 15E. Biological Activity of Polyplexes in HEK293 Cells

The transfection efficiency and the cell viability of the polyplexes formed by CP46, CP53, CP50, and CP45 in HEK293 cells were reported in the following table. The transfection data is represented as % of the positive control jetPEI® being the positive control 100% after 24 h of treatment, and cell viability is compared to non-treated (NT) cells, being the ATP content readout of NT cells equal to 100%.

TABLE 27
Cell viability and transfection in HEK293
cells with mRNA and pDNA (n = 2)

| | Genetic | | |
|---|---|---|---|
| Polyplex | material mass | Cell viability | Transfection |
| PX52_{cp46}_{_{—}}₈_{_{—}}^{mRNA} | 1 μg | 82 ± 42 | 27 ± 31 |
| PX53_{cp53}_{_{—}}₈_{_{—}}^{mRNA} | 1 μg | 103 ± 32 | 48 ± 57 |
| PX54_{cp50}_{_{—}}₈_{_{—}}^{mRNA} | 1 μg | 84 ± 24 | 147 ± 184 |
| PX55_{cp45}_{_{—}}₈_{_{—}}^{mRNA} | 1 μg | 90.8 ± 0.4 | 1002 ± 491 |
| PX56_{cp46}_{_{—}}₁₅_{_{—}}^{mRNA} | 1 μg | 108 ± 43 | 19 ± 23 |
| PX57_{cp53}_{_{—}}₁₅_{_{—}}^{mRNA} | 1 μg | 120 ± 39 | 33 ± 40 |
| PX58_{cp50}_{_{—}}₁₅_{_{—}}^{mRNA} | 1 μg | 42 ± 42 | 97 ± 1178 |
| PX59_{cp45}_{_{—}}₁₅_{_{—}}^{mRNA} | 1 μg | 40.5 ± 0.5 | 2063 ± 1361 |
| PX60_{cp46}_{_{—}}₈_{_{—}}^{pDNA} | 1 μg | 98 ± 19 | 34 ± 43 |
| PX61_{cp53}_{_{—}}₈_{_{—}}^{pDNA} | 1 μg | 1073 ± 22 | 53. ± 67 |
| PX62_{cp50}_{_{—}}₈_{_{—}}^{pDNA} | 1 μg | 82 ± 28 | 215 ± 212 |
| PX63_{cp45}_{_{—}}₈_{_{—}}^{pDNA} | 1 μg | 83 ± 18 | 617 ± 598 |
| PX64_{cp46}_{_{—}}₁₅_{_{—}}^{pDNA} | 1 μg | 116 ± 36 | 26 ± 33 |
| PX65_{cp53}_{_{—}}₁₅_{_{—}}^{pDNA} | 1 μg | 103 ± 14 | 44 ± 58 |
| PX66_{cp50}_{_{—}}₁₅_{_{—}}^{pDNA} | 1 μg | 47.8 ± 45 | 241.3 ± 235.8 |
| PX67_{cp45}_{_{—}}₁₅_{_{—}}^{pDNA} | 1 μg | 33.5 ± 4.6 | 1052.8 ± 1175.7 |

### Example 15F. Biological Activity of Polyplexes in HEK293 Cells (100 ng and Compared with Lipofectamin2000®)

Cell transfection was performed using lipofectamine 2000® (ThermoFisher scientific, Spain. Ref No: 11668019) as positive control according to the manufacturer's instructions. Lipofectamine 2000® reagent delivers DNA or siRNA with excellent transfection performance for protein expression, gene silencing, and functional assays. The transfection efficiency and the cell viability of the polyplexes formed by CP58 (NP15 and NP30) and pDNA-GFP in HEK293 cells were studied at 24 h, 48 h, and 72 h. The results are reported in the following table. The transfection data is represented as % of the positive control Lipofectamine 2000® being the positive control 100%. Cell viability is compared to non-treated (NT) cells, being the ATP content readout of NT cells equal to 100%.

TABLE 28
Cell viability and transfection efficiency in HEK293
cells with pDNA-GFP (n = 2) vs lipofectamine2000

| | Time | Genetic | | Transfection |
|---|---|---|---|---|
| Polyplex | (h) | material mass | Cell viability | efficiency |
| PX68_{CP59}_{_{—}}₃₀_{_{—}}^{pDNA} | 24 | 100 ng | 80 ± 42 | 123 ± 28 |
| PX68_{CP59}_{_{—}}₃₀_{_{—}}^{pDNA} | 48 | 100 ng | 81 ± 9 | 356 ± 105 |
| PX68_{CP59}_{_{—}}₃₀_{_{—}}^{pDNA} | 72 | 100 ng | 87 ± 14 | 416 ± 245 |
| PX69_{CP59}_{_{—}}₁₅_{_{—}}^{pDNA} | 24 | 100 ng | 73 ± 19 | 121 ± 5 |
| PX69_{CP59}_{_{—}}₁₅_{_{—}}^{pDNA} | 48 | 100 ng | 88 ± 12 | 259 ± 121 |
| PX69_{CP59}_{_{—}}₁₅_{_{—}}^{pDNA} | 72 | 100 ng | 87.6 ± 0.7 | 254 ± 124 |

In all cases studied in the present example, the polyplexes were outperforming the lipofectamine 2000 transfection efficiency with good cell toxicity results.

### REFERENCES CITED IN THE APPLICATION

EP3331937
J Mol Med, 2002, vol. 80, pp. 648-654
T. W. Green and P. G. M. Wuts, Protective Groups in Organic Chemistry, Wiley, 3rd ed. 1999, Chapter 5 (pp. 369-451)
T. W. Green and P. G. M. Wuts, Protective Groups in Organic Chemistry, Wiley, 3rd ed. 1999, Chapter 7 (pp. 495-653)

### CLAUSES

For reasons of completeness, various aspects of the invention are set out in the following numbered clauses:

Clause 1. A compound of formula I, a pharmaceutically acceptable salt thereof, or any stereoisomer or mixtures of stereoisomers, either of the compound of formula (I) or of any of its pharmaceutically acceptable salts, comprising homo-polypeptides or random or block or graft co-polypeptides:

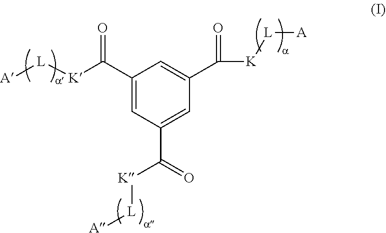

wherein A, A′ and A″ are each independently selected from a radical of formula II; and each of A, A′ and A″ sub-units may be same or different;

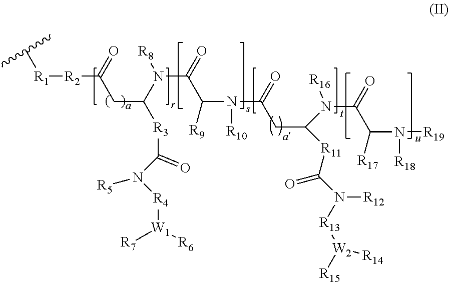

wherein the wavy line denotes the attaching point;
and though the repeating units defined by square brackets with their numerical value, r, s, t and u respectively in the formula II are shown in a particular order for convenience of description, the repeating units may be present in any order and the repeating units may be block or randomly present; and wherein each of the repeating units, may comprise blocks of monomer units which may be the same or different from each other;
wherein K, K′ and K″ are each independently selected from —O— and —NH—; L is selected from

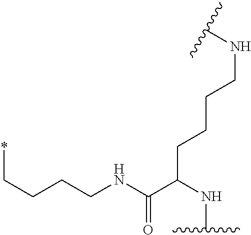 [SMILES: *CCCCNC(=O)C(CCCCN*)N*]

wherein α, α′ and α″ are an integer from 0 to 1; each wavy lines denote the attaching points to A, A′ or A″; and “*” denotes the attaching point to K, K′ or K″;
wherein R2 is selected from —O— and —NH—
wherein R1 is a biradical selected from the group consisting of (III) and (IV)

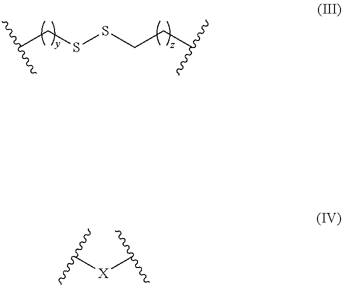

wherein the wavy lines denote the attaching points;
wherein y and z are integers independently ranging from 1 to 20;
X is a biradical selected from the group consisting of —NH—, —NH(C₁-C₆)alkyl-, —O—, —(C₁-C₆)alkyl-COO—, a straight or branched —(C₁-C₃₀)alkylene-, and a biradical selected from the group consisting of (V), (VI), (VII), (VIII), (IX), (X), (XI) and (XII)

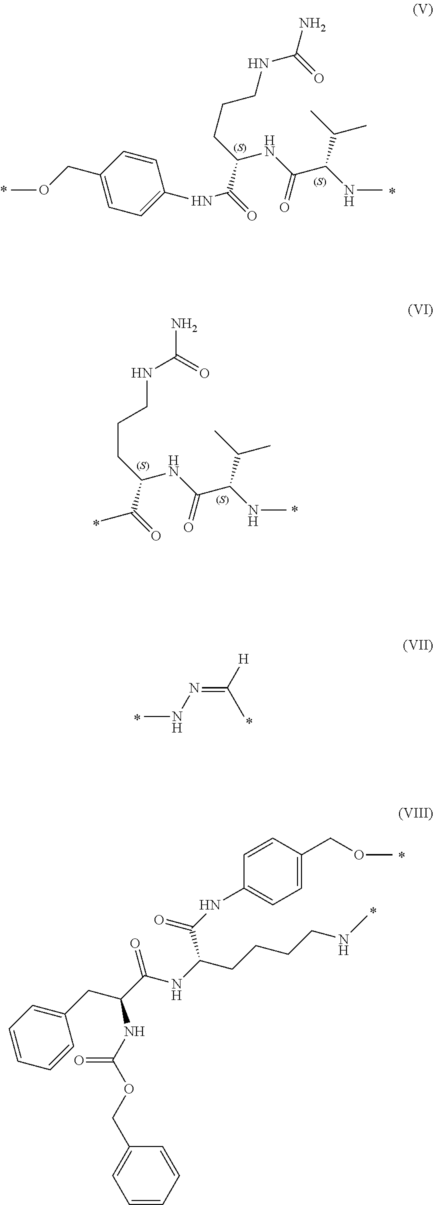

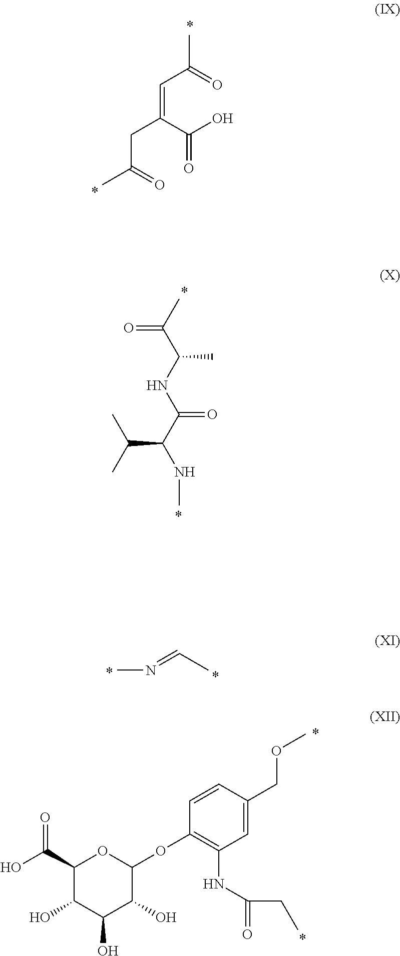

wherein the “*” denotes the attaching points;
wherein the —(C₁-C₃₀)alkylene biradical of X is optionally substituted with one or more radicals selected from the group consisting of —OH, —NRₐR_{b}, —SH, —NHNH₂, —COOR_{c}, —CF₃, —OCF₃, and halogen;
Rₐ, R_{b} and R_{c} are independently selected from the group consisting of H, -phenyl, —(C₁-C₃₀)alkyl, —(C₂-C₃₀)alkenyl, —(C₁-C₃₀)alkylphenyl, and -phenyl(C₁-C₃₀)alkyl;
wherein a and a′ are integers independently ranging from 0 to 1;
r, s, t and u are integers independently ranging from 0 to 500, wherein at least one of r or t are >1;
R₉ and R₁₇ are a radical independently selected from the group consisting of H, —(C₁-C₃₀)alkyl, —(C₂-C₃₀)alkenyl, —(C₁-C₃₀)alkyl-Rⁱ¹, —(C₁-C₃₀)alkyl-O—Rⁱⁱⁱ¹, —(C₁-C₃₀)alkyl-NR^{iv1}R^{v1}, —C(O)—R^{vi1}, —(C₁-C₁₂)alkyl-CO—NH₂, and a radical selected from the group consisting of (XIII), (XIV), (XV), (XVI), (XVII), (XVIII), (XIX), (XX), and (XXI)

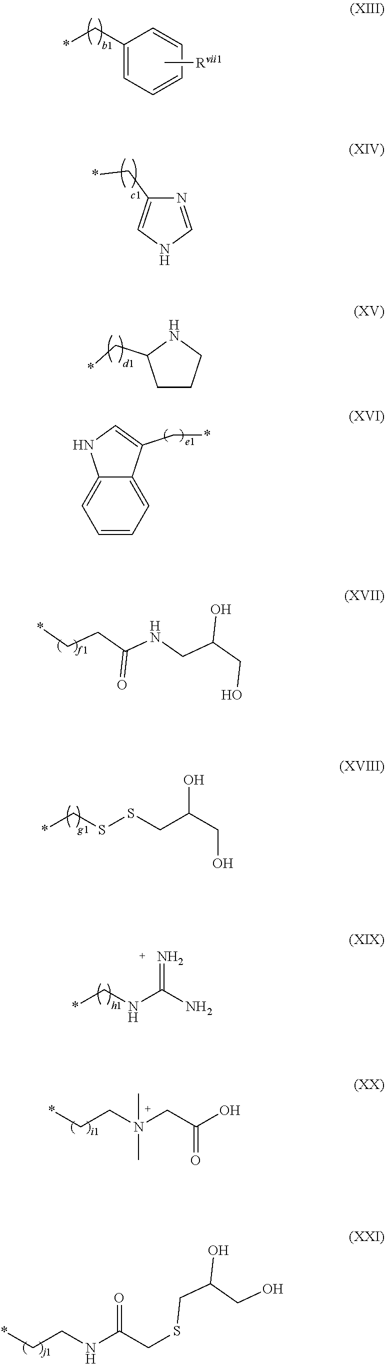

wherein “*” denotes the attaching point;
Rⁱ¹ is selected from the group consisting of H, —(C₁-C₃₀)alkyl, —(C₂-C₃₀)alkenyl, —(C₂-C₃₀)alkynyl, halogen, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NH₂, —N((C₁-C₃₀)alkyl)₂, —NH(C₁-C₃₀)alkyl, —NHC(O)—(C₁-C₃₀)alkyl, —NHC(O)O(C₁-C₃₀)alkyl, —NHC(O)NH₂, —NHC(O)N(CH₃)₂, —NHS(O)₂(C₁-C₃₀)alkyl, —NHSO₂NH₂, —C(O)(C₁-C₃₀)alkyl, —CON((C₁-C₃₀)alkyl)₂; —NO₂, —CN, —OC(O)—(C₁-C₃₀)alkyl, —OC(O)O(C₁-C₃₀)alkyl, —OC(O)NH₂, —OC(O)N((C₁-C₃₀)alkyl)₂, —SeH, —SH, —S(C₁-C₃₀)alkyl, —S(O)H, —S(O)(C₁-C₃₀)alkyl, and —SO₂(C₁-C₃₀)alkyl;
R^{vii1} is selected from the group consisting of H, —(C₁-C₃₀)alkyl, —(C₂-C₃₀)alkenyl, —(C₂-C₃₀)alkynyl, —OAlkyl(C₁-C₁₂), F, Cl, Br, I, —CF₃, —OCF₃, —NO₂, —CN, —NH₂, —(C₁-C₃₀)alkylNH₂, —N((C₁-C₃₀)alkyl)₂, and —NH(C₁-C₃₀)alkyl,
Rⁱⁱ¹, R^{iv1} and R^{v1} are independently selected from the group consisting of H, —(C₁-C₅₀)alkyl, —(C₁-C₅₀)alkylNH₂, —(C₁-C₅₀)alkyl-N((C₁-C₅₀)alkyl)₂, and —(C₁-C₅₀)alkyl-NH(C₁-C₅₀)alkyl;
R^{vi1} is selected from the group consisting of H, —(C₁-C₃₀)alkyl, —(C₂-C₃₀)alkenyl, —(C₁-C₃₀)alkylNH₂, —NH₂, —(C₁-C₃₀)alkyl-N((C₁-C₃₀)alkyl)₂, —NH(C₂-C₃₀)alkenyl, —(C₁-C₃₀)alkyl-NH(C₁-C₃₀)alkyl, —NH-oleic, —NH-noneic, and —NH-lipoic.
   wherein Rⁱ¹, Rⁱ¹, Rⁱⁱ¹, R^{iv1}, R^{v1}, R^{vi1}, and R^{vi1} are optionally substituted with one or more substituents selected from the group consisting of —OH, halogen, —O(C₁-C₃₀)alkyl, —CF₃, —OCF₃, —NH₂, —(C₁-C₃₀)alkyl, —SH, —NHNH₂, —NHCH₃, —N(CH₃)₂, —NCH(CH₃)₂ and —(C₁-C₃₀)alkyl-OH;
b1, c1, d1, e1, f1, g1, h1, i1, and j1 are integers independently ranging from 1 to 20;
wherein W1 and W2 are each independently selected from CH and N;
R₆, R₇, R₁₄, R₁₅ and R₁₉ are independently selected from the group consisting of H, —(C₁-C₃₀)alkyl, —(C₂-C₃₀)alkenyl, —(C₂-C₃₀)alkynyl, —(C₁-C₃₀)alkyl-Rⁱ², —(C₁-C₃₀)alkyl-O—Rⁱⁱ², —(C₁-C₃₀)alkyl-NR^{iv2}R^{v2}, —C(O)—R^{vi2}, and a radical selected from the group consisting of (XXII), (XXIII), (XXIV), (XXV), (XXVI), (XXVII), (XXVIII), (XXIX), (XXX), and (XXXI)

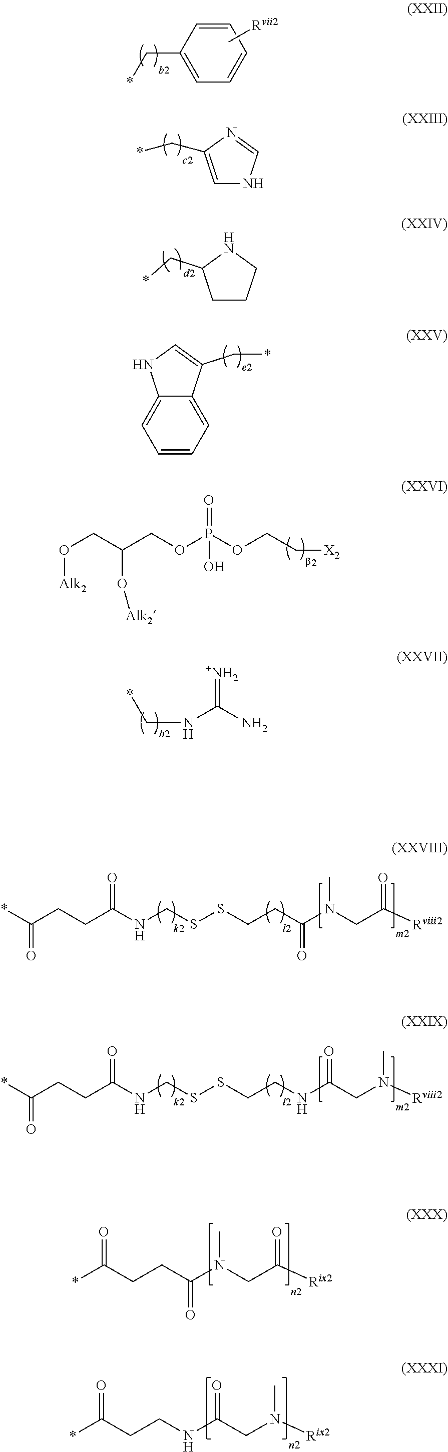

wherein “*” denotes the attaching point;
Rⁱ² is selected from the group consisting of H, —(C₁-C₃₀)alkyl, —(C₂-C₃₀)alkenyl, —(C₂-C₃₀)alkynyl, —OAlkyl(C₁-C₆), halogen, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NO₂, —CN, —OC(O)—(C₁-C₃₀)alkyl, —OC(O)O(C₁-C₃₀)alkyl, —OC(O)NH₂, —OC(O)N((C₁-C₃₀)alkyl)₂, —SH, —S(C₁-C₃₀)alkyl, —S(O)H, —S(O)(C₁-C₃₀)alkyl, —SO₂(C₁-C₃₀)alkyl;
Rⁱⁱⁱ², R^{iv2}, and R^{v2} are independently selected from the group consisting of H, —(C₁-C₃₀)alkyl, —(C₂-C₃₀)alkenyl, —(C₁-C₃₀)alkylNH₂, —(C₁-C₃₀)alkyl-N((C₁-C₃₀)alkyl)₂, —(C₁-C₃₀)alkyl-NH(C₁-C₃₀)alkyl, and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI)

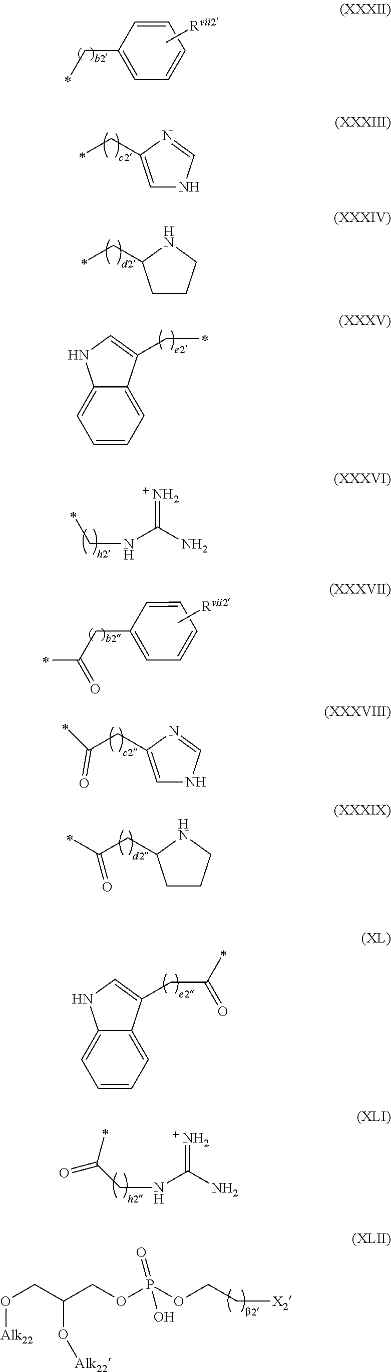

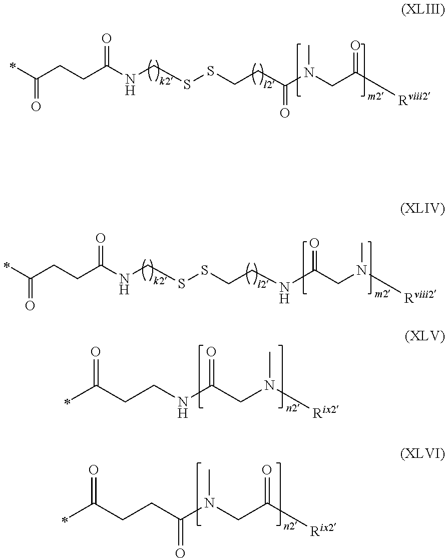

wherein R^{vii2}, R^{ix2}, R^{viii2′}, and R^{ix2″} are independently selected from the group consisting of H, —(C₁-C₁₂)alkyl, —(C₁-C₁₂)alkylNH₂, —(C₁-C₁₂)alkyl-N((C₁-C₁₂)alkyl)₂, —(C₁-C₁₂)alkyl-NH(C₁-C₁₂)alkyl, —O(C₁-C₁₂)alkyl, —COH, —CO(C₁-C₁₂)alkyl, and —O(C₂-C₃₀)alkenyl,
R^{vii2} and R^{vii2′} are independently selected from the group consisting of H, —(C₁-C₃₀)alkyl, —(C₂-C₃₀)alkenyl, —(C₂-C₃₀)alkynyl, —OAlkyl(C₁-C₁₂), F, Cl, Br, I, —CF₃, —OCF₃, —NO₂, —CN, —NH₂, —(C₁-C₃₀)alkylNH₂, —N((C₁-C₃₀)alkyl)₂, and —NH(C₁-C₃₀)alkyl,
R^{vi2} is selected from the group consisting of H, —OH, —(C₁-C₃₀)alkyl, —(C₂-C₃₀)alkenyl, —(C₁-C₃₀)alkyl-COOH, —(C₂-C₃₀)alkenyl-COOH, —(C₁-C₃₀)alkylNH₂, —NH₂, —(C₁-C₃₀)alkyl-N((C₁-C₃₀)alkyl)₂, —O—(C₁-C₃₀)alkyl, —NH(C₂-C₃₀)alkenyl, —(C₁-C₃₀)alkyl-NH(C₁-C₃₀)alkyl, —NH-oleic, —NH-noneic, —NH-lipoic, and —CH═CH(COOH)—CH₂—COOH
wherein Alk₂, Alk₂₂, Alk₂′ and Alk₂₂′ are each independently selected from the group consisting of linear or branched —(C₁-C₃₀)alkyl and linear or branched —(C₂-C₃₀)alkenyl; β₂ and β2′ are each independently an integer from 0 to 6, and X₂ and X₂′ are each independently selected from —NH—, —COO—, and —O—;
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of H, OH, halogen, —O(C₁-C₃₀)alkyl, —CF₃, —OCF₃, —NH₂, —(C₁-C₃₀)alkyl, —(C₂-C₃₀)alkenyl, —(C₂-C₃₀)alkynyl, —SH, —NHNH₂, —NHCH₃, —N(CH₃)₂, —NCH(CH₃)₂ and —(C₁-C₃₀)alkyl-OH;
b2, c2, d2, e2, h2, k2, l2, k2′, l2′, b2″, c2″, d2″, e2″, and h2″ are integers independently ranging from 1 to 20;
b2′, c2′, d2′, e2′, and h2′ are integers independently ranging from 0 to 20;
m2, n2, m2′, and n2′ are integers independently ranging from 1 to 200;
wherein R₃, R₄, R₁₁ and R₁₃ are a biradical independently selected from the group consisting of —(C₁-C₆)alkyl-, —(C₁-C₆)alkyl-S—S—(C₁-C₆)alkyl-, —(C₁-C₆)alkyl-O—(C₁-C₆)alkyl-, and —(C₁-C₆)alkyl-NH—(C₁-C₆)alkyl-;
R₃, R₄, R₁₁ and R₁₃ are optionally substituted by one ore more substituents selected from the group consisting of —NH₂ and —(C₁-C₆)alkyl-NH₂;
with the proviso that R₃ is absent when a=1, and R₁₁ is absent when a′=1;
R₅, R₈, R₁₀, R₁₂, R₁₆ and R₁₈ are a radical independently selected from the group consisting of H and —(C₁-C₆)alkyl.

Clause 2. The compound of formula I according to clause 1, wherein α, α′ and α″ are 0, and the compound of formula I is according to formula Ib

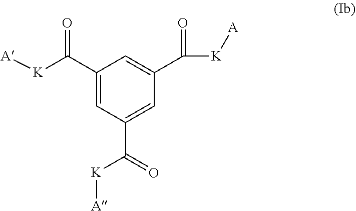

Clause 3. The compound of formula I according to clause 1, wherein α, α′ and α″ are 1, and the compound of formula I is according to formula Ia

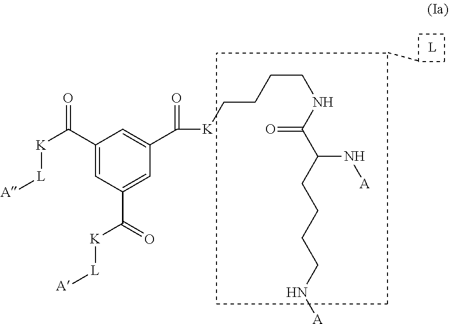

Clause 4. The compound of formula I according to any of clauses 1-3, wherein R¹ is a biradical selected from the group consisting of

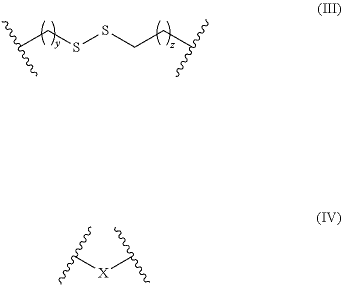

wherein the wavy lines denote the attaching points;
wherein y and z are integers independently ranging from 1 to 6; particularly, y and z are independently 1, 2, 3, 4, 5, or 6;
X is a birradical selected from straight or branched —(C₁-C₁₂)alkylene-, —(C₁-C₆)alkyl-COO—, and a biradical selected from the group consisting of (V), (VI), (VII), (VIII), (IX), (X), (XI) and (XII)
wherein the straight or branched —(C₁-C₁₂)alkylene biradical of X is optionally substituted with one or more radicals selected from the group consisting of —OH, —NRₐR_{b}, —SH, —NHNH₂, —COOR_{c}, —CF₃, —OCF₃, and halogen;
Rₐ, R_{b} and R_{c} are radicals independently selected from the group consisting of H, phenyl, —(C₁-C₁₂)alkyl, —(C₂-C₃₀)alkenyl, —(C₁-C₁₂)alkylphenyl, and -phenyl(C₁-C₁₂)alkyl.

Clause 5. The compound of formula I according to any of clauses 1-4, wherein
R₉ and R₁₇ are a radical independently selected from the group consisting of H, —(C₁-C₁₂)alkyl, —(C₂-C₃₀)alkenyl, —(C₁-C₁₂)alkyl-Rⁱ¹, —(C₁-C₁₂)alkyl-O—Rⁱⁱⁱ¹, —(C₁-C₁₂)alkyl-NR^{iv1}R^{v1}, —C(O)—R^{vi1}, —(C₁-C₁₂)alkyl-CO—NH₂ and a radical selected from the group consisting of (XIII), (XIV), (XV), (XVI), (XVII), (XVIII), (XIX), (XX), and (XXI);
Rⁱ¹ is selected from the group consisting of H, F, Cl, Br, I, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NH₂, —N((C₁-C₁₂)alkyl)₂, —NH(C₁-C₁₂)alkyl, —NHC(O)—(C₁-C₁₂)alkyl, —NHC(O)O(C₁-C₁₂)alkyl, —NHC(O)NH₂, —NHC(O)N(CH₃)₂, —NHS(O)₂(C₁-C₁₂)alkyl, —NHSO₂NH₂, —SH, —S(C₁-C₁₂)alkyl, —S(O)H, —S(O)(C₁-C₁₂)alkyl, —SO₂(C₁-C₁₂)alkyl, —SeH, —C(O)(C₁-C₁₂)alkyl, and —CON((C₁-C₁₂)alkyl)₂;
R^{vii1} is selected from the group consisting of H, methyl, ethyl, propyl, isopropyl, butyl, —(C₂-C₃₀)alkenyl, —(C₂-C₃₀)alkynyl, —OAlkyl(C₁-C₁₂), F, Cl, Br, I, —CF₃, —OCF₃, —NO₂, —CN, —NH₂, —(C₁-C₆)alkylNH₂, —N((C₁-C₆)alkyl)₂, and —NH(C₁-C₆)alkyl;
Rⁱⁱⁱ¹, R^{iv1} and R^{v1} are independently selected from the group consisting of H, —(C₁-C₁₂)alkyl, —(C₁-C₁₂)alkylNH₂, —(C₁-C₁₂)alkyl-N((C₁-C₁₂)alkyl)₂, and —(C₁-C₁₂)alkyl-NH(C₁-C₁₂)alkyl;
R^{vi1} is selected from the group consisting of H, —(C₁-C₁₂)alkyl, —(C₂-C₃₀)alkenyl, —(C₁-C₁₂)alkylNH₂, —NH₂, —(C₁-C₁₂)alkyl-N((C₁-C₁₂)alkyl)₂, —NH(C₂-C₃₀)alkenyl, —(C₁-C₁₂)alkyl-NH(C₁-C₁₂)alkyl, —NH-oleic, —NH-noneic, and —NH-lipoic;
wherein Rⁱ¹, Rⁱⁱ¹, Rⁱⁱⁱ¹, R^{iv1}, R^{v1}, R^{vi1}, and R^{vii1} are optionally substituted with one or more substituents selected from the group consisting of —OH, F, Cl, Br, I, —O(C₁-C₆)alkyl, —CF₃, —OCF₃, —NH₂, —(C₁-C₆)alkyl, —SH, —NHNH₂, —NHCH₃, —N(CH₃)₂, —NCH(CH₃)₂ and —(C₁-C₆)alkyl-OH;
b1, c1, d1, e1, f1, g1, h1, i1, and j1 are integers independently ranging from 1 to 6.

Clause 6. The compound of formula I according to any of clauses 1-5, wherein
R₉ and R₁₇ are a radical independently selected from H, methyl, ethyl, propyl, isopropyl, butyl, n-butyl, —(C₂-C₃₀)alkenyl, —CH₂SCH₃, —CH₂CH₂SCH₃, —CH₂CH₂SCH₂CH₃, —CH₂SCH₂CH₃, —CH₂SH, —CH₂—SeH, —CH₂CH₂SH, —CH₂NH₂, —CH₂CH₂NH₂, —CH₂CH₂CH₂NH₂, —CH₂CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), —C(O)H, —C(O)OCH₃, —C(O)OCH₂CH₃, —C(O)OCH(CH₃)₂, —C(O)CH₂NH₂, —C(O)CH₂CH₂NH₂, —C(O)CH(CH₃)CH₂NH₂, —C(O)CH₂NHCH₃, —C(O)CH₂NHCH₂CH₃, —C(O)CH₂CH₂NHCH₂CH₃, —C(O)CH₂CH₂CH₂NHCH₂CH₃, —C(O)CH₂CH₂CH₂NHCH₂CH₂CH₃, —C(O)CH₂CH(CH₃)CH₂NHCH₃, —C(O)CH₂CH(CH₃)CH₂NHCH₂CH₃, —C(O)CH₂CH₂NHCH₂CH₂NH₂, —C(O)CH₂CH₂CH₂NHCH₂CH₂CH₂NH₂, —C(O)CH₂CH₂N((CH(CH₃)₂)), —C(O)CH₂CH₂CH₂N((CH(CH₃)₂)), —C(O)CH₂CH₂NH(CH(CH₃)₂), —C(O)CH₂CH₂CH₂NH(CH(CH₃)₂), —CH₂COOCH₃, —CH₂CH₂COOCH₃, —CH₂COOCH₂CH₃, —CH₂CH₂COOCH₂CH₃, —CH₂COOCH(CH₃)₂, —CH₂CONH₂, —CH₂CH₂CONH₂, —CH₂CH₂CH₂CONH₂, —CONH-oleic, —CONH-noneic, —CONH-lipoic, and a radical selected from the group consisting of (XIII), (XIV), (XV), (XVI), (XVII), (XVIII), (XIX), (XX), and (XXI)
wherein R^{vii1} is selected from the group consisting of H, methyl, ethyl, propyl, isopropyl, butyl, —(C₂-C₃₀)alkenyl, —(C₂-C₃₀)alkynyl, —OAlkyl(C₁-C₁₂), F, Cl, Br, I, —CF₃, —OCF₃, —NO₂, —CN, —NH₂, —(C₁-C₆)alkylNH₂, —N((C₁-C₆)alkyl)₂, and —NH(C₁-C₆)alkyl;
and wherein b1, c1, d1, e1, f1, g1, h1, i1, and i1 are integers independently ranging from 1 to 6.

Clause 7. The compound of formula I according to any of clauses 1-6, wherein
R₉ and R₁₇ are a radical independently selected from the group consisting of H, methyl, ethyl, propyl, isopropyl, butyl, n-butyl, —CH₂SCH₃, —CH₂CH₂SCH₃, —CH₂CH₂SCH₂CH₃, —CH₂SCH₂CH₃, —CH₂SH, —CH₂CH₂SH, —CH₂SeH, —CH₂NH₂, —CH₂CH₂NH₂, —CH₂CH₂C H₂NH₂, —CH₂CH₂CH₂CH₂NH₂, and a radical selected from the group consisting of (XIII), (XIV), (XV), (XVI), (XVII), (XVIII), (XIX), (XX), and (XXI);
wherein R^{vii1} is selected from H, methyl, ethyl, propyl, isopropyl, butyl, —(C₂-C₃₀)alkenyl, —(C₂-C₃₀)alkynyl, —OAlkyl(C₁-C₆), F, Cl, Br, I, —CF₃, —OCF₃, —NO₂, —CN, —NH₂, —CH₂NH₂, —CH₂CH₂NH₂, —CH(CH₃)NH₂, —CH₂CH(CH₃)NH₂, —CH₂CH₂CH₂NH₂, —N(CH₃)₂, —N(CH₂CH₃)₂, —NCH(CH₃)₂, —NHCH₃, —NHCH₂CH₃, and —NHCH(CH₃)₂;
and wherein b1, c1, d1, e1, f1, g1, h1, i1, and j1 are integers independently ranging from 1 to 4.

Clause 8. The compound of formula I according to any of clauses 1-7, wherein
R₆, R₇, R₁₄, R₁₅ and R₁₉ are independently selected from the group consisting of H, —(C₁-C₃₀)alkyl, —(C₂-C₃₀)alkenyl, —(C₂-C₃₀)alkynyl, —(C₁-C₃₀)alkyl-Rⁱ², —(C₁-C₃₀)alkyl-O—Rⁱⁱⁱ², —(C₁-C₃₀)alkyl-NR^{iv2}R^{v2}, —C(O)—R^{vi2}, and a radical selected from the group consisting of (XXII), (XXIII), (XXIV), (XXV), (XXVI), (XXVII), (XXVIII), (XXIX), (XXX), and (XXXI)
Rⁱ² is selected from the group consisting of H, —(C₁-C₁₂)alkyl, —OAlkyl(C₁-C₆), F, Cl, Br, I, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NO₂, —CN, —OC(O)—(C₁-C₁₂)alkyl, —OC(O)O(C₁-C₁₂)alkyl, —OC(O)NH₂, —OC(O)N((C₁-C₁₂)alkyl)₂, —SH, —S(C₁-C₁₂)alkyl, —S(O)H, —S(O)(C₁-C₁₂)alkyl, —SO₂(C₁-C₁₂)alkyl;
Rⁱⁱⁱ², R^{iv2}, and R^{v2} are independently selected from the group consisting of H, —(C₁-C₁₂)alkyl, —(C₂-C₃₀)alkenyl, —(C₁-C₃₀)alkylNH₂, —(C₁-C₁₂)alkyl-N((C₁-C₁₂)alkyl)₂, —(C₁-C₁₂)alkyl-NH(C₁-C₁₂)alkyl, and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI)
wherein R^{viii2}, R^{ix2}, R^{viii2′}, and R^{ix2″} are independently selected from the group consisting of H, —(C₁-C₆)alkyl, —(C₁-C₆)alkylNH₂, —(C₁-C₆)alkyl-N((C₁-C₆)alkyl)₂, —(C₁-C₆)alkyl-NH(C₁-C₆)alkyl, —O(C₁-C₆)alkyl, —COH, —CO(C₁-C₆)alkyl, and —O(C₂-C₁₂)alkenyl,
R^{vii2} and R^{vii2′} are independently selected from the group consisting of H, —(C₁-C₁₂)alkyl, —(C₂-C₃₀)alkenyl, —(C₂-C₃₀)alkynyl, —O(C₁-C₆)Alkyl, F, Cl, Br, I, —CF₃, —OCF₃, —NO₂, —CN, —NH₂, —(C₁-C₁₂)alkylNH₂, —N((C₁-C₁₂)alkyl)₂, and —NH(C₁-C₁₂)alkyl;
R^{vi2} is selected from the group consisting of H, —OH, —(C₁-C₁)alkyl, —(C₂-C₃₀)alkenyl, —(C₁-C₁₂)alkyl-COOH, —(C₂-C₃₀)alkenyl-COOH, —(C₁-C₁₂)alkylNH₂, —(C₁-C₁₂)alkyl-N((C₁-C₁₂)alkyl)₂, —O—(C₁-C₁₂)alkyl, —NH(C₂-C₁₂)alkenyl, —(C₁-C₁₂)alkyl-NH(C₁-C₁₂)alkyl, —NH-oleic, —NH-noneic, —NH-lipoic, and —CH═CH(COOH)—CH₂—COOH
wherein Alk₂, Alk₂₂, Alk₂′ and Alk₂₂′ are each independently selected from the group consisting of linear or branched —(C₁-C₁₂)alkyl and linear or branched —(C₂-C₃₀)alkenyl; β₂ and β₂′ are each independently an integer from 0 to 6, and X₂ and X₂′ are each independently selected from the group consisting of —NH—, —COO—, and —O—;
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of OH, F, Cl, Br, I, —O(C₁-C₆)alkyl, —CF₃, —OCF₃, —NH₂, —(C₁-C₆)alkyl-NHNH₂, —NHCH₃, —N(CH₃)₂, —NCH(CH₃)₂ and —(C₁-C₆)alkyl-OH;
b2, c2, d2, e2, h2, k2, l2, k2′, l2′, b2″, c2″, d2″, e2″, and h2″ are integers independently ranging from 1 to 6;
b2′, c2′, d2′, e2′, and h2′ are integers independently ranging from 0 to 6;
m2, n2, m2′, and n2′ are integers independently ranging from 1 to 150.

Clause 9. The compound of formula I according to any of clauses 1-8, wherein
R₆, R₇, R₁₄, R₁₅ and R₁₉ are independently selected from the group consisting of H, methyl, ethyl, propyl, isopropyl, butyl, —CH₂OCH₃, —CH₂CH₂OCH₃, —CH₂CH₂OCH₂CH₃, —CH₂CH₂OCH(CH₃)₂, —CH₂OCH₂CH₃, —CH₂OCH(CH₃)₂, —(C₂-C₃₀)alkenyl, —(C₂-C₃₀)alkynyl, —CH₂SCH₃, —CH₂CH₂SCH₃, —CH₂CH₂SCH₂CH₃, —CH₂SCH₂CH₃, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), —C(O)H, —C(O)OCH₃, —C(O)OCH₂CH₃, —C(O)OCH(CH₃)₂, —C(O)CH₂NH₂, —C(O)CH₂CH₂NH₂, —C(O)CH(CH₃)CH₂NH₂, —C(O)CH₂NHCH₃, —C(O)CH₂NHCH₂CH₃, —C(O)CH₂CH₂NHCH₂CH₃, —C(O)CH₂CH₂CH₂NHCH₂CH₃, —C(O)CH₂CH₂CH₂NHCH₂CH₂CH₃, —C(O)CH₂CH(CH₃)CH₂NHCH₃, —C(O)CH₂CH(CH₃)CH₂NHCH₂CH₃, —C(O)CH₂CH₂NHCH₂CH₂NH₂, —C(O)CH₂CH₂CH₂NHCH₂CH₂CH₂NH₂, —C(O)CH₂CH₂N((CH(CH₃)₂)), —C(O)CH₂CH₂CH₂N((CH(CH₃)₂)), —C(O)CH₂CH₂NH(CH(CH₃)₂), —C(O)CH₂CH₂CH₂NH(CH(CH₃)₂), —CH₂COOH, —CH₂CH₂COOH, —CH₂COOCH₃, —CH₂CH₂COOCH₃, —CH₂COOCH₂CH₃, —CH₂CH₂COOCH₂CH₃, —CH₂COOCH(CH₃)₂, —(C₁-C₆)alkyl-Rⁱ², —(C₁-C₆)alkyl-O—Rⁱⁱⁱ², —(C₁-C₆)alkyl-NR^{iv2}R^{v2}, —CONH-oleic, —CONH-noneic, —CONH-lipoic, and a radical selected from the group consisting of (XXII), (XXIII), (XXIV), (XXV), (XXVI), (XXVII), (XXVIII), (XXIX), (XXX), and (XXXI)
Rⁱ² is selected from the group consisting of imidazole, N-methylpyrrole, pyrrole, pyrrolidine, pyrane, pyridine, piperidine, pyrimidine, —OC(O)NH₂, —OC(O)N((C₁-C₆)alkyl)₂;
Rⁱⁱⁱ², R^{iv2}, and R^{v2} are independently selected from the group consisting of —(C₂-C₃₀)alkenyl, —(C₂-C₃₀)alkynyl, —(C₁-C₆)alkyl-NH₂, —(C₁-C₆)alkyl-N((C₁-C₆)alkyl)₂, —(C₁-C₆)alkyl-NH(C₁-C₆)alkyl, and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI)
R^{vii2} and R^{vii2′} are independently selected from H, methyl, ethyl, propyl, isopropyl, butyl, —OAlkyl(C₁-C₆), F, Cl, Br, I, —CF₃, —OCF₃, —NO₂, —CN, —NH₂, —(C₁-C₆)alkyl-NH₂, —N((C₁-C₆)alkyl)₂, —NH(C₁-C₆)alkyl;
wherein R^{viii2}, R^{ix2}, R^{viii2′}, and R^{ix2″} are independently selected from H, —(C₁-C₆)alkyl, —(C₂-C₆)alkenyl, —(C₂-C₆)alkynyl, —(C₁-C₆)alkylNH₂, —(C₁-C₆)alkyl-N((C₁-C₆)alkyl)₂, —(C₁-C₆)alkyl-NH(C₁-C₆)alkyl;
wherein Rⁱ², Rⁱⁱⁱ², R^{iv2}, R^{v2}, R^{vi2}, R^{vii2}, R^{viii2}, R^{ix2}, R^{viii′}, and R^{ix2′} are optionally substituted by one or more substituents selected from the group consisting of OH, F, Cl, Br, —OCH₃, —OCH₂CH₃, —OCH(CH₃)₂, —CF₃, —OCF₃, —NH₂, —CH₃, —CH₂CH₃, —CH(CH₃)₂, —CH₂OH, —CH₂CH₂OH, and —CH₂CH(OH)CH₃;
b2, c2, d2, e2, h2, k2, l2, k2′, l2′, b2″, c2″, d2″, e2″, and h2″ are integers independently ranging from 1 to 4;
b2′, c2′, d2′, e2′, and h2′ are integers independently ranging from 0 to 4;
m2, n2, m2′, and n2′ are integers independently ranging from 1 to 120.

Clause 10. The compound of formula I according to any of claims 1-9, wherein R₁ is a biradical selected from the group consisting of —CH₂CH₂—S—S—CH₂CH₂—, —CH₂CH₂CH₂—S—S—CH₂CH₂CH₂—, —CH₂—, —CH₂CH₂—, —CH₂CH(CH₃)CH₂—, —CH₂CH(CH₂CH₃)CH₂—, —CH₂CH₂CH₂CH₂—, —CH₂COO—, —CH₂CH₂COO—, —CH₂CHCH₃COO—, —CH₂CH₂CH₃CH₂COO—, and a biradical selected from the group consisting of (V), (VI), (VII), (VIII), (IX), (X), (XI) and (XII);
R₉ and R₁₇ are a radical independently selected from the group consisting of H, methyl, ethyl, propyl, isopropyl, butyl, n-butyl, —CH₂SCH₃, —CH₂CH₂SCH₃, —CH₂CH₂SCH₂CH₃, —CH₂SCH₂CH₃, —CH₂SH, —CH₂CH₂SH, —CH₂SeH, —CH₂NH₂, —CH₂CH₂NH₂, —CH₂CH₂CH₂NH₂, —CH₂CH₂CH₂CH₂NH₂, and a radical selected from the group consisting of (XIII), (XIV), (XV), (XVI), (XVII), (XVIII), (XIX), (XX), and (XXI);
wherein R^{vii1} is selected from H, methyl, ethyl, propyl, isopropyl, butyl, —(C₂-C₃₀)alkenyl, —(C₂-C₃₀)alkynyl, —OCH₃, —OCH₂CH₃, F, Cl, Br, I, —CF₃, —OCF₃, —NO₂, —CN, —NH₂, —CH₂NH₂, —CH₂CH₂NH₂, —CH(CH₃)NH₂, —CH₂CH(CH₃)NH₂, —CH₂CH₂CH₂NH₂, —N(CH₃)₂, —N(CH₂CH₃)₂, —NCH(CH₃)₂, —NHCH₃, —NHCH₂CH₃, and —NHCH(CH₃)₂;
wherein b1, c1, d1, e1, f1, g1, h1, i1, and j1 are integers independently ranging from 1 to 6;
wherein r, s, t and u are integers independently ranging from 0 to 250, wherein at least one of r or t is ≥1.

Clause 11. The compound of formula I according to any of claims 1-10, wherein
R¹ is a biradical selected from the group consisting of —CH₂CH₂—S—S—CH₂CH₂—, —CH₂CH₂CH₂—S—S—CH₂CH₂CH₂—, —CH₂—, —CH₂CH₂—, —CH₂CH(CH₃)CH₂—, —CH₂CH(CH₂CH₃)CH₂—, —CH₂CH₂CH₂CH₂—, —CH₂COO—, —CH₂CH₂COO—, —CH₂CHCH₃COO—, —CH₂CH₂CH₃CH₂COO—, and a biradical selected from the group consisting of (V), (VI), (VII), (VIII), (IX), (X), (XI) and (XII)

R₉ and R₁₇ are a radical independently selected from H, methyl, ethyl, propyl, isopropyl, butyl, n-butyl, —CH₂SCH₃, —CH₂CH₂SCH₃, —CH₂CH₂SCH₂CH₃, —CH₂SCH₂CH₃, —CH₂SH, —CH₂CH₂SH, —CH₂SeH, —CH₂NH₂, —CH₂CH₂NH₂, —CH₂CH₂CH₂NH₂, —CH₂CH₂CH₂CH₂NH₂, and a radical selected from the group consisting of (XIII), (XIV), (XV), (XVI), (XVII), (XVIII), (XIX), (XX), and (XXI)
R^{vii1} is selected from H, methyl, ethyl, propyl, isopropyl, butyl, —OCH₃, —OCH₂CH₃, F, Cl, Br, I, —CF₃, —OCF₃, —NO₂, —CN, —NH₂, —CH₂NH₂, —CH₂CH₂NH₂, —CH₂CH(CH₃)NH₂, —CH₂CH₂CH₂NH₂, —N(CH₃)₂, —N(CH₂CH₃)₂, —NCH(CH₃)₂, —NHCH₃, —NHCH₂CH₃, and —NHCH(CH₃)₂;
b1, c1, d1, e1, f1, g1, h1, i1, and j1 are integers independently ranging from 1 to 6;

R₆, R₇, R₁₄, R₁₅ and R₁₉ are independently selected from H, methyl, ethyl, propyl, isopropyl, butyl, —CH₂OCH₃, —CH₂CH₂OCH₃, —CH₂CH₂OCH₂CH₃, —CH₂CH₂OCH(CH₃)₂, —CH₂OCH₂CH₃, —CH₂OCH(CH₃)₂, —(C₂-C₃₀)alkenyl, —(C₂-C₃₀)alkynyl, —CH₂SCH₃, —CH₂CH₂SCH₃, —CH₂SH, —CH₂SeH—CH₂CH₂SH, —CH₂CH₂SCH₂CH₃, —CH₂SCH₂CH₃, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), —C(O)H, —C(O)OCH₃, —C(O)OCH₂CH₃, —C(O)OCH(CH₃)₂, —C(O)CH₂NH₂, —C(O)CH₂CH₂NH₂, —C(O)CH(CH₃)CH₂NH₂, —C(O)CH₂NHCH₃, —C(O)CH₂NHCH₂CH₃, —C(O)CH₂CH₂NHCH₂CH₃, —C(O)CH₂CH₂CH₂NHCH₂CH₃, —C(O)CH₂CH₂CH₂NHCH₂CH₂CH₃, —C(O)CH₂CH(CH₃)CH₂NHCH₃, —C(O)CH₂CH(CH₃)CH₂NHCH₂CH₃, —C(O)CH₂CH₂NHCH₂CH₂NH₂, —C(O)CH₂CH₂CH₂NHCH₂CH₂CH₂NH₂, —C(O)CH₂CH₂N((CH(CH₃)₂)), —C(O)CH₂CH₂CH₂N((CH(CH₃)₂)), —C(O)CH₂CH₂NH(CH(CH₃)₂), —C(O)CH₂CH₂CH₂NH(CH(CH₃)₂), —CH₂COOH, —CH₂CH₂COOH, —CH₂COOCH₃, —CH₂CH₂COOCH₃, —CH₂COOCH₂CH₃, —CH₂CH₂COOCH₂CH₃, —CH₂COOCH(CH₃)₂, —(C₁-C₆)alkyl-Rⁱ², —(C₁-C₆)alkyl-O—Rⁱⁱⁱ², —(C₁-C₆)alkyl-NR^{iv2}R^{v2}, —CONH-oleic, —CONH-noneic, —CONH-lipoic, and a radical selected from the group consisting of (XXII), (XXIII), (XXIV), (XXV), (XXVI), (XXVII), (XXVIII), (XXIX), (XXX), and (XXXI)

Rⁱ² is selected from the group consisting of imidazole, N-methylpyrrole, pyrrole, pyrrolidine, pyrane, pyridine, piperidine, pyrimidine, —OC(O)NH₂, —OC(O)N((C₁-C₆)alkyl)₂;
Rⁱⁱⁱ², R^{iv2}, and R^{v2} are independently selected from H, —(C₂-C₃₀)alkenyl, —(C₂-C₃₀)alkynyl, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI);
wherein R^{vii2} and R^{vii2′} are independently selected from H, methyl, ethyl, propyl, isopropyl, butyl, F, Cl, Br, I, —CF₃, —OCF₃, —NO₂, —CN, —NH₂, and —(C₁-C₆)alkyl-NH₂;
wherein R^{viii2}, R^{ix2}, R^{viii2′}, and R^{ix2″} are independently selected from H, methyl, ethyl, propyl, isopropyl, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂);
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of OH, F, Cl, Br, —OCH₃, —OCH₂CH₃, —OCH(CH₃)₂, —CF₃, —OCF₃, —NH₂, —CH₃, —CH₂CH₃, —CH(CH₃)₂, —CH₂OH, —CH₂CH₂OH, and —CH₂CH(OH)CH₃;
b2, c2, d2, e2, h2, k2, l2, k2′, l2′, b2″, c2″, d2″, e2″, and h2″ are integers independently ranging from 1 to 4;
b2′, c2′, d2′, e2′, and h2′ are integers independently ranging from 0 to 4;
m2, n2, m2′, and n2′ are integers independently ranging from 1 to 120.

Clause 12. The compound of formula I according to any of clauses 1-11, which is selected from

| | |
|---|---|
| CP5 | St-PAspDET(100) |
| CP6 | St-PAspDET(50) |
| CP7 | St-S-S-PAspDET(35) |
| CP8 | St-S-S-PAspDET(50) |
| CP9 | St-S-S-PAspDET(100) |
| CP10 | St-PAspDET(71)/DIIPA(29) |
| CP11 | St-S-S-PAspDET(45)/DIIPA(21)) |
| CP12 | St-S-S-PAspDET(78)/DIIPA(21) |
| CP13 | St-S-S-PAspDET(91)/DIIPA(29) |
| CP14 | St-S-S-PAspDET(73)/DIIPA(34) |
| CP17 | St-S-S-PAspDET(47)-co-PLeu(9) |
| CP18 | St-S-S-PAspDET(54)-co-PPhe(4) |
| CP19 | St-S-S-PAspDET(57)-co-PPhe(5) |
| CP20 | St-S-S-PAspDET(37)-co-PPhe(9) |
| CP21 | St-S-S-PAspDET(48)/DIIPA(18)-co-PLeu(4) |
| CP22 | St-S-S-PAspDET(31)/DIIPA(12)-co-PLeu(7) |
| CP23 | St-S-S-PAspDET(41)/DIIPA(10)-co-PPhe(5) |
| CP30 | St-S-S-PAspDET(42)-b-Psar(37) |
| CP31 | St-S-S-PAspDET(41)-b-Psar(22) |
| CP32 | St-S-S-PAspDET(46)-co-PLeu(8)-b-PSar(50) |
| CP33 | St-S-S-PAspDET51-co-PPhe5-b-PSar44 |
| CP34 | St-S-S-PAspDET48-co-PPhe9-b-PSar36 |
| CP35 | St-S-S-PAspDET(51)/DIIPA(20)-b-PSar(58) |
| CP36 | St-S-S-PAspDET(38)/DIIPA(21)-co-PLeu(11)-b-PSar(50) |
| CP37 | St-S-S-PAspDET(46)/DIIPA(13)-co-PPhe(11)-b-PSar(28) |
| CP38 | St-S-S-PAspDET(100)-graft into-[Succ-PSar(47)-Galactosamine]3 |
| CP39 | St-S-S-PAspDET(91)/DIIPA(29)-graft into-[Succ-PSar(23)-iPr]6 |
| CP40 | St-S-S-PAspDET(100)-graft into-[Succ-ethyl-S-S-ethyl- |
| | PSar(28)]3-COCH₃ |
| CP41 | St-S-S-PAspDET(100)-graft into-[Succ-ethyl-S-S-ethyl- |
| | PSar(28)]2-COCH₃ |
| CP42 | St-S-S-PAspDET(100)-graft into-[Succ-ethyl-S-S-ethyl- |
| | PSar(28)]1-COCH₃ |
| CP43 | St-S-S-PAspDET(100)-graft into-[Succ-ethyl-S-S-ethyl- |
| | PSar(28)]6-COCH₃ |
| CP44 | St-S-S-PAspDET(57)-co-PPhe(5)-graft into-[Succ-ethyl- |
| | S-S-ethyl-PSar(28)]2-COCH₃ |
| CP45 | St-S-S-PAspTEP(100) |
| CP46 | St-S-S-PAspImidazolamine(100) |
| CP48 | St-S-S-PAspDET(49)/Imidazolamine(17) |
| CP49 | St-S-S-PAspDET(86)/Imidazolamine(31) |
| CP50 | St-AlaVal-PAspDET(87)/PPhe(10) |
| CP51 | St-S-S-PAspDET(36)/Imidazolamine(12)-co-PPhe(5) |
| CP52 | St-S-S-PAspDET(72)-graft into-lipoic acid(5) |
| CP53 | St-S-S-PAspDET(65)/Imidazolamine(26) graft into-oleic acid(5) |
| CP54 | St-S-S-PAspDET(65)/Imidazolamine(26) graft into-oleic |
| | acid(5)/nonanoic acid(13) |
| CP55 | St-[Lys(3)]PAspDET(100) |
| CP58 | St-S-S-PAspDET(100)-b-PPro(10) |
| CP59 | St-S-S-PAspDET(119)-b-PPro(14) |

wherein the numerical values mentioned in parenthesis refer to the polymerization degree for each monomeric unit and wherein each polymerization degree value is subject to a reasonable uncertainty which is within the DP range ±20%.

1. A compound of formula I, a pharmaceutically acceptable salt thereof, or any stereoisomer or mixtures of stereoisomers, either of the compound of formula (I) or of any of its pharmaceutically acceptable salts, comprising homo-polypeptides or random or block or graft co-polypeptides: 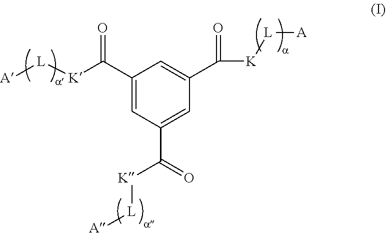
wherein A, A′ and A″ are each independently selected from a radical of formula II; and each of A, A′ and A″ sub-units may be same or different; 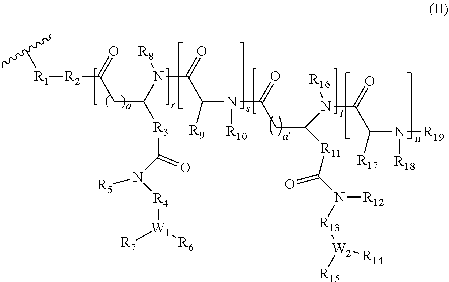
wherein the wavy line denotes the attaching point;
and though the repeating units defined by square brackets with their numerical value, r, s, t and u respectively in the formula II are shown in a particular order for convenience of description, the repeating units may be present in any order and the repeating units may be block or randomly present; and wherein each of the repeating units, may comprise blocks of monomer units which may be the same or different from each other;
wherein K, K′ and K″ are each independently selected from —O— and —NH—;
L is selected from 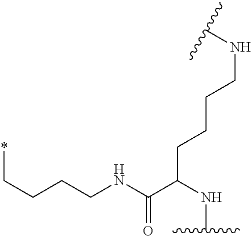 [SMILES: *CCCCNC(=O)C(CCCCN*)N*]
wherein α, α′ and α″ are an integer from 0 to 1; each wavy lines denote the attaching points to A, A′ or A″; and “*” denotes the attaching point to K, K′ or K″;
wherein R₂ is selected from —O— and —NH—
wherein R₁ is a biradical selected from the group consisting of (III) and (IV): 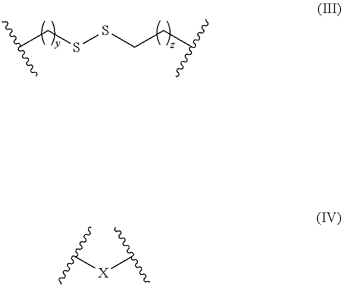
wherein the wavy lines denote the attaching points;
wherein y and z are integers independently ranging from 1 to 20;
X is a biradical selected from the group consisting of —NH—, —NH(C₁-C₆)alkyl-, —O—, —(C₁-C₆)alkyl-COO—, a straight or branched —(C₁-C₃M)alkylene-, and a biradical selected from the group consisting of (V), (VI), (VII), (VIII), (IX), (X), (XI) and (XII): 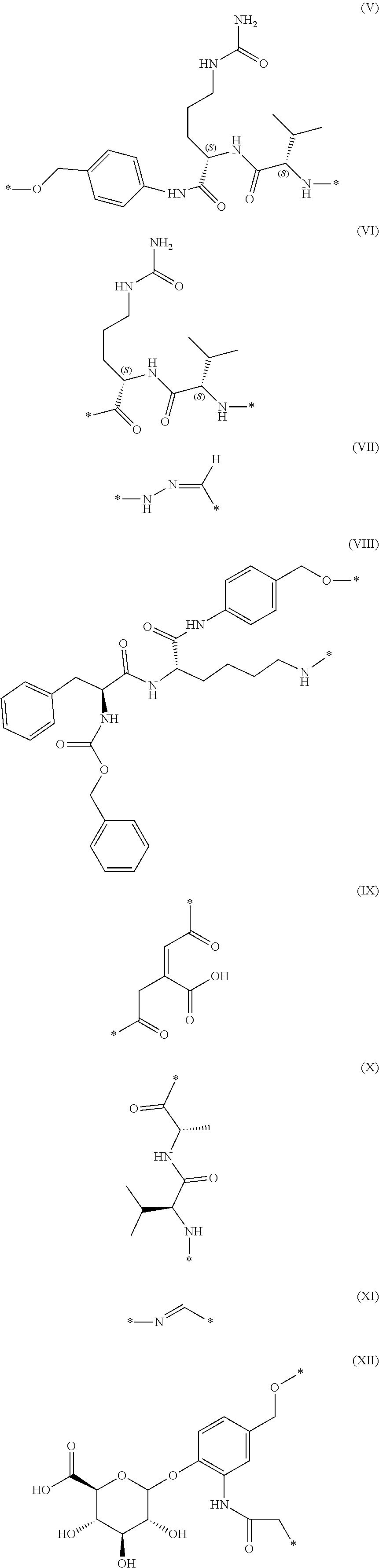
wherein the “*” denotes the attaching points;
wherein the —(C₁-C₃₀)alkylene biradical of X is optionally substituted with one or more radicals selected from the group consisting of —OH, —NRₐR_{b}, —SH, —NHNH₂, —COOR_{c}, —CF₃, —OCF₃, and halogen;
Rₐ, R_{b} and R_{c} are independently selected from the group consisting of H, -phenyl, —(C₁-C₃₀)alkyl, —(C₂-C₃₀)alkenyl, —(C₁-C₃₀)alkylphenyl, and -phenyl(C₁-C₃₀)alkyl;
wherein a and a′ are integers independently ranging from 0 to 1;
r, s, t and u are integers independently ranging from 0 to 500, wherein at least one of r or t are ≥1;
where in the radical of formula (II), the repetitive unit defined by square brackets with the numerical value r is denoted as PAA1; the repetitive unit defined by square brackets with the numerical value s is denoted as PAA2; the repetitive unit defined by square brackets with the numerical value t is denoted as PAA3; and the repetitive unit defined by square brackets with the numerical value u is denoted as PAA4;
wherein the molar ratio of the PAA1 monomer to the PAA2 is from 100/0 to 60/40;
wherein the molar ratio of the PAA1 monomer to the PAA4 is from 100/0 to 60/40;
wherein the molar ratio of the PAA3 monomer to the PAA4 is from 100/0 to 60/40;
wherein the molar ratio of the PAA3 monomer to the PAA2 is from 100/0 to 60/40;
and wherein the molar ratio of the sum of PAA1+PAA3 monomers to the sum of the PAA2+PAA4 is from 100/0 to 60/40;
R₉ and R₁₇ are a radical independently selected from the group consisting of H, —(C₁-C₃₀)alkyl, —(C₂-C₃₀)alkenyl, —(C₁-C₃₀)alkyl-Rⁱ¹, —(C₁-C₃₀)alkyl-COORⁱⁱ¹, —(C₁-C₃₀)alkyl-O—Rⁱⁱⁱ¹, —(C₁-C₃₀)alkyl-NR^{iv1}R^{v1}, —C(O)—R^{vi1}, —(C₁-C₁₂)alkyl-CO—NH₂, and a radical selected from the group consisting of (XIII), (XIV), (XV), (XVI), (XVII), (XVIII), (XIX), (XX), and (XXI): 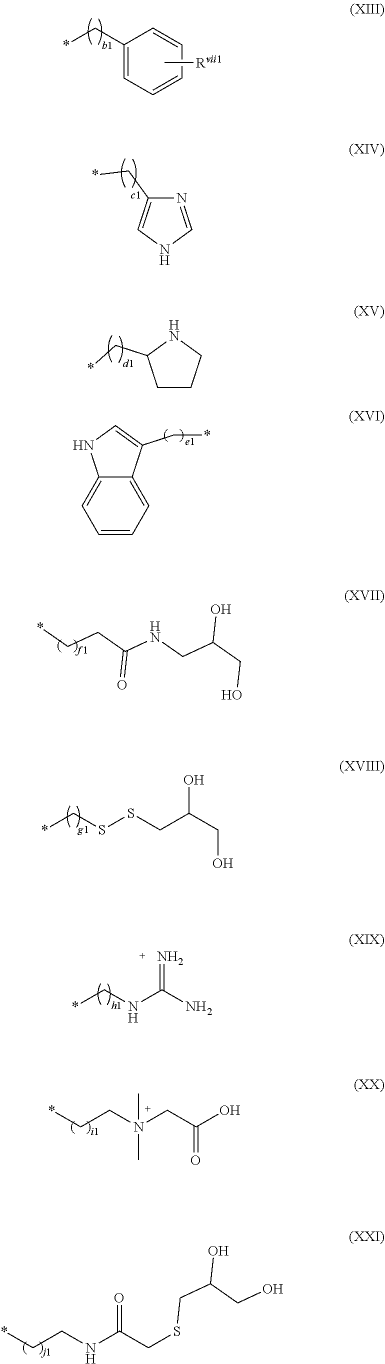
wherein “*” denotes the attaching point;
Rⁱ¹ is selected from the group consisting of H, —(C₁-C₃₀)alkyl, —(C₂-C₃₀)alkenyl, —(C₂-C₃₀)alkynyl, halogen, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NH₂, —N((C₁-C₃₀)alkyl)₂, —NH(C₁-C₃₀)alkyl, —NHC(O)—(C₁-C₃₀)alkyl, —NHC(O)O(C₁-C₃₀)alkyl, —NHC(O)NH₂, —NHC(O)N(CH₃)₂, —NHS(O)₂(C₁-C₃₀)alkyl, —NHSO₂NH₂, —C(O)(C₁-C₃₀)alkyl, —CON((C₁-C₃₀)alkyl)₂; —NO₂, —CN, —O C(O)—(C₁-C₃₀)alkyl, —OC(O)O(C₁-C₃₀)alkyl, —OC(O)NH₂, —OC(O)N((C₁-C₃₀)alkyl)₂, —SeH, —SH, —S(C₁-C₃₀)alkyl, —S(O)H, —S(O)(C₁-C₃₀)alkyl, and —SO₂(C₁-C₃₀)alkyl;
R^{vii1} is selected from the group consisting of H, —(C₁-C₃₀)alkyl, —(C₂-C₃₀)alkenyl, —(C₂-C₃₀)alkynyl, —OAlkyl(C₁-C₁₂), F, Cl, Br, I, —CF₃, —OCF₃, —NO₂, —CN, —NH₂, —(C₁-C₃₀)alkylNH₂, —N((C₁-C₃₀)alkyl)₂, and —NH(C₁-C₃₀)alkyl, Rⁱⁱ¹, Rⁱⁱⁱ¹, R^{iv1} and R^{v1} are independently selected from the group consisting of H, —OH, —(C₁-C₃₀)alkyl, —(C₁-C₃₀)alkylNH₂, —(C₁-C₃₀)alkyl-N((C₁-C₃₀)alkyl)₂, and —(C₁-C₃₀)alkyl-NH(C₁-C₃₀)alkyl;
R^{vi1} is selected from the group consisting of H, —OH, —(C₁-C₃₀)alkyl, —(C₂-C₃₀)alkenyl, —(C₁-C₃₀)alkylNH₂, —NH₂, —(C₁-C₃₀)alkyl-N((C₁-C₃₀)alkyl)₂, —NH(C₂-C₃₀)alkenyl, —(C₁-C₃₀)alkyl-NH(C₁-C₃₀)alkyl, —NH-oleic, —NH-noneic, and —NH-lipoic;
wherein Rⁱ¹, Rⁱⁱ¹, Rⁱⁱⁱ¹, R^{iv1}, R^{v1}, R^{vi1}, and R^{vii1} are optionally substituted with one or more substituents selected from the group consisting of —OH, halogen, —O(C₁-C₃₀)alkyl, —CF₃, —OCF₃, —NH₂, —(C₁-C₃₀)alkyl, —SH, —NHNH₂, —NHCH₃, —N(CH₃)₂, —NCH(CH₃)₂ and —(C₁-C₃₀)alkyl-OH;
b1, c1, d1, e1, f1, g1, h1, i1, and j1 are integers independently ranging from 1 to 20;
wherein W1 and W2 are each independently selected from CH and N;
R₆, R₇, R₁₄, R₁₅ and R₁₉ are independently selected from the group consisting of H, —(C₁-C₃₀)alkyl, —(C₂-C₃₀)alkenyl, —(C₂-C₃₀)alkynyl, —(C₁-C₃₀)alkyl-Rⁱ², —(C₁-C₃₀)alkyl-O—Rⁱⁱⁱ², —(C₁-C₃₀)alkyl-NR^{iv2}R^{v2}, —C(O)—R^{vi2}, and a radical selected from the group consisting of (XXII), (XXIII), (XXIV), (XXV), (XXVI), (XXVII), (XXVIII), (XXIX), (XXX), and (XXXI): 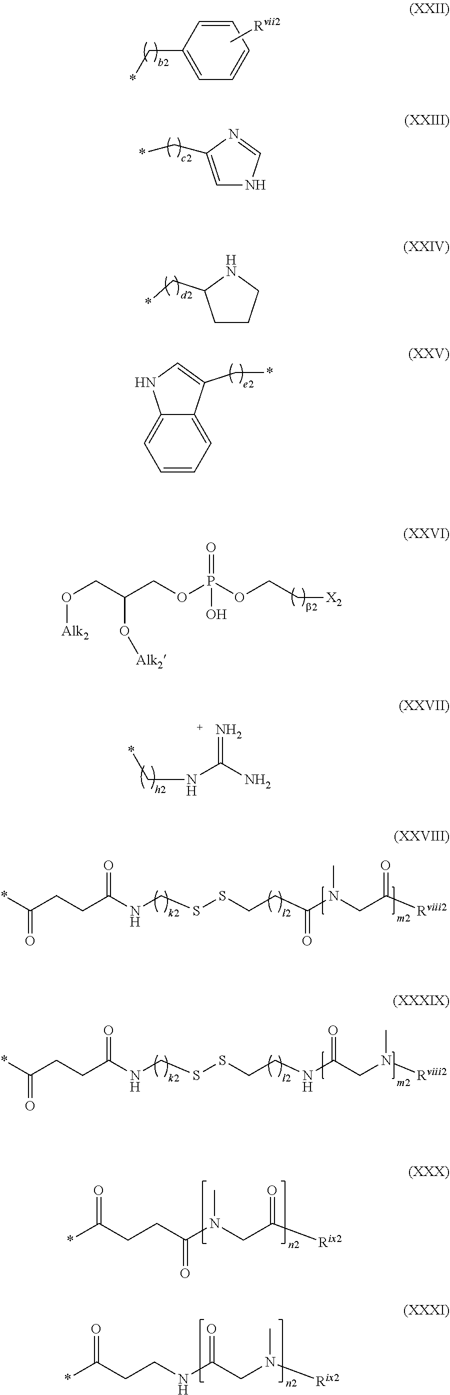
wherein “*” denotes the attaching point;
Rⁱ² is selected from the group consisting of H, —(C₁-C₃₀)alkyl, —(C₂-C₃₀)alkenyl, —(C₂-C₃₀)alkynyl, —O-alkyl(C₁-C₆), halogen, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NO₂, —CN, —OC(O)—(C₁-C₃₀)alkyl, —OC(O)O(C₁-C₃₀)alkyl, —OC(O)NH₂, —OC(O)N((C₁-C₃₀)alkyl)₂, —SH, —S(C₁-C₃₀)alkyl, —S(O)H, —S(O)(C₁-C₃₀)alkyl, and —SO₂(C₁-C₃₀)alkyl;
Rⁱⁱⁱ², R^{iv2}, and R^{v2} are independently selected from the group consisting of H, —(C₁-C₃₀)alkyl, —(C₂-C₃₀)alkenyl, —(C₁-C₃₀)alkylNH₂, —(C₁-C₃₀)alkyl-N((C₁-C₃₀)alkyl)₂, —(C₁-C₃₀)alkyl-NH(C₁-C₃₀)alkyl, and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI): 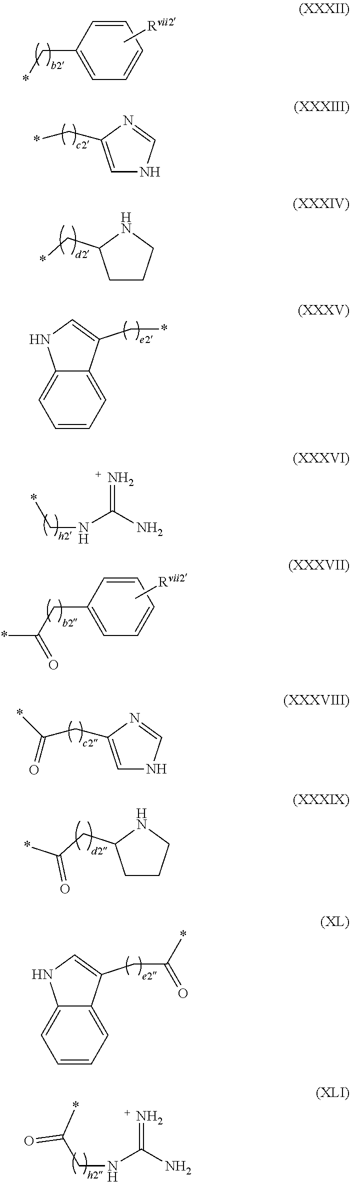 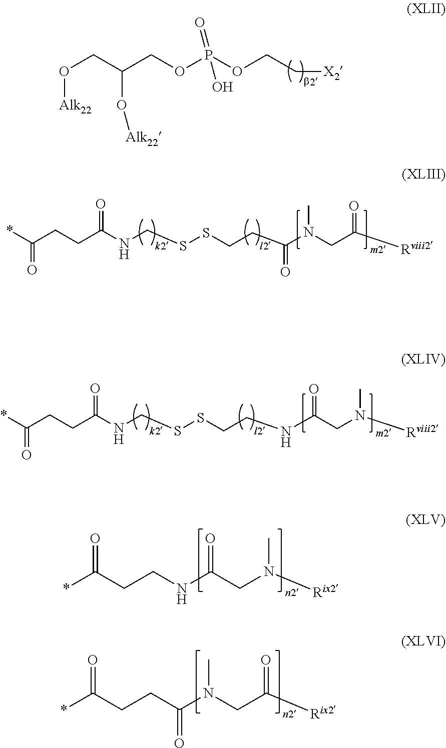
wherein R^{viii2}, R^{ix2}, R^{viii2′}, and R^{ix2″} are independently selected from the group consisting of H, —(C₁-C₁₂)alkyl, —(C₁-C₁₂)alkylNH₂, —(C₁-C₁₂)alkyl-N((C₁-C₁₂)alkyl)₂, —(C₁-C₁₂)alkyl-NH(C₁-C₁₂)alkyl, —O(C₁-C₁₂)alkyl, —COH, —CO(C₁-C₁₂)alkyl, and —O(C₂-C₃₀)alkenyl,
R^{vii2} and R^{vii2′} are independently selected from the group consisting of H, —(C₁-C₃₀)alkyl, —(C₂-C₃₀)alkenyl, —(C₂-C₃₀)alkynyl, —O-alkyl(C₁-C₁₂), F, Cl, Br, I, —CF₃, —OCF₃, —NO₂, —CN, —NH₂, —(C₁-C₃₀)alkylNH₂, —N((C₁-C₃₀)alkyl)₂, and —NH(C₁-C₃₀)alkyl,
R^{vi2} is selected from the group consisting of H, —OH, —(C₁-C₃₀)alkyl, —(C₂-C₃₀)alkenyl, —(C₁-C₃₀)alkyl-COOH, —(C₂-C₃₀)alkenyl-COOH, —(C₁-C₃₀)alkylNH₂, —NH₂, —(C₁-C₃₀)alkyl-N((C₁-C₃₀)alkyl)₂, —O—(C₁-C₃₀)alkyl, —NH(C₂-C₃₀)alkenyl, —(C₁-C₃₀)alkyl-NH(C₁-C₃₀)alkyl, —NH-oleic, —NH-noneic, —NH-lipoic, and —CH═CH(COOH)—CH₂—COOH;
wherein Alk₂, Alk₂₂, Alk₂′ and Alk₂₂′ are each independently selected from the group consisting of linear or branched —(C₁-C₃₀)alkyl and linear or branched —(C₂-C₃₀)alkenyl; β₂ and β₂′ are each independently an integer from 0 to 6, and X₂ and X₂′ are each independently selected from —NH—, —COO—, and —O—;
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of H, OH, halogen, —O(C₁-C₃₀)alkyl, —CF₃, —OCF₃, —NH₂, —(C₁-C₃₀)alkyl, —(C₂-C₃₀)alkenyl, —(C₂-C₃₀)alkynyl, —SH, —NHNH₂, —NHCH₃, —N(CH₃)₂, —NCH(CH₃)₂ and —(C₁-C₃₀)alkyl-OH;
b2, c2, d2, e2, h2, k2, l2, k2′, l2′, b2″, c2″, d2″, e2″, and h2″ are integers independently ranging from 1 to 20;
b2′, c2′, d2′, e2′, and h2′ are integers independently ranging from 0 to 20;
m2, n2, m2′, and n2′ are integers independently ranging from 1 to 200;
wherein R₃, R₄, R¹¹ and R¹³ are a biradical independently selected from the group consisting of —(C₁-C₆)alkyl-, —(C₁-C₆)alkyl-S—S—(C₁-C₆)alkyl-, —(C₁-C₆)alkyl-O—(C₁-C₆)alkyl-, and —(C₁-C₆)alkyl-NH—(C₁-C₆)alkyl-;
R₃, R₄, R₁₁ and R₁₃ are optionally substituted by one or more substituents selected from the group consisting of —NH₂ and —(C₁-C₆)alkyl-NH₂;
with the proviso that R₃ is absent when a=1, and R₁₁ is absent when a′=1;
R₅, R₈, R₁₀, R₁₂, R₁₆ and R₁₈ are a radical independently selected from the group consisting of H and —(C₁-C₆)alkyl;
wherein R₉ and R₁₀ are optionally combined together to form a proline ring moiety;
and wherein R₁₇ and R₁₈ are optionally combined together to form a proline ring moiety.

2. The compound of formula I according to claim 1, wherein α, α′ and α″ are 0.

3. The compound of formula I according to claim 1, wherein R₁ is a biradical selected from the group consisting of (III) and (IV): 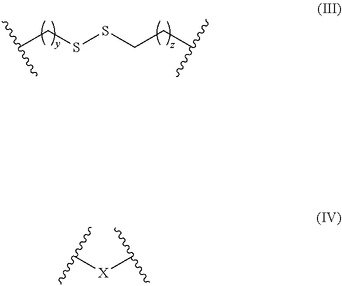
wherein the wavy lines denote the attaching points;
wherein y and z are integers independently ranging from 1 to 6;
X is a biradical selected from straight or branched —(C₁-C₁₂)alkylene-, —(C₁-C₆)alkyl-COO—, and a biradical selected from the group consisting of (V), (VI), (VII), (VIII), (IX), (X), (XI) and (XII);
wherein the straight or branched —(C₁-C₁₂)alkylene biradical of X is optionally substituted with one or more radicals selected from the group consisting of —OH, —NRₐR_{b}, —SH, —NHNH₂, —COOR_{c}, —CF₃, —OCF₃, and halogen;
Rₐ, R_{b} and R_{c} are radicals independently selected from the group consisting of H, phenyl, —(C₁-C₁₂)alkyl, —(C₂-C₃₀)alkenyl, —(C₁-C₁₂)alkylphenyl, and -phenyl(C₁-C₁₂)alkyl.

4. The compound of formula I according to claim 1, wherein
R₉ and R₁₇ are a radical independently selected from the group consisting of H, —(C₁-C₁₂)alkyl, —(C₂-C₃₀)alkenyl, —(C₁-C₁₂)alkyl-Rⁱ¹, —(C₁-C₁₂)alkyl-O—Rⁱⁱⁱ¹, —(C₁-C₁₂)alkyl-NR^{iv1}R^{v1}, —C(O)—R^{vi1}, —(C₁-C₁₂)alkyl-CO—NH₂ and a radical selected from the group consisting of (XIII), (XIV), (XV), (XVI), (XVII), (XVIII), (XIX), (XX), and (XXI);
Rⁱ¹ is selected from the group consisting of H, F, Cl, Br, I, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NH₂, —N((C₁-C₁₂)alkyl)₂, —NH(C₁-C₁₂)alkyl, —NHC(O)—(C₁-C₁₂)alkyl, —NHC(O)O(C₁-C₁₂)alkyl, —NHC(O)NH₂, —NHC(O)N(CH₃)₂, —NHS(O)₂(C₁-C₁₂)alkyl, —NHSO₂NH₂, —SH, —S(C₁-C₁₂)alkyl, —S(O)H, —S(O)(C₁-C₁₂)alkyl, —SO₂(C₁-C₁₂)alkyl, —SeH, —C(O)(C₁-C₁₂)alkyl, and —CON((C₁-C₁₂)alkyl)₂;
R^{vii1} is selected from the group consisting of H, methyl, ethyl, propyl, isopropyl, butyl, —(C₂-C₃₀)alkenyl, —(C₂-C₃₀)alkynyl, —O-alkyl(C₁-C₁₂), F, Cl, Br, I, —CF₃, —OCF₃, —NO₂, —CN, —NH₂, —(C₁-C₆)alkylNH₂, —N((C₁-C₆)alkyl)₂, and —NH(C₁-C₆)alkyl;
Rⁱⁱⁱ¹, R^{iv1} and R^{v1} are independently selected from the group consisting of H, —(C₁-C₁₂)alkyl, —(C₁-C₁₂)alkylNH₂, —(C₁-C₁₂)alkyl-N((C₁-C₁₂)alkyl)₂, and —(C₁-C₁₂)alkyl-NH(C₁-C₁₂)alkyl;
R^{vi1} is selected from the group consisting of H, —(C1-C₁₂)alkyl, —(C₂-C₃₀)alkenyl, —(C₁-C₁₂)alkylNH₂, —NH₂, —(C₁-C₁₂)alkyl-N((C₁-C₁₂)alkyl)₂, —NH(C₂-C₃₀)alkenyl, —(C₁-C₁₂)alkyl-NH(C₁-C₁₂)alkyl, —NH-oleic, —NH-noneic, and —NH-lipoic;
wherein Rⁱ¹, Rⁱⁱ¹, Rⁱⁱⁱ¹, R^{iv1}, R^{v1}, R^{vi1}, and R^{vii1} are optionally substituted with one or more substituents selected from the group consisting of —OH, F, Cl, Br, I, —O(C₁-C₆)alkyl, —CF₃, —OCF₃, —NH₂, —(C₁-C₆)alkyl, —SH, —NHNH₂, —NHCH₃, —N(CH₃)₂, —NCH(CH₃)₂ and —(C₁-C₆)alkyl-OH;
b1, c1, d1, e1, f1, g1, h1, i1, and j1 are integers independently ranging from 1 to 6;
wherein R₉ and R₁₀ are optionally combined together to form a proline ring moiety;
and wherein R₁₇ and R₁₈ are optionally combined together to form a proline ring moiety.

5. The compound of formula I according to claim 1, wherein
R₉ and R₁₇ are a radical independently selected from H, methyl, ethyl, propyl, isopropyl, butyl, n-butyl, —(C₂-C₃₀)alkenyl, —CH₂SCH₃, —CH₂CH₂SCH₃, —CH₂CH₂SCH₂CH₃, —CH₂SCH₂CH₃, —CH₂SH, —CH₂—SeH, —CH₂CH₂SH, —CH₂NH₂, —CH₂CH₂NH₂, —CH₂CH₂CH₂NH₂, —CH₂CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), —C(O)H, —C(O)OCH₃, —C(O)OCH₂CH₃, —C(O)OCH(CH₃)₂, —C(O)CH₂NH₂, —C(O)CH₂CH₂NH₂, —C(O)CH(CH₃)CH₂NH₂, —C(O)CH₂NHCH₃, —C(O)CH₂NHCH₂CH₃, —C(O)CH₂CH₂NHCH₂CH₃, —C(O)CH₂CH₂CH₂NHCH₂CH₃, —C(O)CH₂CH₂CH₂NHCH₂CH₂CH₃, —C(O)CH₂CH(CH₃)CH₂NHCH₃, —C(O)CH₂CH(CH₃)CH₂NHCH₂CH₃, —C(O)CH₂CH₂NHCH₂CH₂NH₂, —C(O)CH₂CH₂CH₂NHCH₂CH₂CH₂NH₂, —C(O)CH₂CH₂N((CH(CH₃)₂)), —C(O)CH₂CH₂CH₂N((CH(CH₃)₂)), —C(O)CH₂CH₂NH(CH(CH₃)₂), —C(O)CH₂CH₂CH₂NH(CH(CH₃)₂), —CH₂COOCH₃, —CH₂CH₂COOCH₃, —CH₂COOCH₂CH₃, —CH₂CH₂COOCH₂CH₃, —CH₂COOCH(CH₃)₂, —CH₂CONH₂, —CH₂CH₂CONH₂, —CH₂CH₂CH₂CONH₂, —CONH-oleic, —CONH-noneic, —CONH-lipoic, and a radical selected from the group consisting of (XIII), (XIV), (XV), (XVI), (XVII), (XVIII), (XIX), (XX), and (XXI);
wherein R^{vii1} is selected from the group consisting of H, methyl, ethyl, propyl, isopropyl, butyl, —(C₂-C₃₀)alkenyl, —(C₂-C₃₀)alkynyl, —O-alkyl(C₁-C₁₂), F, Cl, Br, I, —CF₃, —OCF₃, —NO₂, —CN, —NH₂, —(C₁-C₆)alkylNH₂, —N((C₁-C₆)alkyl)₂, and —NH(C₁-C₆)alkyl;
and wherein b1, c1, d1, e1, f1, g1, h1, i1, and j1 are integers independently ranging from 1 to 6;
wherein R₉ and R₁₀ are optionally combined together to form a proline ring moiety;
and wherein R₁₇ and R₁₈ are optionally combined together to form a proline ring moiety.

6. The compound of formula I according to claim 1, wherein
R₉ and R₁₇ are a radical independently selected from the group consisting of H, methyl, ethyl, propyl, isopropyl, butyl, n-butyl, —CH₂SCH₃, —CH₂CH₂SCH₃, —CH₂CH₂SCH₂CH₃, —CH₂SCH₂CH₃, —CH₂SH, —CH₂CH₂SH, —CH₂SeH, —CH₂NH₂, —CH₂CH₂NH₂, —CH₂CH₂CH₂NH₂, —CH₂CH₂CH₂CH₂NH₂, and a radical selected from the group consisting of (XIII), (XIV), (XV), (XVI), (XVII), (XVIII), (XIX), (XX), and (XXI);
wherein R^{vii1} is selected from H, methyl, ethyl, propyl, isopropyl, butyl, —(C₂-C₃₀)alkenyl, —(C₂-C₃₀)alkynyl, —OAlkyl(C₁-C₆), F, Cl, Br, I, —CF₃, —OCF₃, —NO₂, —CN, —NH₂, —CH₂NH₂, —CH₂CH₂NH₂, —CH(CH₃)NH₂, —CH₂CH(CH₃)NH₂, —CH₂CH₂CH₂NH₂, —N(CH₃)₂, —N(CH₂CH₃)₂, —NCH(CH₃)₂, —NHCH₃, —NHCH₂CH₃, and —NHCH(CH₃)₂;
and wherein b1, c1, d1, e1, f1, g1, h1, i1, and j1 are integers independently ranging from 1 to 4;
wherein R₉ and R₁₀ are optionally combined together to form a proline ring moiety;
and wherein R₁₇ and R₁₈ are optionally combined together to form a proline ring moiety.

7. The compound of formula I according to claim 1, wherein
R₆, R₇, R₁₄, R₁₅ and R₁₉ are independently selected from the group consisting of H, —(C₁-C₃₀)alkyl, —(C₂-C₃₀)alkenyl, —(C₂-C₃₀)alkynyl, —(C₁-C₃₀)alkyl-Rⁱ², —(C₁-C₃₀)alkyl-O—Rⁱⁱⁱ², —(C₁-C₃₀)alkyl-NR^{iv2}R^{v2}, —C(O)—R^{vi2}, and a radical selected from the group consisting of (XXII), (XXIII), (XXIV), (XXV), (XXVI), (XXVII), (XXVIII), (XXIX), (XXX), and (XXXI);
Rⁱ² is selected from the group consisting of H, —(C₁-C₁₂)alkyl, —O-alkyl(C₁-C₆), F, Cl, Br, I, —CF₃, —OCF₃, isoxazole, oxazole, furan, oxolane, thiole, thiophene, N-methylpyrrole, pyrrole, pyrrolidine, pyrane, pyridine, piperidine, thiazole, dioxane, morpholine, pyrimidine, —NO₂, —CN, —OC(O)—(C₁-C₁₂)alkyl, —OC(O)O(C₁-C₁₂)alkyl, —OC(O)NH₂, —OC(O)N((C₁-C₁₂)alkyl)₂, —SH, —S(C₁-C₁₂)alkyl, —S(O)H, —S(O)(C₁-C₁₂)alkyl, —SO₂(C₁-C₁₂)alkyl;
Rⁱⁱⁱ², R^{iv2}, and R^{v2} are independently selected from the group consisting of H, —(C₁-C₁₂)alkyl, —(C₂-C₃₀)alkenyl, —(C₁-C₃₀)alkylNH₂, —(C₁-C₁₂)alkyl-N((C₁-C₁₂)alkyl)₂, —(C₁-C₁₂)alkyl-NH(C₁-C₁₂)alkyl, and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI);
wherein R^{viii2}, R^{ix2}, R^{viii2′}, and R^{ix2″} are independently selected from the group consisting of H, —(C₁-C₆)alkyl, —(C₁-C₆)alkylNH₂, —(C₁-C₆)alkyl-N((C₁-C₆)alkyl)₂, —(C₁-C₆)alkyl-NH(C₁-C₆)alkyl, —O(C₁-C₆)alkyl, —COH, —CO(C₁-C₆)alkyl, and —O(C₂-C₁₂)alkenyl,
R^{vii2} and R^{vii2′} are independently selected from the group consisting of H, —(C₁-C₁₂)alkyl, —(C₂-C₃₀)alkenyl, —(C₂-C₃₀)alkynyl, —O(C₁-C₆)Alkyl, F, Cl, Br, I, —CF₃, —OCF₃, —NO₂, —CN, —NH₂, —(C₁-C₁₂)alkylNH₂, —N((C₁-C₁₂)alkyl)₂, and —NH(C₁-C₁₂)alkyl;
R^{vi2} is selected from the group consisting of H, —OH, —(C₁-C₁)alkyl, —(C₂-C₃₀)alkenyl, —(C₁-C₁₂)alkyl-COOH, —(C₂-C₃₀)alkenyl-COOH, —(C₁-C₁₂)alkylNH₂, —(C₁-C₁₂)alkyl-N((C₁-C₁₂)alkyl)₂, —O—(C₁-C₁₂)alkyl, —NH(C₂-C₁₂)alkenyl, —(C₁-C₁₂)alkyl-NH(C₁-C₁₂)alkyl, —NH-oleic, —NH-noneic, —NH-lipoic, and —CH═CH(COOH)—CH₂—COOH,
wherein Alk₂, Alk₂₂, Alk₂′ and Alk₂₂′ are each independently selected from the group consisting of linear or branched —(C₁-C₁₂)alkyl and linear or branched —(C₂-C₃₀)alkenyl; β₂ and β₂′ are each independently an integer from 0 to 6, and X₂ and X₂′ are each independently selected from the group consisting of —NH—, —COO—, and —O—;
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of OH, F, Cl, Br, I, —O(C₁-C₆)alkyl, —CF₃, —OCF₃, —NH₂, —(C₁-C₆)alkyl-NHNH₂, —NHCH₃, —N(CH₃)₂, —NCH(CH₃)₂, and —(C₁-C₆)alkyl-OH;
b2, c2, d2, e2, h2, k2, l2, k2′, l2′, b2″, c2″, d2″, e2″, and h2″ are integers independently ranging from 1 to 6;
b2′, c2′, d2′, e2′, and h2′ are integers independently ranging from 0 to 6; and
m2, n2, m2′, and n2′ are integers independently ranging from 1 to 150.

8. The compound of formula I according to claim 1, wherein
R₆, R₇, R₁₄, R₁₅, and R₁₉ are independently selected from the group consisting of H, methyl, ethyl, propyl, isopropyl, butyl, —CH₂OCH₃, —CH₂CH₂OCH₃, —CH₂CH₂OCH₂CH₃, —CH₂CH₂OCH(CH₃)₂, —CH₂OCH₂CH₃, —CH₂OCH(CH₃)₂, —(C₂-C₃₀)alkenyl, —(C₂-C₃₀)alkynyl, —CH₂SCH₃, —CH₂CH₂SCH₃, —CH₂CH₂SCH₂CH₃, —CH₂SCH₂CH₃, —CH₂NH₂, —CH₂CH₂NH₂, —CH₂CH₂CH₂NH₂, —CH₂CH(CH₃) CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), —C(O)H, —C(O)OCH₃, —C(O)OCH₂CH₃, —C(O)OCH(CH₃)₂, —C(O)CH₂NH₂, —C(O)CH₂CH₂NH₂, —C(O)CH(CH₃)CH₂NH₂, —C(O)CH₂NHCH₃, —C(O)CH₂NHCH₂CH₃, —C(O)CH₂CH₂NHCH₂CH₃, —C(O)CH₂CH₂CH₂NHCH₂CH₃, —C(O)CH₂CH₂CH₂NHCH₂CH₂CH₃, —C(O)CH₂CH(CH₃)CH₂NHCH₃, —C(O)CH₂CH(CH₃)CH₂NHCH₂CH₃, —C(O)CH₂CH₂NHCH₂CH₂NH₂, —C(O)CH₂CH₂CH₂NHCH₂CH₂CH₂NH₂, —C(O)CH₂CH₂N((CH(CH₃)₂)), —C(O)CH₂CH₂CH₂N((CH(CH₃)₂)), —C(O)CH₂CH₂NH(CH(CH₃)₂), —C(O)CH₂CH₂CH₂NH(CH(CH₃)₂), —CH₂COOH, —CH₂CH₂COOH, —CH₂COOCH₃, —CH₂CH₂COOCH₃, —CH₂COOCH₂CH₃, —CH₂CH₂COOCH₂CH₃, —CH₂COOCH(CH₃)₂, —(C₁-C₆)alkyl-Rⁱ², —(C₁-C₆)alkyl-O—Rⁱⁱⁱ², —(C₁-C₆)alkyl-NR^{v2}R^{v2}, —CONH-oleic, —CONH-noneic, —CONH-lipoic, and a radical selected from the group consisting of (XXII), (XXIII), (XXIV), (XXV), (XXVI), (XXVII), (XXVIII), (XXIX), (XXX), and (XXXI);
Rⁱ² is selected from the group consisting of imidazole, N-methylpyrrole, pyrrole, pyrrolidine, pyrane, pyridine, piperidine, pyrimidine, —OC(O)NH₂, and —OC(O)N((C₁-C₆)alkyl)₂;
Rⁱⁱⁱ², R^{iv2}, and R^{v2} are independently selected from the group consisting of —(C₂-C₃₀)alkenyl, —(C₂-C₃₀)alkynyl, —(C₁-C₆)alkyl-NH₂, —(C₁-C₆)alkyl-N((C₁-C₆)alkyl)₂, —(C₁-C₆)alkyl-NH(C₁-C₆)alkyl, and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI),
R^{vii2} and R^{vii2′} are independently selected from H, methyl, ethyl, propyl, isopropyl, butyl, —OAlkyl(C₁-C₆), F, Cl, Br, I, —CF₃, —OCF₃, —NO₂, —CN, —NH₂, —(C₁-C₆)alkyl-NH₂, —N((C₁-C₆)alkyl)₂, —NH(C₁-C₆)alkyl;
wherein R^{viii2}, R^{ix2}, R^{vii2′}, and R^{ix2″} are independently selected from H, —(C₁-C₆)alkyl, —(C₂-C₆)alkenyl, —(C₂-C₆)alkynyl, —(C₁-C₆)alkylNH₂, —(C₁-C₆)alkyl-N((C₁-C₆)alkyl)₂, —(C₁-C₆)alkyl-NH(C₁-C₆)alkyl;
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of OH, F, Cl, Br, —OCH₃, —OCH₂CH₃, —OCH(CH₃)₂, —CF₃, —OCF₃, —NH₂, —CH₃, —CH₂CH₃, —CH(CH₃)₂, —CH₂OH, —CH₂CH₂OH, and —CH₂CH(OH)CH₃;
b2, c2, d2, e2, h2, k2, l2, k2′, l2′, b2″, c2″, d2″, e2″, and h2″ are integers independently ranging from 1 to 4;
b2′, c2′, d2′, e2′, and h2′ are integers independently ranging from 0 to 4; and
m2, n2, m2′, and n2′ are integers independently ranging from 1 to 120.

9. The compound of formula I according to claim 1, wherein
R₁ is a biradical selected from the group consisting of —CH₂CH₂—S—S—CH₂CH₂—, —CH₂CH₂CH₂—S—S—CH₂CH₂CH₂—, —CH₂—, —CH₂CH₂—, —CH₂CH(CH₃)CH₂—, —CH₂CH(CH₂CH₃)CH₂—, —CH₂CH₂CH₂CH₂—, —CH₂COO—, —CH₂CH₂COO—, —CH₂CHCH₃COO—, —CH₂CH₂CH₃CH₂COO—, and a biradical selected from the group consisting of (V), (VI), (VII), (VIII), (IX), (X), (XI), and (XII);
R₉ and R₁₇ are a radical independently selected from the group consisting of H, methyl, ethyl, propyl, isopropyl, butyl, n-butyl, —CH₂SCH₃, —CH₂CH₂SCH₃, —CH₂CH₂SCH₂CH₃, —CH₂SCH₂CH₃, —CH₂SH, —CH₂CH₂SH, —CH₂SeH, —CH₂NH₂, —CH₂CH₂NH₂, —CH₂CH₂CH₂NH₂, —CH₂CH₂CH₂CH₂NH₂, and a radical selected from the group consisting of (XIII), (XIV), (XV), (XVI), (XVII), (XVIII), (XIX), (XX), and (XXI);
wherein R^{vii1} is selected from H, methyl, ethyl, propyl, isopropyl, butyl, —(C₂-C₃₀)alkenyl, —(C₂-C₃₀)alkynyl, —OCH₃, —OCH₂CH₃, F, Cl, Br, I, —CF₃, —OCF₃, —NO₂, —CN, —NH₂, —CH₂NH₂, —CH₂CH₂NH₂, —CH(CH₃)NH₂, —CH₂CH(CH₃)NH₂, —CH₂CH₂CH₂NH₂, —N(CH₃)₂, —N(CH₂CH₃)₂, —NCH(CH₃)₂, —NHCH₃, —NHCH₂CH₃, and —NHCH(CH₃)₂;
wherein b1, c1, d1, e1, f1, g1, h1, i1, and j1 are integers independently ranging from 1 to 6;
wherein R₉ and R₁₀ are optionally combined together to form a proline ring moiety;
and wherein R₁₇ and R₁₈ are optionally combined together to form a proline ring moiety;
wherein r, s, t and u are integers independently ranging from 0 to 250, and wherein at least one of r or t is ≥1.

10. The compound of formula I according to claim 1, wherein
R₁ is a biradical selected from the group consisting of —CH₂CH₂—S—S—CH₂CH₂—, —CH₂CH₂CH₂—S—S—CH₂CH₂CH₂—, —CH₂—, —CH₂CH₂—, —CH₂CH(CH₃)CH₂—, —CH₂CH(CH₂CH₃)CH₂—, —CH₂CH₂CH₂CH₂—, —CH₂COO—, —CH₂CH₂COO—, —CH₂CHCH₃COO—, —CH₂CH₂CH₃CH₂COO—, and a biradical selected from the group consisting of (V), (VI), (VII), (VIII), (IX), (X), (XI) and (XII);
R₉ and R₁₇ are a radical independently selected from H, methyl, ethyl, propyl, isopropyl, butyl, n-butyl, —CH₂SCH₃, —CH₂CH₂SCH₃, —CH₂CH₂SCH₂CH₃, —CH₂SCH₂CH₃, —CH₂SH, —CH₂CH₂SH, —CH₂SeH, —CH₂NH₂, —CH₂CH₂NH₂, —CH₂CH₂CH₂NH₂, —CH₂CH₂CH₂CH₂NH₂, and a radical selected from the group consisting of (XIII), (XIV), (XV), (XVI), (XVII), (XVIII), (XIX), (XX), and (XXI);
R^{vii1} is selected from H, methyl, ethyl, propyl, isopropyl, butyl, —OCH₃, —OCH₂CH₃, F, Cl, Br, I, —CF₃, —OCF₃, —NO₂, —CN, —NH₂, —CH₂NH₂, —CH₂CH₂NH₂, —CH₂CH(CH₃)NH₂, —CH₂CH₂CH₂NH₂, —N(CH₃)₂, —N(CH₂CH₃)₂, —NCH(CH₃)₂, —NHCH₃, —NHCH₂CH₃, and —NHCH(CH₃)₂;
b1, c1, d1, e1, f1, g1, h1, i1, and j1 are integers independently ranging from 1 to 6;
wherein R₉ and R₁₀ are optionally combined together to form a proline ring moiety;
and wherein R₁₇ and R₁₈ are optionally combined together to form a proline ring moiety;
R₆, R₇, R₁₄, R₁₅ and R₁₉ are independently selected from H, methyl, ethyl, propyl, isopropyl, butyl, —CH₂OCH₃, —CH₂CH₂OCH₃, —CH₂CH₂OCH₂CH₃, —CH₂CH₂OCH(CH₃)₂, —CH₂OCH₂CH₃, —CH₂OCH(CH₃)₂, —(C₂-C₃₀)alkenyl, —(C₂-C₃₀)alkynyl, —CH₂SCH₃, —CH₂CH₂SCH₃, —CH₂SH, —CH₂SeH, —CH₂CH₂SH, —CH₂CH₂SCH₂CH₃, —CH₂SCH₂CH₃, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), —C(O)H, —C(O)OCH₃, —C(O)OCH₂CH₃, —C(O)OCH(CH₃)₂, —C(O)CH₂NH₂, —C(O)CH₂CH₂NH₂, —C(O)CH(CH₃)CH₂NH₂, —C(O)CH₂NHCH₃, —C(O)CH₂NHCH₂CH₃, —C(O)CH₂CH₂NHCH₂CH₃, —C(O)CH₂CH₂CH₂NHCH₂CH₃, —C(O)CH₂CH₂CH₂NHCH₂CH₂CH₃, —C(O)CH₂CH(CH₃)CH₂NHCH₃, —C(O)CH₂CH(CH₃)CH₂NHCH₂CH₃, —C(O)CH₂CH₂NHCH₂CH₂NH₂, —C(O)CH₂CH₂CH₂NHCH₂CH₂CH₂NH₂, —C(O)CH₂CH₂N((CH(CH₃)₂)), —C(O)CH₂CH₂CH₂N((CH(CH₃)₂)), —C(O)CH₂CH₂NH(CH(CH₃)₂), —C(O)CH₂CH₂CH₂NH(CH(CH₃)₂), —CH₂COOH, —CH₂CH₂COOH, —CH₂COOCH₃, —CH₂CH₂COOCH₃, —CH₂COOCH₂CH₃, —CH₂CH₂COOCH₂CH₃, —CH₂COOCH(CH₃)₂, —(C₁-C₆)alkyl-Rⁱ², —(C₁-C₆)alkyl-O—Rⁱⁱⁱ², —(C₁-C₆)alkyl-NR^{iv2}R^{v2}, —CONH-oleic, —CONH-noneic, —CONH-lipoic, and a radical selected from the group consisting of (XXII), (XXIII), (XXIV), (XXV), (XXVI), (XXVII), (XXVIII), (XXIX), (XXX), and (XXXI);
Rⁱ² is selected from the group consisting of imidazole, N-methylpyrrole, pyrrole, pyrrolidine, pyrane, pyridine, piperidine, pyrimidine, —OC(O)NH₂, and —OC(O)N((C₁-C₆)alkyl)₂;
Rⁱⁱⁱ², R^{iv2}, and R^{v2} are independently selected from H, —(C₂-C₃₀)alkenyl, —(C₂-C₃₀)alkynyl, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), —CH₂CH₂CH₂NH(CH(CH₃)₂), and a radical selected from the group consisting of (XXXII), (XXXIII), (XXXIV), (XXXV), (XXXVI), (XXXVII), (XXXVIII), (XXXIX), (XL), (XLI), (XLII), (XLIII), (XLIV), (XLV), and (XLVI);
wherein R^{vii2} and R^{vii2′} are independently selected from H, methyl, ethyl, propyl, isopropyl, butyl, F, Cl, Br, I, —CF₃, —OCF₃, —NO₂, —CN, —NH₂, and —(C₁-C₆)alkyl-NH₂;
wherein R^{viii2}, R^{ix2}, R^{viii2′}, and R^{ix2″} are independently selected from H, methyl, ethyl, propyl, isopropyl, —CH₂NH₂, —CH₂CH₂NH₂, —CH₂CH₂CH₂NH₂, —CH₂CH(CH₃)CH₂NH₂, —CH₂NHCH₃, —CH₂NHCH₂CH₃, —CH₂CH₂NHCH₃, —CH₂CH₂NHCH₂CH₃, —CH₂CH₂CH₂NHCH₃, —CH₂CH₂CH₂NHCH₂CH₃, —CH₂CH(CH₃)CH₂NHCH₃, —CH₂CH(CH₃)CH₂NHCH₂CH₃, —CH₂CH₂NHCH₂CH₂NH₂, —CH₂CH₂CH₂NHCH₂CH₂CH₂NH₂, —CH₂CH₂N((CH(CH₃)₂)), —CH₂CH₂CH₂N((CH(CH₃)₂)), —CH₂CH₂NH(CH(CH₃)₂), and —CH₂CH₂CH₂NH(CH(CH₃)₂);
wherein Rⁱ², Rⁱⁱⁱ², R^{iv2}, R^{v2}, R^{vi2}, R^{vii2}, R^{viii2}, R^{ix2}, R^{viii2′}, and R^{ix2′} are optionally substituted by one or more substituents selected from the group consisting of OH, F, Cl, Br, —OCH₃, —OCH₂CH₃, —OCH(CH₃)₂, —CF₃, —OCF₃, —NH₂, —CH₃, —CH₂CH₃, —CH(CH₃)₂, —CH₂OH, —CH₂CH₂OH, and —CH₂CH(OH)CH₃;
b2, c2, d2, e2, h2, k2, l2, k2′, l2′, b2″, c2″, d2″, e2″, and h2″ are integers independently ranging from 1 to 4;
b2′, c2′, d2′, e2′, and h2′ are integers independently ranging from 0 to 4; and
m2, n2, m2′, and n2′ are integers independently ranging from 1 to 120.

11. The compound of formula I according to claim 1, which is selected from

| | |
|---|---|
| CP5 | St-PAspDET(100) |
| CP6 | St-PAspDET(50) |
| CP7 | St-S-S-PAspDET(35) |
| CP8 | St-S-S-PAspDET(50) |
| CP9 | St-S-S-PAspDET(100) |
| CP10 | St-PAspDET(71)/DIIPA(29) |
| CP11 | St-S-S-PAspDET(45)/DIIPA(21)) |
| CP12 | St-S-S-PAspDET(78)/DIIPA(21) |
| CP13 | St-S-S-PAspDET(91)/DIIPA(29) |
| CP14 | St-S-S-PAspDET(73)/DIIPA(34) |
| CP17 | St-S-S-PAspDET(47)-co-PLeu(9) |
| CP18 | St-S-S-PAspDET(54)-co-PPhe(4) |
| CP19 | St-S-S-PAspDET(57)-co-PPhe(5) |
| CP20 | St-S-S-PAspDET(37)-co-PPhe(9) |
| CP21 | St-S-S-PAspDET(48)/DIIPA(18)-co-PLeu(4) |
| CP22 | St-S-S-PAspDET(31)/DIIPA(12)-co-PLeu(7) |
| CP23 | St-S-S-PAspDET(41)/DIIPA(10)-co-PPhe(5) |
| CP30 | St-S-S-PAspDET(42)-b-Psar(37) |
| CP31 | St-S-S-PAspDET(41)-b-Psar(22) |
| CP32 | St-S-S-PAspDET(46)-co-PLeu(8)-b-PSar(50) |
| CP33 | St-S-S-PAspDET51-co-PPhe5-b-PSar44 |
| CP34 | St-S-S-PAspDET48-co-PPhe9-b-PSar36 |
| CP35 | St-S-S-PAspDET(51)/DIIPA(20)-b-PSar(58) |
| CP36 | St-S-S-PAspDET(38)/DIIPA(21)-co-PLeu(11)-b-PSar(50) |
| CP37 | St-S-S-PAspDET(46)/DIIPA(13)-co-PPhe(11)-b-PSar(28) |
| CP38 | St-S-S-PAspDET(100)-graft into-[Succ-PSar(47)- |
| | Galactosamine]3 |
| CP39 | St-S-S-PAspDET(91)/DIIPA(29)-graft into-[Succ-PSar(23)- |
| | iPr]6 |
| CP40 | St-S-S-PAspDET(100)-graft into-[Succ-ethyl-S-S-ethyl- |
| | PSar(28)]3-COCH₃ |
| CP41 | St-S-S-PAspDET(100)-graft into-[Succ-ethyl-S-S-ethyl- |
| | PSar(28)]2-COCH₃ |
| CP42 | St-S-S-PAspDET(100)-graft into-[Succ-ethyl-S-S-ethyl- |
| | PSar(28)]1-COCH₃ |
| CP43 | St-S-S-PAspDET(100)-graft into-[Succ-ethyl-S-S-ethyl- |
| | PSar(28)]6-COCH₃ |
| CP44 | St-S-S-PAspDET(57)-co-PPhe(5)-graft into-[Succ-ethyl- |
| | S-S-ethyl-PSar(28)]2-COCH₃ |
| CP45 | St-S-S-PAspTEP(100) |
| CP46 | St-S-S-PAspImidazolamine(100) |
| CP48 | St-S-S-PAspDET(49)/Imidazolamine(17) |
| CP49 | St-S-S-PAspDET(86)/Imidazolamine(31) |
| CP50 | St-AlaVal-PAspDET(87)/PPhe(10) |
| CP51 | St-S-S-PAspDET(36)/Imidazolamine(12)-co-PPhe(5) |
| CP52 | St-S-S-PAspDET(72)-graft into-lipoic acid(5) |
| CP53 | St-S-S-PAspDET(65)/Imidazolamine(26) graft into-oleic acid(5) |
| CP54 | St-S-S-PAspDET(65)/Imidazolamine(26) graft into-oleic |
| | acid(5)/nonanoic acid(13) |
| CP55 | St-[Lys(3)]PAspDET(100) |
| CP58 | St-S-S-PAspDET(100)-b-PPro(10) |
| CP59 | St-S-S-PAspDET(119)-b-PPro(14) |

wherein the numerical values mentioned in parenthesis refer to the polymerization degree for each monomeric unit and wherein each polymerization degree value is subject to a reasonable uncertainty which is within the DP range ±20%.

12. A conjugate comprising a radical derived from a compound of formula (I) according to claim 1, which is covalently attached to at least one labeling or imaging agent, or to a cell-targeting agent.

13. A polymer complex comprising the compound of formula (I) according to claim 1, and one or more active agents selected from the group consisting of pharmaceutically active agent, veterinary active agent, cosmetically active agent, diagnostically active agents, nucleic acids, peptides, antibodies, aptamers, proteins, and mixtures thereof.

14. The polymer complex according to claim 13, wherein the at least one active agent is selected from the group consisting of low molecular weight drugs, peptides, proteins, antibodies, nucleic acids, aptamers, and combinations thereof.

15. The polymer complex according to claim 14, wherein the nucleic acid is selected from the group consisting of a DNA/RNA hybrid, a short interfering RNA (siRNA), a microRNA (miRNA), sgRNA, a donor DNA, a self-amplifying/replicating RNA, a circular RNA (oRNA), a plasmid DNA (pDNA), a closed-linear DNA (clDNA), a short hairpin RNA (shRNA), messenger RNA (mRNA), and antisense RNA (aRNA), a messenger RNA (mRNA), a CRISPR guide RNA, an antisense nucleic acid, a decoy nucleic acid, an aptamer, and a ribozyme.

16. The polymer complex according to claim 15, wherein the nucleic acid is clDNA.

17. A composition comprising at least one conjugate according to claim 12 together with one or more pharmaceutically, diagnostically, veterinary or cosmetically acceptable excipients or carriers.

18. A method of using a therapeutic product for medicine, wherein the therapeutic product is a conjugate as defined in claim 12.

19. A method of delivering a nucleic acid into a cell, wherein the method comprises using a device comprising the polymer complex according to claim 15.

20. A method of delivering a nucleic acid into a target cell, which comprises contacting a solution that contains the polymer complex according to claim 15 to an animal with the target cell, so that the complex can be introduced into the target cell; transferring the complex from an endosome of the target cell to cytoplasm of the target cell; dissociating the complex in the target cell; and releasing the nucleic acid into the cytoplasm.

21. A method of transfecting a cell comprising contacting the cell with the polymer complex according to claim 13.
